(19) United States
(12) Patent Application Publication
(10) Pub. No.: US 2021/0002373 A1
(43) Pub. Date: Jan. 7, 2021

(54) KLRG1 Binding Compositions and Methods of Use Thereof

(71) Applicant: NextCure, Inc., Beltsville, MD (US)

(72) Inventors: Linda LIU, Clarksville, MD (US); Dallas Benjamin Flies, Rockville, MD (US)

(21) Appl. No.: 16/976,630

(22) PCT Filed: Mar. 1, 2019
(86) PCT No.: PCT/US2019/020220

§ 371 Date: Aug. 28, 2020

Related U.S. Application Data
(60) Provisional application No. 62/637,034, filed on Mar. 1, 2018.

(51) Int. Cl. C07K 16/28

(52) U.S. Cl. CPC C07K 16/2851; C07K 2317/71; C07K 2317/24; C07K 2317/52

(57) ABSTRACT

Compositions that bind to killer cell lectin-like receptor G1 (KLRG1) or its ligands are provided. The compositions can agonize or antagonize signal transduction through KLRG1. For example, some compositions either inhibit or block signal transduction through KLRG1 while other compositions induce or enhance signal transduction through KLRG1. In some embodiments, the compositions are anti-KLRG1 antibodies, KLRG1 fusion proteins, or KLRG1 ligand fusion proteins. One embodiment provides antibodies and antigen binding fragments thereof that immuno specifically bind KLRG1 and block, inhibit or reduce signal transduction through KLRG1. In another embodiment, the antibodies specifically bind to human KLRG1 under physiological conditions. In certain embodiments, the antibodies bind to KLRG1 that is arrayed on the surface of a cell; arrayed on the surface of a cell at an endogenous concentration; arrayed on the surface of a live cell, and inhibits or blocks interaction of KLRG1 and a ligand of KLRG1; or a combination thereof. In a preferred embodiment, the antibody or antigen binding fragment thereof binds to the extracellular domain of KLRG1 expressed on an immune cell including, but not limited to natural killer cells and T cells. Methods of using the compositions are also provided.

### CROSS-REFERENCE TO RELATED APPLICATIONS

This application claims benefit of and priority to U.S. Provisional Patent Application Nos. 62/637,034 filed on Mar. 1, 2018, which is incorporated by reference in its entirety.

### REFERENCE TO A SEQUENCE LISTING

The Sequence Listing submitted on Mar. 1, 2019, as a text file named “064467_004PCT_txt” created on Feb. 27, 2019, and having a size of 121,955 bytes is hereby incorporated by reference pursuant to 37 C.F.R. § 1.52(e)(5).

### TECHNICAL FIELD OF THE INVENTION

The invention is generally directed to compositions that bind to KLRG1 and methods of use thereof. Representative KLRG1 binding compositions include, but are not limited to antibodies and fusion proteins.

### BACKGROUND OF THE INVENTION

For 2016 the National Cancer Institute estimated that 1,685,210 new cases of cancer would be diagnosed in the United States and 595,690 people would die from the disease (www.cancer.gov). National expenditures for cancer care in the United States totaled nearly $125 billion in 2010 and could reach $156 billion in 2020. Clearly cancer has an enormous impact both on health and on the economy.

Because the human immune system is a complex system of cells and antibodies that involves signal transduction across multiple cell surface receptors, there is no one drug that can be used to successfully to treat all cancers. The use of monoclonal antibodies (mAbs) for cancer therapy has achieved considerable success in recent years (Scott, A., et al., Nature Reviews Cancer, 12:278-287 (2012)). More recently, mAbs to checkpoint inhibitors have been significant success in treating cancer.

PD-1 is a checkpoint protein on immune cells called T cells. It normally acts as a type of “off switch” that helps keep the T cells from attacking other cells in the body. It does this when it attaches to PD-L1, a protein on some normal (and cancer) cells. When PD-1 binds to PD-L1, it basically tells the T cell to leave the other cell alone. Some cancer cells have large amounts of PD-L1, which helps them evade immune attack.

Monoclonal antibodies that target either PD-1 or PD-L1 can block this binding and boost the immune response against cancer cells. These drugs have shown a great deal of promise in treating certain cancers. Examples of mAbs that target PD-1 include: Pembrolizumab and Nivolumab. Although these drugs have had very good results, they are not approved for all types of cancer. Additionally, these drugs were associated with the occurrence of side effects dominated by autoimmunity predictable by unlocking the breaks exerted by the immune system to maintain tolerance against self-antigen (Granier, C., et al., Rev Med Interne, 37(10):694-700 (2016)).

Therefore it is an object of the invention to provide compositions and methods to modulate the immune system by targeting different proteins involved in signal transduction of immune cells.

Another object of the invention provides antibodies and fragments thereof that block or inhibit suppressive signal transduction in immune cells.

Another object of the invention provides fusion proteins that block suppressive signal transduction in immune cells.

Another object of the invention provides methods for modulating an immune response by inhibiting suppressive signal transduction in immune cells.

### SUMMARY OF THE INVENTION

Compositions that bind to killer cell lectin-like receptor G1 (KLRG1) or its ligands are provided. The compositions can agonize or antagonize signal transduction through KLRG1. For example, some compositions either inhibit or block signal transduction through KLRG1 while other compositions induce or enhance signal transduction through KLRG1. In some embodiments, the compositions are anti-KLRG1 antibodies, KLRG1 fusion proteins, or KLRG1 ligand fusion proteins. One embodiment provides antibodies and antigen binding fragments thereof that immunospecifically bind KLRG1 and block, inhibit or reduce signal transduction through KLRG1. In another embodiment, the antibodies specifically bind to human KLRG1 under physiological conditions and enhance signal transduction through KLRG1. In certain embodiments, the antibodies bind to KLRG1 that is arrayed on the surface of a cell; arrayed on the surface of a cell at an endogenous concentration; arrayed on the surface of a live cell, and inhibits or blocks interaction of KLRG1 and a ligand of KLRG1; or a combination thereof. In one embodiment, the antibody or antigen binding fragment thereof binds to the extracellular domain of KLRG1 expressed on an immune cell including, but not limited to natural killer cells and T cells.

In another embodiment, the antibody or antigen binding fragment thereof has one or more constant domains from an immunoglobulin constant region (Fc), preferably human constant domains. The human constant domains can be IgA, IgD, IgE, IgG or IgM domains. The human IgG constant domains can be IgG1, IgG2, IgG3, or IgG4 domains.

Another embodiment provides an anti-KLRG1 antibody or antigen binding fragment thereof that is detectably labeled or has a conjugated toxin, drug, receptor, enzyme, receptor ligand. The antibody or antigen binding fragment thereof can be a monoclonal antibody, a human antibody, a chimeric antibody, a humanized antibody, or a single chain antibody.

In still other embodiments, the antibody or antigen binding fragment thereof is a monospecific, bispecific, trispecific, or multispecific antibody.

In certain embodiments, the antibody or antigen binding fragment thereof is modified so that the molecule will exhibit diminished or no Fc receptor (FcR) binding activity or is modified to exhibit enhanced antibody-dependent cell-mediated cytotoxicity (ADCC) or complement dependent cytotoxicity (CDC) activities.

Another embodiment provides a pharmaceutical composition containing an anti-KLRG1 antibody or antigen binding fragment thereof, wherein the antibody reduces or prevents binding of KLRG1 to a ligand thereof and/or reduces or prevents KLRG1-mediated signal transduction and a physiologically acceptable carrier or excipient. Preferred ligands are E-Cadherin, N-Cadherin, R-Cadherin, or a combination thereof.

One embodiment provides a monoclonal antibody produced by a hybridoma from the group consisting of K1A6, K1B9, K1F7, K2H11, K3G4, K3H11, K4C10, K4F4 K4F5, K4F5A, K7C12, K8F2, K9H1, or K10D5.

One embodiment provides an antibody, preferably a monoclonal antibody, or an antigen binding fragment thereof having a light chain with at least 50%, 60%, 70%, 80%, 85%, 90%, 95%, 99%, or 100% sequence identity with amino acid sequences that are selected from the group consisting of SEQ ID NOs: 4, 14, 24, 34, 44, 57, 67, 77, 87, 95, 105, or 122, and a heavy chain having at least 50%, 60%, 70%, 80%, 85%, 90%, 95%, 99%, or 100% sequence identity with amino acid sequences that are selected from the group consisting of SEQ ID NOs: 9, 19, 29, 39, 49, 55, 62, 72, 90, 100, 108, or 116.

One embodiment provides an antibody, preferably a monoclonal antibody, or antigen binding fragment thereof that has a light chain containing CDRs having at least 50%, 60%, 70%, 80%, 85%, 90%, 95%, 99%, or 100% sequence identity with amino acid sequences that are selected from the group consisting of SEQ ID Nos: 5, 15, 25, 35, 45, 58, 68, 78, 96, 106, or 113, SEQ ID Nos: 6, 16, 26, 36, 46, 59, 69, 79, or 97, and SEQ ID Nos: 7, 17, 27, 37, 47, 60, 70, 80, 88, 98, or 114.

One embodiment provides an antibody, preferably a monoclonal antibody, or antigen binding fragment thereof that has a heavy chain containing CDRs having at least 50%, 60%, 70%, 80%, 85%, 90%, 95%, 99%, or 100% sequence identity with amino acid sequences that are selected from the group consisting of SEQ ID Nos: 10, 20, 30, 40, 50, 63, 73, 83, 91, 101, or 117, SEQ ID Nos: 11, 21, 31, 41, 51, 64, 74, 84, 92, 102, 109, or 118, and SEQ ID NOs: 12, 22, 32, 42, 52, 65, 75, 85, 93, 103, 110, or 119.

Another embodiment provides an antibody, preferably a monoclonal antibody, or antigen binding fragment thereof that has a light chain with three CDRs having at least 50%, 60%, 70%, 80%, 85%, 90%, 95%, 99%, or 100% sequence identity with amino acid sequences that are selected from the group consisting of SEQ ID NOs: 5, 6, 7, 15, 16, 17, 25, 26, 27, 35, 36, 37, 45, 46, 46, 58, 59, 60, 68, 69, 70, 78, 79, 80, 96, 97, 98, 106, 113, or 114.

Another embodiment provides an antibody, preferably a monoclonal antibody, or antigen binding fragment thereof that has a heavy chain with three CDRs having at least 50%, 60%, 70%, 80%, 85%, 90%, 95%, 99%, or 100% sequence identity with amino acid sequences that are selected from the group consisting of SEQ ID NOs: 10, 11, 12, 20, 21, 22, 30, 31, 32, 40, 41, 42, 50, 51, 52, 63, 64, 65, 73, 74, 75, 83, 84, 85, 91, 92, 93, 101, 102, 103, 109, 100, 117, 118, or 119.

One embodiment provides an antibody, preferably a monoclonal antibody, or antigen binding fragment thereof that has a light chain containing three CDRs having at least 50%, 60%, 70%, 80%, 85%, 90%, 95%, 99%, or 100% sequence identity with amino acid sequences that are selected from the group consisting of SEQ ID NOs: 5, 6, 7, 15, 16, 17, 25, 26, 27, 35, 36, 37, 45, 46, 46, 58, 59, 60, 68, 69, 70, 78, 79, 80, 96, 97, 98, 106, 113, or 114, and a heavy chain containing three CDRs with amino acid sequences that are selected from the group consisting of SEQ ID NOs: 10, 11, 12, 20, 21, 22, 30, 31, 32, 40, 41, 42, 50, 51, 52, 63, 64, 65, 73, 74, 75, 83, 84, 85, 91, 92, 93, 101, 102, 103, 109, 100, 117, 118, or 119.

One embodiment provides a monoclonal antibody or antigen binding fragment thereof that has a light chain having at least 50%, 60%, 70%, 80%, 85%, 90%, 95%, 99%, or 100% sequence identity with SEQ ID NO:4.

Another embodiment provides a nucleic acid that encodes the light chain SEQ ID NO:4 having at least 50%, 60%, 70%, 80%, 85%, 90%, 95%, 99%, or 100% sequence identity with SEQ ID NO:8.

One embodiment provides a monoclonal antibody or antigen binding fragment thereof that has a heavy chain having at least 50%, 60%, 70%, 80%, 85%, 90%, 95%, 99%, or 100% sequence identity with SEQ ID NO:9.

Another embodiment provides a nucleic acid that encodes the heavy chain SEQ ID NO:9 having at least 50%, 60%, 70%, 80%, 85%, 90%, 95%, 99%, or 100% sequence identity with SEQ ID NO:13.

One embodiment provides an antibody, preferably a monoclonal antibody, or antigen binding fragment thereof that has a light chain containing CDRs having at least 50%, 60%, 70%, 80%, 85%, 90%, 95%, 99%, or 100% sequence identity to SEQ ID Nos: 5, 6, and 7.

One embodiment provides an antibody, preferably a monoclonal antibody, or an antigen binding fragment thereof having a heavy chain containing CDRs having at least 50%, 60%, 70%, 80%, 85%, 90%, 95%, 99%, or 100% sequence identity to SEQ ID Nos: 10, 11, and 12.

One embodiment provides an antibody, preferably a monoclonal antibody, or antigen binding fragment thereof that has a light chain containing CDRs according to SEQ ID Nos: 5, 6, and 7 and a heavy chain containing CDRs according to SEQ ID Nos: 10, 11, and 12.

One embodiment provides an antibody, preferably a monoclonal antibody, or an antigen binding fragment thereof having a light chain at least 50%, 60%, 70%, 80%, 85%, 90%, 95%, 99%, or 100% sequence identity to SEQ ID NO:4 and a heavy chain having at least 50%, 60%, 70%, 80%, 85%, 90%, 95%, 99%, or 100% sequence identity to SEQ ID NO:9.

One embodiment provides a monoclonal antibody or antigen binding fragment thereof that has a light chain having at least 50%, 60%, 70%, 80%, 85%, 90%, 95%, 99%, or 100% sequence identity with SEQ ID NO:14.

One embodiment provides an antibody, preferably a monoclonal antibody, or antigen binding fragment thereof that has a light chain containing CDRs according to SEQ ID Nos: 15, 16, and 17.

One embodiment provides a monoclonal antibody or antigen binding fragment thereof that has a heavy chain having at least 50%, 60%, 70%, 80%, 85%, 90%, 95%, 99%, or 100% sequence identity with SEQ ID NO:19.

One embodiment provides an antibody, preferably a monoclonal antibody, or antigen binding fragment thereof that has a heavy chain containing CDRs according to SEQ ID Nos: 20, 21, and 22.

One embodiment provides a monoclonal antibody, or antigen binding fragment thereof having a light chain CDRs at least 50%, 60%, 70%, 80%, 85%, 90%, 95%, 99%, or 100% sequence identity to SEQ ID Nos: 15, 16, and 17 and heavy chain CDRs at least 50%, 60%, 70%, 80%, 85%, 90%, 95%, 99%, or 100% sequence identity to SEQ ID Nos: 20, 21, and 22.

One embodiment provides an antibody, preferably a monoclonal antibody, or an antigen binding fragment thereof that has a light chain having at least 50%, 60%, 70%, 80%, 85%, 90%, 95%, 99%, or 100% sequence identity to SEQ ID NO:14 and a heavy chain having at least 50%, 60%, 70%, 80%, 85%, 90%, 95%, 99%, or 100% sequence identity to SEQ ID NO:19.

Another embodiment provides an antibody light chain having at least 50%, 60%, 70%, 80%, 85%, 90%, 95%, 99%, or 100% sequence identity to SEQ ID NO:24.

One embodiment provides an antibody light chain having complementarity-determining regions (CDRs) having at least 50%, 60%, 70%, 80%, 85%, 90%, 95%, 99%, or 100% sequence identity with amino acid sequences according to SEQ ID NOs:25, 26, and 27.

One embodiment provides an antibody heavy chain having at least 50%, 60%, 70%, 80%, 85%, 90%, 95%, 99%, or 100% sequence identity to SEQ ID NO:29.

Another embodiment provides an antibody heavy chain that has CDRs having at least 50%, 60%, 70%, 80%, 85%, 90%, 95%, 99%, or 100% sequence identity with amino acid sequences according to SEQ ID NOs:30, 31, and 32.

One embodiment provides a monoclonal antibody, or antigen binding fragment thereof comprising light chain CDRs at least 50%, 60%, 70%, 80%, 85%, 90%, 95%, 99%, or 100% sequence identity to SEQ ID Nos: 25, 26, and 27 and heavy chain CDRs at least 50%, 60%, 70%, 80%, 85%, 90%, 95%, 99%, or 100% sequence identity to SEQ ID Nos: 30, 31, and 32.

One embodiment provides an antibody, or an antigen binding fragment thereof that has a light chain having at least 50%, 60%, 70%, 80%, 85%, 90%, 95%, 99%, or 100% sequence identity to SEQ ID NO:24 and a heavy chain having at least 50%, 60%, 70%, 80%, 85%, 90%, 95%, 99%, or 100% sequence identity to SEQ ID NO:29.

Another embodiment provides an antibody light chain having at least 50%, 60%, 70%, 80%, 85%, 90%, 95%, 99%, or 100% sequence identity to SEQ ID NO:34.

One embodiment provides an antibody light chain that has complementarity-determining regions (CDRs) having at least 50%, 60%, 70%, 80%, 85%, 90%, 95%, 99%, or 100% sequence identity with amino acid sequences according to SEQ ID NOs:35, 36, and 37.

One embodiment provides an antibody heavy chain having at least 50%, 60%, 70%, 80%, 85%, 90%, 95%, 99%, or 100% sequence identity to SEQ ID NO:39.

One embodiment provides an antibody heavy chain having CDRs with at least 50%, 60%, 70%, 80%, 85%, 90%, 95%, 99%, or 100% sequence identity with amino acid sequences according to SEQ ID NOs:40, 41, and 42.

Another embodiment provides a monoclonal antibody, or antigen binding fragment thereof comprising light chain CDRs having at least 50%, 60%, 70%, 80%, 85%, 90%, 95%, 99%, or 100% sequence identity to SEQ ID Nos: 35, 36, and 37 and heavy chain CDRs according to SEQ ID Nos: 40, 41, and 42.

One embodiment provides an antibody, or an antigen binding fragment thereof that has a light chain having at least 50%, 60%, 70%, 80%, 85%, 90%, 95%, 99%, or 100% sequence identity to SEQ ID NO:34 and a heavy chain having at least 50%, 60%, 70%, 80%, 85%, 90%, 95%, 99%, or 100% sequence identity to SEQ ID NO:39.

One embodiment provides an antibody light chain having at least 50%, 60%, 70%, 80%, 85%, 90%, 95%, 99%, or 100% sequence identity to SEQ ID NO:44.

One embodiment provides an antibody light chain that has complementarity-determining regions (CDRs) having at least 50%, 60%, 70%, 80%, 85%, 90%, 95%, 99%, or 100% sequence identity with amino acid sequences according to SEQ ID NOs:45, 46, and 47.

Another embodiment provides an antibody heavy chain having at least 50%, 60%, 70%, 80%, 85%, 90%, 95%, 99%, or 100% sequence identity to SEQ ID NO:49.

One embodiment provides an antibody heavy chain having CDRs having at least 50%, 60%, 70%, 80%, 85%, 90%, 95%, 99%, or 100% sequence identity with amino acid sequences according to SEQ ID NOs:50, 51, and 52.

One embodiment provides a monoclonal antibody, or antigen binding fragment thereof that has light chain CDRs having at least 50%, 60%, 70%, 80%, 85%, 90%, 95%, 99%, or 100% sequence identity to SEQ ID Nos: 45, 46, and 47 and heavy chain CDRs according to SEQ ID Nos: 50, 51, and 52.

Another embodiment provides an antibody, or an antigen binding fragment thereof that has a light chain having at least 50%, 60%, 70%, 80%, 85%, 90%, 95%, 99%, or 100% sequence identity to SEQ ID NO:44 and a heavy chain having at least 50%, 60%, 70%, 80%, 85%, 90%, 95%, 99%, or 100% sequence identity to SEQ ID NO:49.

One embodiment provides an antibody heavy chain having at least 50%, 60%, 70%, 80%, 85%, 90%, 95%, 99%, or 100% sequence identity to SEQ ID NO:55.

One embodiment provides an antibody, or an antigen binding fragment thereof that has a light chain having at least 50%, 60%, 70%, 80%, 85%, 90%, 95%, 99%, or 100% sequence identity to SEQ ID NO:24 and a heavy chain having at least 50%, 60%, 70%, 80%, 85%, 90%, 95%, 99%, or 100% sequence identity to SEQ ID NO:55.

Another embodiment provides an antibody light chain having at least 50%, 60%, 70%, 80%, 85%, 90%, 95%, 99%, or 100% sequence identity to SEQ ID NO:57.

One embodiment provides an antibody light chain having complementarity-determining regions (CDRs) having at least 50%, 60%, 70%, 80%, 85%, 90%, 95%, 99%, or 100% sequence identity with amino acid sequences according to SEQ ID NOs:58, 59, and 60.

One embodiment provides an antibody heavy chain having at least 50%, 60%, 70%, 80%, 85%, 90%, 95%, 99%, or 100% sequence identity to SEQ ID NO:62.

One embodiment provides an antibody heavy chain having CDRs having at least 50%, 60%, 70%, 80%, 85%, 90%, 95%, 99%, or 100% sequence identity with amino acid sequences according to SEQ ID NOs:63, 64, and 65.

Another embodiment provides a monoclonal antibody, or antigen binding fragment thereof having light chain CDRs according to SEQ ID Nos: 58, 59, and 60 and heavy chain CDRs according to SEQ ID Nos: 63, 64, and 65.

One embodiment provides an antibody, or an antigen binding fragment thereof that has a light chain having at least 50%, 60%, 70%, 80%, 85%, 90%, 95%, 99%, or 100% sequence identity to SEQ ID NO:57 and a heavy chain having at least 50%, 60%, 70%, 80%, 85%, 90%, 95%, 99%, or 100% sequence identity to SEQ ID NO:62.

One embodiment provides an antibody light chain having at least 50%, 60%, 70%, 80%, 85%, 90%, 95%, 99%, or 100% sequence identity to SEQ ID NO:67.

One embodiment provides an antibody light chain having complementarity-determining regions (CDRs) having at least 50%, 60%, 70%, 80%, 85%, 90%, 95%, 99%, or 100% sequence identity with amino acid sequences according to SEQ ID NOs:68, 69, and 70.

Another embodiment provides an antibody heavy chain having at least 50%, 60%, 70%, 80%, 85%, 90%, 95%, 99%, or 100% sequence identity to SEQ ID NO:72.

One embodiment provides an antibody heavy chain having CDRs having at least 50%, 60%, 70%, 80%, 85%, 90%, 95%, 99%, or 100% sequence identity with amino acid sequences according to SEQ ID NOs:73, 74, and 75.

One embodiment provides a monoclonal antibody, or antigen binding fragment thereof that has light chain CDRs having at least 50%, 60%, 70%, 80%, 85%, 90%, 95%, 99%, or 100% sequence identity according to SEQ ID Nos: 68, 69, and 70 and heavy chain CDRs according to SEQ ID Nos: 73, 74, and 75.

One embodiment provides an antibody, or an antigen binding fragment thereof that has a light chain having at least 50%, 60%, 70%, 80%, 85%, 90%, 95%, 99%, or 100% sequence identity to SEQ ID NO:67 and a heavy chain having at least 50%, 60%, 70%, 80%, 85%, 90%, 95%, 99%, or 100% sequence identity to SEQ ID NO:72.

Another embodiment provides an antibody light chain having at least 50%, 60%, 70%, 80%, 85%, 90%, 95%, 99%, or 100% sequence identity to SEQ ID NO:77.

One embodiment provides an antibody light chain having complementarity-determining regions (CDRs) having at least 50%, 60%, 70%, 80%, 85%, 90%, 95%, 99%, or 100% sequence identity with amino acid sequences according to SEQ ID NOs:78, 79, and 80.

One embodiment provides an antibody heavy chain having at least 50%, 60%, 70%, 80%, 85%, 90%, 95%, 99%, or 100% sequence identity to SEQ ID NO:82.

One embodiment provides an antibody heavy chain that has CDRs having at least 50%, 60%, 70%, 80%, 85%, 90%, 95%, 99%, or 100% sequence identity with amino acid sequences according to SEQ ID NOs:83, 84, and 85.

Another embodiment provides a monoclonal antibody, or antigen binding fragment thereof having light chain CDRs having at least 50%, 60%, 70%, 80%, 85%, 90%, 95%, 99%, or 100% sequence identity according to SEQ ID Nos:78, 79, and 80 and heavy chain CDRs according to SEQ ID Nos: 83, 84, and 85.

One embodiment provides an antibody, or an antigen binding fragment thereof that has a light chain having at least 50%, 60%, 70%, 80%, 85%, 90%, 95%, 99%, or 100% sequence identity to SEQ ID NO:77 and a heavy chain having at least 50%, 60%, 70%, 80%, 85%, 90%, 95%, 99%, or 100% sequence identity to SEQ ID NO:22.

One embodiment provides an antibody light chain having at least 50%, 60%, 70%, 80%, 85%, 90%, 95%, 99%, or 100% sequence identity to SEQ ID NO:87.

One embodiment provides an antibody light chain having complementarity-determining regions (CDRs) having at least 50%, 60%, 70%, 80%, 85%, 90%, 95%, 99%, or 100% sequence identity with amino acid sequences according to SEQ ID NOs:58, 59, and 88.

Another embodiment provides an antibody heavy chain having at least 50%, 60%, 70%, 80%, 85%, 90%, 95%, 99%, or 100% sequence identity to SEQ ID NO:90.

One embodiment provides an antibody heavy chain having CDRs having at least 50%, 60%, 70%, 80%, 85%, 90%, 95%, 99%, or 100% sequence identity with amino acid sequences according to SEQ ID NOs:91, 92, and 93.

One embodiment provides a monoclonal antibody, or antigen binding fragment thereof having light chain CDRs having at least 50%, 60%, 70%, 80%, 85%, 90%, 95%, 99%, or 100% sequence identity according to SEQ ID Nos:58, 59, and 88 and heavy chain CDRs according to SEQ ID Nos: 91, 92, and 93.

One embodiment provides an antibody, or an antigen binding fragment thereof that has a light chain having at least 50%, 60%, 70%, 80%, 85%, 90%, 95%, 99%, or 100% sequence identity to SEQ ID NO:87 and a heavy chain having at least 50%, 60%, 70%, 80%, 85%, 90%, 95%, 99%, or 100% sequence identity to SEQ ID NO:90.

Another embodiment provides an antibody light chain having at least 50%, 60%, 70%, 80%, 85%, 90%, 95%, 99%, or 100% sequence identity to SEQ ID NO:95.

One embodiment provides an antibody light chain having complementarity-determining regions (CDRs) having at least 50%, 60%, 70%, 80%, 85%, 90%, 95%, 99%, or 100% sequence identity with amino acid sequences according to SEQ ID NOs:96, 97, and 98.

One embodiment provides an antibody heavy chain having at least 50%, 60%, 70%, 80%, 85%, 90%, 95%, 99%, or 100% sequence identity to SEQ ID NO:100.

One embodiment provides an antibody heavy chain that has CDRs having at least 50%, 60%, 70%, 80%, 85%, 90%, 95%, 99%, or 100% sequence identity with amino acid sequences according to SEQ ID NOs:101, 102, and 103.

Another embodiment provides a monoclonal antibody, or antigen binding fragment thereof having light chain CDRs having at least 50%, 60%, 70%, 80%, 85%, 90%, 95%, 99%, or 100% sequence identity according to SEQ ID Nos: 96, 97, and 98 and heavy chain CDRs according to SEQ ID Nos: 101, 102, and 103.

One embodiment provides an antibody, or an antigen binding fragment thereof that has a light chain having at least 50%, 60%, 70%, 80%, 85%, 90%, 95%, 99%, or 100% sequence identity to SEQ ID NO:95 and a heavy chain having at least 50%, 60%, 70%, 80%, 85%, 90%, 95%, 99%, or 100% sequence identity to SEQ ID NO:100.

One embodiment provides an antibody light chain having at least 50%, 60%, 70%, 80%, 85%, 90%, 95%, 99%, or 100% sequence identity to SEQ ID NO:105.

One embodiment provides an antibody light chain having complementarity-determining regions (CDRs) having at least 50%, 60%, 70%, 80%, 85%, 90%, 95%, 99%, or 100% sequence identity with amino acid sequences according to SEQ ID NOs:106, 26, and 7.

Another embodiment provides an antibody heavy chain having at least 50%, 60%, 70%, 80%, 85%, 90%, 95%, 99%, or 100% sequence identity to SEQ ID NO:108.

One embodiment provides an antibody heavy chain having CDRs having at least 50%, 60%, 70%, 80%, 85%, 90%, 95%, 99%, or 100% sequence identity with amino acid sequences according to SEQ ID NOs:101, 109, and 110.

One embodiment provides a monoclonal antibody, or antigen binding fragment thereof that has light chain CDRs having at least 50%, 60%, 70%, 80%, 85%, 90%, 95%, 99%, or 100% sequence identity according to SEQ ID Nos: 106, 26, and 7 and heavy chain CDRs according to SEQ ID Nos: 101, 109, and 110.

One embodiment provides an antibody, or an antigen binding fragment thereof that has a light chain having at least 50%, 60%, 70%, 80%, 85%, 90%, 95%, 99%, or 100% sequence identity to SEQ ID NO:105 and a heavy chain having at least 50%, 60%, 70%, 80%, 85%, 90%, 95%, 99%, or 100% sequence identity to SEQ ID NO:108.

One embodiment provides an antibody light chain having at least 50%, 60%, 70%, 80%, 85%, 90%, 95%, 99%, or 100% sequence identity to SEQ ID NO:112.

Another embodiment provides an antibody light chain comprising complementarity-determining regions (CDRs) having at least 50%, 60%, 70%, 80%, 85%, 90%, 95%, 99%, or 100% sequence identity with amino acid sequences according to SEQ ID NOs:113, 59, and 114.

One embodiment provides an antibody heavy chain having at least 50%, 60%, 70%, 80%, 85%, 90%, 95%, 99%, or 100% sequence identity to SEQ ID NO:116.

One embodiment provides an antibody heavy chain having CDRs having at least 50%, 60%, 70%, 80%, 85%, 90%, 95%, 99%, or 100% sequence identity with amino acid sequences according to SEQ ID NOs:117, 118, and 119.

One embodiment provides a monoclonal antibody, or antigen binding fragment thereof having light chain CDRs having at least 50%, 60%, 70%, 80%, 85%, 90%, 95%, 99%, or 100% sequence identity according to SEQ ID Nos: 113, 59, and 114 and heavy chain CDRs according to SEQ ID Nos: 117, 118, and 119.

Another embodiment provides an antibody, or an antigen binding fragment thereof that has a light chain having at least 50%, 60%, 70%, 80%, 85%, 90%, 95%, 99%, or 100% sequence identity to SEQ ID NO:112 and a heavy chain having at least 50%, 60%, 70%, 80%, 85%, 90%, 95%, 99%, or 100% sequence identity to SEQ ID NO:116.

One embodiment provides an antibody light chain having at least 50%, 60%, 70%, 80%, 85%, 90%, 95%, 99%, or 100% sequence identity to SEQ ID NO:112.

One embodiment provides an antibody light chain having complementarity-determining regions (CDRs) having at least 50%, 60%, 70%, 80%, 85%, 90%, 95%, 99%, or 100% sequence identity with amino acid sequences according to SEQ ID NOs:113, 59, and 114.

One embodiment provides an antibody heavy chain having at least 50%, 60%, 70%, 80%, 85%, 90%, 95%, 99%, or 100% sequence identity to SEQ ID NO:116.

Another embodiment provides an antibody heavy chain having CDRs having at least 50%, 60%, 70%, 80%, 85%, 90%, 95%, 99%, or 100% sequence identity with amino acid sequences according to SEQ ID NOs:117, 118, and 119.

One embodiment provides a monoclonal antibody, or antigen binding fragment thereof having light chain CDRs having at least 50%, 60%, 70%, 80%, 85%, 90%, 95%, 99%, or 100% sequence identity according to SEQ ID Nos: 113, 59, and 114 and heavy chain CDRs according to SEQ ID Nos: 117, 118, and 119.

One embodiment provides an antibody, or an antigen binding fragment thereof that has a light chain having at least 50%, 60%, 70%, 80%, 85%, 90%, 95%, 99%, or 100% sequence identity to SEQ ID NO:112 and a heavy chain having at least 50%, 60%, 70%, 80%, 85%, 90%, 95%, 99%, or 100% sequence identity to SEQ ID NO:116.

Another embodiment provides an anti-KLRG1 antibody preferably a monoclonal antibody, or an antigen binding fragment thereof having a light chain variable domain having at least 50%, 60%, 70%, 80%, 85%, 90%, 95%, 99%, or 100% sequence identity to any one of SEQ ID NOs:121, 122, 123, 124, or 125, and a heavy chain variable domain having at least 50%, 60%, 70%, 80%, 85%, 90%, 95%, 99%, or 100% sequence identity to any one of SEQ ID NOs:132, 133, 134, 135, or 136.

One embodiment provides an anti-KLRG1 antibody or antigen binding fragment thereof having a light chain having an amino acid sequence according to any one of SEQ ID NOs:126, 127, 128, or 129, and a heavy chain having an amino acid sequence according to any one of SEQ ID NOs: 137, 139, 141, 143, or 145.

Another embodiment provides an anti-KLRG1 antibody or antigen binding fragment thereof having a light chain having an amino acid sequence according to any one of SEQ ID NOs: 126, 127, 128, or 129, and a heavy chain having an amino acid sequence according to any one of SEQ ID NOs: 138, 140, 142, 144, or 146.

One embodiment provides an anti-KLRG1 antibody or antigen binding fragment thereof having two light chains and two heavy chains, wherein the two light chains include a polypeptide selected from the group consisting of SEQ ID NO: 126, 127, 128, or 129, or a variant thereof having at least 50%, 60%, 70%, 80%, 85%, 90%, 95%, 99%, or more sequence identity to SEQ ID NO: 126, 127, 128, or 129, and the two heavy chains include a polypeptide selected from the group consisting of SEQ ID NO: 137, 139, 141, 143, or 145, or a variant thereof having at least 50%, 60%, 70%, 80%, 85%, 90%, 95%, 99%, or more sequence identity to SEQ ID NO:137, 139, 141, 143, or 145, and wherein the antibody or antigen binding fragment thereof binds to KLRG1.

Another embodiment provides an antibody or antigen binding fragment thereof having two light chains and two heavy chains, wherein the two light chains include a polypeptide selected from the group consisting of SEQ ID NO: 126, 127, 128, or 129, or a variant thereof having at least 50%, 60%, 70%, 80%, 85%, 90%, 95%, 99%, or more sequence identity to SEQ ID NO: 126, 127, 128, or 129, and the two heavy chains include a polypeptide selected from the group consisting of SEQ ID NO: 138, 140, 142, 144, or 146, or a variant thereof having at least 50%, 60%, 70%, 80%, 85%, 90%, 95%, 99%, or more sequence identity to SEQ ID NO: 138, 140, 142, 144, or 146, and wherein the antibody or antigen binding fragment thereof binds to KLRG1.

Yet another embodiment provides a method of treating a subject in need thereof by administering to the subject an effective amount of the pharmaceutical composition containing a KLRG1 binding molecule to the subject in an amount effective to inhibit or block signal transduction through KLRG1 on immune cells. In certain embodiments the subject has cancer or an infectious disease. The KLRG1 binding molecule can be an antibody or antigen binding fragment thereof increases an immune response, retards or prevents tumor growth, inhibits immune suppression, eliminate tumors, reduces or reverses T cell suppression, or a combination thereof. In other embodiments the method includes administering to the subject a second therapeutic agent.

Still another embodiment provides a method of detection or diagnosis of a disease, disorder or infection, including: (a) assaying the expression of KLRG1 in cells or in a tissue sample of a subject using an anti-KLRG1 antibody or antigen binding fragment thereof and (b) comparing the level of the KLRG1 with a control level, wherein an increase in the assayed level of KLRG1 compared to the control level is indicative of the disease, disorder or infection.

Another embodiment provides a method for monitoring the progression of a disease, disorder or infection, including: (a) assaying the expression of KLRG1 in cells or in a tissue sample of a subject obtained at a first time point and later time point using an anti-KLRG1 antibody or antigen binding fragment thereof; and (b) comparing the level of expression of KLRG1 in the cells or in the tissue sample of the subject at the first and later times points, wherein an increase in the assayed level of KLRG1 at the later time point compared to the first time point is indicative of the progression of disease, disorder or infection.

Still another embodiment provides a method for monitoring a response to a treatment, including: (a) assaying the expression of KLRG1 in cells or in a tissue sample of a subject prior and after the treatment using an anti-KLRG1 antibody or antigen binding fragment thereof; and (b) comparing the level of KLRG1 over time, whereby a decrease in the assayed level of KLRG1 after treatment compared to the level of KLRG1 prior to treatment is indicative of a favorable response to the treatment.

Another embodiment provides a method of reducing T cell and/or NK cell suppression in a subject in need thereof by administering to the subject an effective amount of the pharmaceutical composition containing an anti-KLRG1 antibody or binding fragment thereof to the subject in an amount effect to inhibit or block signal transduction through KLRG1 on immune cells.

Another embodiment provides a method of increasing cytokine production in T cells in a subject in need thereof by administering to the subject an effective amount of the pharmaceutical composition containing an anti-KLRG1 antibody or fragment thereof. In some embodiments, the cytokine is IL-2 and the T cells are CD4+.

Still another embodiment provides a fusion protein having a first and second fusion partner, wherein the first fusion partner has all or part of a KLRG1 polypeptide or fragment or variant thereof. In other embodiments, the first fusion partner includes a ligand of KLRG1. The second fusion partner can be an Fc domain. The Fc domain can be derived from IgG1. In one embodiment the first fusion partner and second fusion partner are linked by a linker. The linker can be a hinge domain or fragment thereof. In some embodiments the KLRG1 polypeptide includes all or part of the KLRG1 extracellular domain. The fusion protein of any can have a leader sequence.

Still another embodiment provides a pharmaceutical composition contain a KLRG1 fusion protein and a physiologically acceptable carrier or excipient in an amount effective to inhibit, reduce or block signal transduction through KLRG1 on immune cells.

Another embodiment provides a method for treating a subject in need thereof by administering to the subject an anti-KLRG1 antibody or a KLRG1 fusion protein in an amount effective to deplete KLRG1+ cells in the subject. Typically the subject has or has been diagnosed as having cancer.

Kits containing the disclosed anti-KLRG1 antibodies and KLRG1 fusion proteins are also provided.

### BRIEF DESCRIPTION OF THE DRAWINGS

FIG. 1 shows the amino acid sequence alignment of human and mouse KLRG1 extracellular domains.

### DETAILED DESCRIPTION OF THE INVENTION

### I. Definitions

As used herein, a molecule is said to be able to “immunospecifically bind” a second molecule if such binding exhibits the specificity and affinity of an antibody to its cognate antigen. Antibodies are said to be capable of immunospecifically binding to a target region or conformation (“epitope”) of an antigen if such binding involves the antigen recognition site of the immunoglobulin molecule. An antibody that immunospecifically binds to a particular antigen may bind to other antigens with lower affinity if the other antigen has some sequence or conformational similarity that is recognized by the antigen recognition site as determined by, e.g., immunoassays, BIACORE® assays, or other assays known in the art, but would not bind to a totally unrelated antigen. Preferably, however, antibodies (and their antigen binding fragments) will not cross-react with other antigens. Antibodies may also bind to other molecules in a way that is not immunospecific, such as to FcR receptors, by virtue of binding domains in other regions/domains of the molecule that do not involve the antigen recognition site, such as the Fc region.

As used herein, a molecule is said to “physiospecifically bind” a second molecule if such binding exhibits the specificity and affinity of a receptor to its cognate binding ligand. A molecule can be capable of physiospecifically binding to more than one other molecule.

As used herein, the term “antibody” is intended to denote an immunoglobulin molecule that possesses a “variable region” antigen recognition site. The term “variable region” is intended to distinguish such domain of the immunoglobulin from domains that are broadly shared by antibodies (such as an antibody Fc domain). The variable region includes a “hypervariable region” whose residues are responsible for antigen binding. The hypervariable region includes amino acid residues from a “Complementarity Determining Region” or “CDR” (i.e., typically at approximately residues 24-34 (L1), 50-56 (L2) and 89-97 (L3) in the light chain variable domain and at approximately residues 27-35 (H1), 50-65 (H2) and 95-102 (H3) in the heavy chain variable domain; Kabat et al., Sequences of Proteins of Immunological Interest, 5th Ed. Public Health Service, National Institutes of Health, Bethesda, Md. (1991)) and/or those residues from a “hypervariable loop” (i.e., residues 26-32 (L1), 50-52 (L2) and 91-96 (L3) in the light chain variable domain and 26-32 (H1), 53-55 (H2) and 96-101 (H3) in the heavy chain variable domain; CHOTHIA et al., J Mol Biol, 196:901-917 (1987). “Framework Region” or “FR” residues are those variable domain residues other than the hypervariable region residues as herein defined. The term antibody includes monoclonal antibodies, multi-specific antibodies, human antibodies, humanized antibodies, synthetic antibodies, chimeric antibodies, camelized antibodies (See e.g., Muyldermans et al., Trends Biochem Sci, 26:230 (2001); Nuttall et al., Cur Pharm Biotech, 1:253 (2000); Reichmann et al., J Immunol Meth, 231:25 (1999); International Publication Nos. WO 94/04678 and WO 94/25591; U.S. Pat. No. 6,005,079), single-chain Fvs (scFv) (see, e.g., see Pluckthun in The Pharmacology of Monoclonal Antibodies, Vol. 113, Rosenburg and Moore Eds. Springer-Verlag, New York, pp. 269-315 (1994)), single chain antibodies, disulfide-linked Fvs (sdFv), intrabodies, and anti-idiotypic (anti-Id) antibodies (including, e.g., anti-Id and anti-anti-Id antibodies to antibodies). In particular, such antibodies include immunoglobulin molecules of any type (e.g., IgG, IgE, IgM, IgD, IgA and IgY), class (e.g., IgG₁, IgG₂, IgG₃, IgG₄, IgA₁ and IgA₂) or subclass.

As used herein, the term “antigen binding fragment” of an antibody refers to one or more portions of an antibody that contain the antibody's Complementarity Determining Regions (“CDRs”) and optionally the framework residues that include the antibody's “variable region” antigen recognition site, and exhibit an ability to immunospecifically bind antigen. Such fragments include Fab′, F(ab′)₂, Fv, single chain (ScFv), and mutants thereof, naturally occurring variants, and fusion proteins including the antibody's “variable region” antigen recognition site and a heterologous protein (e.g., a toxin, an antigen recognition site for a different antigen, an enzyme, a receptor or receptor ligand, etc.).

As used herein, the term “fragment” refers to a peptide or polypeptide including an amino acid sequence of at least 5 contiguous amino acid residues, at least 10 contiguous amino acid residues, at least 15 contiguous amino acid residues, at least 20 contiguous amino acid residues, at least 25 contiguous amino acid residues, at least 40 contiguous amino acid residues, at least 50 contiguous amino acid residues, at least 60 contiguous amino residues, at least 70 contiguous amino acid residues, at least 80 contiguous amino acid residues, at least 90 contiguous amino acid residues, at least 100 contiguous amino acid residues, at least 125 contiguous amino acid residues, at least 150 contiguous amino acid residues, at least 175 contiguous amino acid residues, at least 200 contiguous amino acid residues, or at least 250 contiguous amino acid residues.

As used herein the term “modulate” relates to a capacity to alter an effect, result, or activity (e.g., signal transduction). Such modulation can agonistic or antagonistic. Antagonistic modulation can be partial (i.e., attenuating, but not abolishing) or it can completely abolish such activity (e.g., neutralizing). Modulation can include internalization of a receptor following binding of an antibody or a reduction in expression of a receptor on the target cell. Agonistic modulation can enhance or otherwise increase or enhance an activity (e.g., signal transduction). In a still further embodiment, such modulation can alter the nature of the interaction between a ligand and its cognate receptor so as to alter the nature of the elicited signal transduction. For example, the molecules can, by binding to the ligand or receptor, alter the ability of such molecules to bind to other ligands or receptors and thereby alter their overall activity. Preferably, such modulation will provide at least a 10% change in a measurable immune system activity, more preferably, at least a 50% change in such activity, or at least a 2-fold, 5-fold, 10-fold, or still more preferably, at least a 100-fold change in such activity.

The term “substantially,” as used in the context of binding or exhibited effect, is intended to denote that the observed effect is physiologically or therapeutically relevant. Thus, for example, a molecule is able to substantially block an activity of a ligand or receptor if the extent of blockage is physiologically or therapeutically relevant (for example if such extent is greater than 60% complete, greater than 70% complete, greater than 75% complete, greater than 80% complete, greater than 85% complete, greater than 90% complete, greater than 95% complete, or greater than 97% complete). Similarly, a molecule is said to have substantially the same immunospecificity and/or characteristic as another molecule, if such immunospecificities and characteristics are greater than 60% identical, greater than 70% identical, greater than 75% identical, greater than 80% identical, greater than 85% identical, greater than 90% identical, greater than 95% identical, or greater than 97% identical).

As used herein, the “co-stimulatory” signals encompass positive co-stimulatory signals (e.g., signals that result in enhancing an activity) and negative co-stimulatory signals (e.g., signals that result in inhibiting an activity).

As used herein, the term “derivative” refers to an antibody or antigen-binding fragment thereof that immunospecifically binds to the same target of a parent or reference antibody but which differs in amino acid sequence from the parent or reference antibody or antigen binding fragment thereof by including one, two, three, four, five or more amino acid substitutions, additions, deletions or modifications relative to the parent or reference antibody or antigen binding fragment thereof. Preferably such derivatives will have substantially the same immunospecificity and/or characteristics, or the same immunospecificity and characteristics as the parent or reference antibody or antigen binding fragment thereof. The amino acid substitutions or additions of such derivatives can include naturally occurring (i.e., DNA-encoded) or non-naturally occurring amino acid residues. The term “derivative” encompasses, for example, chimeric or humanized variants, as well as variants having altered CH₁, hinge, CH₂, CH₃ or CH₄ regions, so as to form, for example antibodies, etc., having variant Fc regions that exhibit enhanced or impaired effector or binding characteristics.

As used herein, a “chimeric antibody” is a molecule in which different portions of the antibody are derived from different immunoglobulin molecules such as antibodies having a variable region derived from a non-human antibody and a human immunoglobulin constant region.

As used herein, the term “humanized antibody” refers to an immunoglobulin including a human framework region and one or more CDR's from a non-human (usually a mouse or rat) immunoglobulin. The non-human immunoglobulin providing the CDR's is called the “donor” and the human immunoglobulin providing the framework is called the “acceptor.” Constant regions need not be present, but if they are, they should be substantially identical to human immunoglobulin constant regions, i.e., at least about 85-99%, preferably about 95% or more identical. Hence, all parts of a humanized immunoglobulin, except possibly the CDR's, are substantially identical to corresponding parts of natural human immunoglobulin sequences. A humanized antibody is an antibody including a humanized light chain and a humanized heavy chain immunoglobulin. For example, a humanized antibody would not encompass a typical chimeric antibody, because, e.g., the entire variable region of a chimeric antibody is non-human.

As used herein, the term “endogenous concentration” refers to the level at which a molecule is natively expressed (i.e., in the absence of expression vectors or recombinant promoters) by a cell (which cell can be a normal cell, a cancer cell or an infected cell).

As used herein, the terms “treat,” “treating,” “treatment” and “therapeutic use” refer to the elimination, reduction or amelioration of one or more symptoms of a disease or disorder exacerbated by KLRG1 or a ligand thereof.

As used herein, a “therapeutically effective amount” refers to that amount of a therapeutic agent sufficient to mediate a clinically relevant elimination, reduction or amelioration of such symptoms. An effect is clinically relevant if its magnitude is sufficient to impact the health or prognosis of a recipient subject. A therapeutically effective amount may refer to the amount of therapeutic agent sufficient to delay or minimize the onset of disease, e.g., delay or minimize the spread of cancer. A therapeutically effective amount may also refer to the amount of the therapeutic agent that provides a therapeutic benefit in the treatment or management of a disease.

As used herein, the term “prophylactic agent” refers to an agent that can be used in the prevention of a disorder or disease prior to the detection of any symptoms of such disorder or disease. A “prophylactically effective” amount is the amount of prophylactic agent sufficient to mediate such protection. A prophylactically effective amount may also refer to the amount of the prophylactic agent that provides a prophylactic benefit in the prevention of disease.

As used herein, the term “cancer” refers to a neoplasm or tumor resulting from abnormal uncontrolled growth of cells. As used herein, cancer explicitly includes leukemias and lymphomas. The term “cancer” refers to a disease involving cells that have the potential to metastasize to distal sites and exhibit phenotypic traits that differ from those of non-cancer cells, for example, formation of colonies in a three-dimensional substrate such as soft agar or the formation of tubular networks or web-like matrices in a three-dimensional basement membrane or extracellular matrix preparation. Non-cancer cells do not form colonies in soft agar and form distinct sphere-like structures in three-dimensional basement membrane or extracellular matrix preparations.

As used herein, an “immune cell” refers to any cell from the hemopoietic origin including, but not limited to, T cells, B cells, natural killer cells, NKT cells, monocytes, dendritic cells, macrophages, neutrophils, eosinophils, basophils, mast cells, innate lymphoid cells (ILCs), and myeloid derived suppressor cells (MDSCs).

As used herein, “valency” refers to the number of binding sites available per molecule.

As used herein, the terms “immunologic,” “immunological” or “immune” response is the development of a beneficial humoral (antibody mediated) and/or a cellular (mediated by antigen-specific T cells or their secretion products) response directed against a peptide in a recipient patient. Such a response can be an active response induced by administration of immunogen or a passive response induced by administration of antibody or primed T-cells. A cellular immune response is elicited by the presentation of polypeptide epitopes in association with Class I or Class II MHC molecules to activate antigen-specific CD4⁺ T helper cells and/or CD8⁺ cytotoxic T cells. The response may also involve activation of monocytes, macrophages, NK cells, basophils, dendritic cells, astrocytes, microglia cells, eosinophils, activation or recruitment of neutrophils or other components of innate immunity. The presence of a cell-mediated immunological response can be determined by proliferation assays (CD4⁺ T cells) or CTL (cytotoxic T lymphocyte) assays. The relative contributions of humoral and cellular responses to the protective or therapeutic effect of an immunogen can be distinguished by separately isolating antibodies and T-cells from an immunized syngeneic animal and measuring protective or therapeutic effect in a second subject.

As used herein, an “immunogenic agent” or “immunogen” is capable of inducing an immunological response against itself on administration to a mammal, optionally in conjunction with an adjuvant.

As used herein, the terms “individual,” “host,” “subject, and “patient” are used interchangeably herein, and refer to a mammal, including, but not limited to, humans, rodents, such as mice and rats, and other laboratory animals.

As used herein, the term “polypeptide” refers to a chain of amino acids of any length, regardless of modification (e.g., phosphorylation or glycosylation). The term polypeptide includes proteins and fragments thereof. The polypeptides can be “exogenous,” meaning that they are “heterologous,” i.e., foreign to the host cell being utilized, such as human polypeptide produced by a bacterial cell. Polypeptides are disclosed herein as amino acid residue sequences. Those sequences are written left to right in the direction from the amino to the carboxy terminus. In accordance with standard nomenclature, amino acid residue sequences are denominated by either a three letter or a single letter code as indicated as follows: Alanine (Ala, A), Arginine (Arg, Asparagine (Asn, N), Aspartic Acid (Asp, D), Cysteine (Cys, C), Glutamine (Gln, Q), Glutamic Acid (Glu, E), Glycine (Gly, G), Histidine (His, H), Isoleucine (Ile, I), Leucine (Leu, L), Lysine (Lys, K), Methionine (Met, M), Phenylalanine (Phe, F), Proline (Pro, P), Serine (Ser, S), Threonine (Thr, T), Tryptophan (Trp, W), Tyrosine (Tyr, Y), and Valine (Val, V).

As used herein, the term “variant” refers to a polypeptide or polynucleotide that differs from a reference polypeptide or polynucleotide, but retains essential properties. A typical variant of a polypeptide differs in amino acid sequence from another, reference polypeptide. Generally, differences are limited so that the sequences of the reference polypeptide and the variant are closely similar overall and, in many regions, identical. A variant and reference polypeptide may differ in amino acid sequence by one or more modifications (e.g., substitutions, additions, and/or deletions). A substituted or inserted amino acid residue may or may not be one encoded by the genetic code. A variant of a polypeptide may be naturally occurring such as an allelic variant, or it may be a variant that is not known to occur naturally.

Modifications and changes can be made in the structure of the polypeptides of the disclosure and still obtain a molecule having similar characteristics as the polypeptide (e.g., a conservative amino acid substitution). For example, certain amino acids can be substituted for other amino acids in a sequence without appreciable loss of activity. Because it is the interactive capacity and nature of a polypeptide that defines that polypeptide's biological functional activity, certain amino acid sequence substitutions can be made in a polypeptide sequence and nevertheless obtain a polypeptide with like properties.

In making such changes, the hydropathic index of amino acids can be considered. The importance of the hydropathic amino acid index in conferring interactive biologic function on a polypeptide is generally understood in the art. It is known that certain amino acids can be substituted for other amino acids having a similar hydropathic index or score and still result in a polypeptide with similar biological activity. Each amino acid has been assigned a hydropathic index on the basis of its hydrophobicity and charge characteristics. Those indices are: isoleucine (+4.5); valine (+4.2); leucine (+3.8); phenylalanine (+2.8); cysteine/cystine (+2.5); methionine (+1.9); alanine (+1.8); glycine (−0.4); threonine (−0.7); serine (−0.8); tryptophan (−0.9); tyrosine (−1.3); proline (−1.6); histidine (−3.2); glutamate (−3.5); glutamine (−3.5); aspartate (−3.5); asparagine (−3.5); lysine (−3.9); and arginine (−4.5).

It is believed that the relative hydropathic character of the amino acid determines the secondary structure of the resultant polypeptide, which in turn defines the interaction of the polypeptide with other molecules, such as enzymes, substrates, receptors, antibodies, antigens, and cofactors. It is known in the art that an amino acid can be substituted by another amino acid having a similar hydropathic index and still obtain a functionally equivalent polypeptide. In such changes, the substitution of amino acids whose hydropathic indices are within ±2 is preferred, those within ±1 are particularly preferred, and those within ±0.5 are even more particularly preferred.

Substitution of like amino acids can also be made on the basis of hydrophilicity, particularly where the biological functional equivalent polypeptide or peptide thereby created is intended for use in immunological embodiments. The following hydrophilicity values have been assigned to amino acid residues: arginine (+3.0); lysine (+3.0); aspartate (+3.0±1); glutamate (+3.0±1); serine (+0.3); asparagine (+0.2); glutamine (+0.2); glycine (0); proline (−0.5±1); threonine (−0.4); alanine (−0.5); histidine (−0.5); cysteine (−1.0); methionine (−1.3); valine (−1.5); leucine (−1.8); isoleucine (−1.8); tyrosine (−2.3); phenylalanine (−2.5); tryptophan (−3.4). It is understood that an amino acid can be substituted for another having a similar hydrophilicity value and still obtain a biologically equivalent, and in particular, an immunologically equivalent polypeptide. In such changes, the substitution of amino acids whose hydrophilicity values are within ±2 is preferred, those within ±1 are particularly preferred, and those within ±0.5 are even more particularly preferred.

As outlined above, amino acid substitutions are generally based on the relative similarity of the amino acid side-chain substituents, for example, their hydrophobicity, hydrophilicity, charge, size, and the like. Exemplary substitutions that take various foregoing characteristics into consideration are well known to those of skill in the art and include (original residue: exemplary substitution): (Ala: Gly, Ser), (Arg: Lys), (Asn: Gln, His), (Asp: Glu, Cys, Ser), (Gln: Asn), (Glu: Asp), (Gly: Ala), (His: Asn, Gln), (Ile: Leu, Val), (Leu: Ile, Val), (Lys: Arg), (Met: Leu, Tyr), (Ser: Thr), (Thr: Ser), (Trp: Tyr), (Tyr: Trp, Phe), and (Val: Ile, Leu). Embodiments of this disclosure thus contemplate functional or biological equivalents of a polypeptide as set forth above. In particular, embodiments of the polypeptides can include variants having about 50%, 60%, 70%, 80%, 90%, 95%, 96%, 97%, 98%, 99%, or more sequence identity to the polypeptide of interest.

The term “percent (%) sequence identity” is defined as the percentage of nucleotides or amino acids in a candidate sequence that are identical with the nucleotides or amino acids in a reference nucleic acid sequence, after aligning the sequences and introducing gaps, if necessary, to achieve the maximum percent sequence identity. Alignment for purposes of determining percent sequence identity can be achieved in various ways that are within the skill in the art, for instance, using publicly available computer software such as BLAST, BLAST-2, ALIGN, ALIGN-2 or Megalign (DNASTAR) software. Appropriate parameters for measuring alignment, including any algorithms needed to achieve maximal alignment over the full-length of the sequences being compared can be determined by known methods.

For purposes herein, the % sequence identity of a given nucleotides or amino acids sequence C to, with, or against a given nucleic acid sequence D (which can alternatively be phrased as a given sequence C that has or comprises a certain % sequence identity to, with, or against a given sequence D) is calculated as follows:

100 times the fraction W/Z,

where W is the number of nucleotides or amino acids scored as identical matches by the sequence alignment program in that program's alignment of C and D, and where Z is the total number of nucleotides or amino acids in D. It will be appreciated that where the length of sequence C is not equal to the length of sequence D, the % sequence identity of C to D will not equal the % sequence identity of D to C.

As used herein, the term “pharmaceutically acceptable carrier” encompasses any of the standard pharmaceutical carriers, such as a phosphate buffered saline solution, water and emulsions such as an oil/water or water/oil emulsion, and various types of wetting agents.

As used herein, the terms “antigenic determinant” and “epitope” are used interchangeably and refer to the structure recognized by an antibody.

As used herein, a “conformational epitope” is an epitope that includes discontinuous sections of the antigen's amino acid sequence. Antibodies bind a conformational epitope based on 3-D surface features, shape, or tertiary structure of the antigen.

As used herein, a “linear epitope” is an epitope that formed by a continuous sequence of amino acids from the antigen. Linear epitopes typically include about 5 to about 10 continuous amino acid residues. Antibodies bind a linear epitope based on the primary sequence of the antigen.

As used herein, a “paratope,” also called an “antigen-binding site,” is a part of an antibody which recognizes and binds to an antigen.

### II. Compositions

A. KLRG1

1. KLRG1 Signaling

Killer cell lectin-like receptor G1 (KLRG1) is an inhibitory receptor expressed on subsets of NK cells and T cells, and interacts primarily with E-cadherin, but also N- and R-cadherins as well. KLRG1 was originally termed mast cell function-associated antigen (MAFA). KLRG1 possesses a cytoplasmic ITIM domain, and experimental evidence indicated that KLRG1 interaction with E-cadherin inhibits NK and T cell function.

KLRG1 is upregulated on NK cells and T cells during acute infections, similar to several well described inhibitory receptors, such as PD-1, and was thought to mark short-lived effector CD8⁺ T cells. However, like PD-1, specific T cell subsets maintain or re-express KLRG1 following initial activation. Additionally, KLRG1 can be upregulated on T cells following stimulation with agonist 4-1BB mAbs, that have cytotoxic activity against melanoma. Therefore, KLRG1 has been used as a marker of activated effector T cells, senescent T cells, terminally-differentiated T cells, and exhausted T cells.

KLRG1 is clearly expressed after T cell activation and may remain expressed on both effector and central memory T cells. In support of the notion of expression on both memory T cells and senescent T cells, the percentage of T cells expressing KLRG1 in humans increases with age, where up 90% of CD8⁺ T cells may express KLRG1 in individuals over 65 years of age. KLRG1 is also predominantly found on the most mature CD56^{(dim)} NK cells in humans. Finally, studies have also shown that KLRG1 is associated with dysfunctional, or exhausted, T cells and is co-expressed with the exhaustion markers PD-1, 2B4, CD160 and LAG-3 and others. Overall, a clear pattern emerges that while KLRG1 is upregulated during the T cell effector phase, its major role is to modulate the T cell response, similar to other well described inhibitory receptors that are expressed following T cell activation.

An inhibitory function for KLRG1 has been described for both NK cells and T cells. Expression on NK cells negatively correlate with effector function. Additionally, NK cells were more easily activated in the presence of cells with E-cadherin mutations. In the case of T cells, blockade of KLRG1 interaction with E-cadherin also resulted in significantly increased responses in human T cells. Together, these data show that KLRG1 is capable of inhibiting both NK and effector T cells.

Moreover, a recent report demonstrated biological function of an anti-hKLRG1 blocking antibody using PBMC from HCV patients. Coinfection of hepatitis B virus (HBV) with hepatitis C virus (HCV) is quite common, leading to an increase in morbidity and mortality. As such, HBV vaccination is recommended in HCV-infected individuals. However, HBV vaccine responses in HCV-infected individuals are often blunted compared with uninfected populations. The mechanism for this failure of vaccine response in HCV-infected subjects remains unclear. In Shi et al. (2014), they investigated function of KLRG1 in the regulation of CD4+ T cells and HBV vaccine responses during HCV infection. KLRG1 was overexpressed on CD4+ T cells from HCV-infected, HBV vaccine non-responders compared with HBV vaccine responders. The capacity of CD4+ T cells to proliferate and secrete IL-2 cytokine was inversely associated with the level of KLRG1 expression. Importantly, blocking KLRG1 signaling resulted in a significant improvement in CD4+ T cell proliferation and IL-2 production in HCV-infected, HBV vaccine non-responders in response to TCR stimulation. This study clearly demonstrated co-inhibitory function of KLRG1 in a chronic infection scenario.

Others have shown that KLRG1 is highly expressed on CD4⁺ FoxP3⁺ regulatory T cells (Tregs). Research in ovarian cancer models have indicated that KLRG1 expression is largely restricted to CD4⁺FoxP3⁺ Tregs, and that KLRG1⁺ Tregs accumulate in mouse tumor ascites (Flies, unpublished). However, the expression and function on human Treg cells has not been clearly elucidated.

While a similar pattern of expression is observed in mice, because of the vast difference in lifespan between human and mouse, it may be difficult to truly assess the function of KLRG1 in murine models. As such, studies in KLRG1 KO mice have indicated no change in immune phenotype or functional response against viral infections, although detailed study of KLRG1 KO mice remains rather lacking at this time. Nevertheless, studies have indicated the KLRG1-E-cadherin interaction induce bidirectional signaling, and signaling through E-cadherin⁺ DCs influences DC adhesion and function. Moreover, signaling through E-cadherin-Beta-catenin pathways may promote tolerogenic DCs that have negative effects on anti-tumor immunity. Therefore, disruption of KLRG1-E-cadherin interactions may eliminate bidirectional negative regulatory signaling pathways.

Although structural and ligand-binding properties of human (h) and mouse (m) KLRG1 are very similar, a recent report from Hanspeter Pircher's group showed structure difference between hKLRG1 and mKLRG1. Biochemical analyses further showed that mKLRG1 formed monomers and disulfide-linked dimers, trimers, and tetramers whereas hKLRG1 was exclusively present as disulfide-linked dimer. These data indicate hKLRG1 and mKLRG1 have different structural and biological functions and the activity of one may not be predictive of the other.

2. KLRG1 Sequences

The protein sequence of the extracellular domain (ECD) for human KLRG1 is LCQGSNYSTCASCPSCPDRWMKYGNHCYYFSVEEKDWNSSLEFCLARDSHLLVITD NQEMSLLQVFLSEAFCWIGLRNNSGWRWEDGSPLNFSRISSNSFVQTCGAINKNGLQ ASSCEVPLHWVCKKCPFADQALF (SEQ ID NO:1).

The protein sequence of the ECD for mouse KLRG1 is QRILCCGSKDSTCSHCPSCPILWTRNGSHCYYFSMEKKDWNSSLKFCADKGSHLLTF PDNQGVKLFGEYLGQDFYWIGLRNIDGWRWEGGPALSLRILTNSLIQRCGAIHRNGL QASSCEVALQWICKKVLY (SEQ ID NO:2).

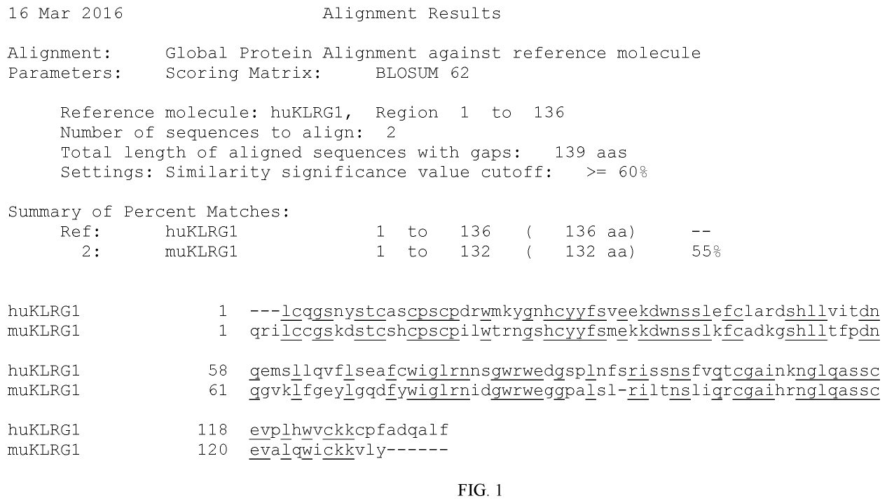

FIG. 1 shows the sequence alignment of human and mouse KLRG1 extracellular domains.

The amino acid sequence for the KLRG1 Endo-domain is MTDSVIYSMLELPTATQAQNDYGPQQKSSSSRPSCSCL (SEQ ID NO:3). The underlined portion shows a typical ITIM domain.

3. KLRG1 Ligands

KLRG1 binds to E-, N- and R-Cadherins. Cadherins are a large family of transmembrane glycoproteins that largely engage in homotypic interactions, mediate calcium dependent cell-cell adhesion and are a major constituent of adherens junctions in epithelial cells. E-cadherin is also expressed on subsets of DCs, where it may promote a context dependent tolerogenic or inflammatory phenotype and function. E-cadherin also binds the integrin CD103 (ITGAE, aEb7), which is expressed by DC subsets, CD8+ T cell subsets and Treg cell subsets.

B. KLRG1 Binding Molecules

One embodiment provides KLRG1-binding molecules including but not limited to antibodies and fusion proteins. In one embodiment, the antibodies and fusion proteins block signal transduction through KLRG1. In another embodiment, the antibodies and fusion proteins induce or enhance signal transduction through KLRG1.

The antibodies and fusion proteins can block the interaction between KLRG1 and its ligands including, but not limited to E-cadherin. In one embodiment, the antibodies and fusion proteins bind the ECD of KLRG1 without triggering signal transduction through KLRG1. Such binding can prevent ligands of KLRG1 from binding KLRG1. In another embodiment, the antibodies and fusion proteins crosslink one or more KLRG1 extracellular domains and thereby inhibit or reduce the interaction of KLRG1 and its ligands such as E-, N- and R-Cadherins.

In another embodiment the antibodies and fusion proteins can induce or enhance the interaction between KLRG1 and its ligands including, but not limited to E-, N- and R-Cadherins. In still another embodiment, the antibodies and fusion proteins bind the ECD of KLRG1 and trigger signal transduction through KLRG1.

For example, the disclosed molecules can immunospecifically bind to KLRG1 (e.g., SEQ ID NO:1, SEQ ID NO:2, etc.). For example, molecules are provided that can immunospecifically bind to human KLRG1:

(I) arrayed on the surface of a cell (preferably a live cell);

(II) arrayed on the surface of a cell (preferably a live cell) at an endogenous concentration;

(III) arrayed on the surface of a live cell, and modulates binding between KLRG1 and a ligand thereof;

(IV) arrayed on the surface of a live cell, and reduces, prevents, or inhibits TGF-β secretion;

(V) arrayed on the surface of a live cell, wherein the cell is a myeloid cell such as a macrophage or dendritic cell, or a cancer cell (e.g., brain cancer cell, renal cell carcinoma cell (RCC), Ewing sarcoma cell, breast cancer cell, or ovarian cancer cell);

(VI) combinations thereof.

KLRG1 binding molecules, including antibodies and antigen binding fragments thereof, and fusion proteins that bind to one or more KLRG1 polypeptides or fusion proteins, or fragments or variants thereof are disclosed. The antibodies disclosed herein are typically monoclonal antibodies, or antigen binding fragments thereof, that bind to an epitope present on a KLRG1 polypeptide, or fragment or fusion thereof. In some embodiments the antibody binds to a conformational epitope. In some embodiments the antibody binds to a linear epitope. A linear epitope can be 4, 5, 6, 7, 8, 9, 10, 11, or more continuous amino acids in length. The epitope can include one or more non-amino acid elements, post-translation modifications, or a combination thereof. Examples of post-translational modifications include, but are not limited to glycosylation, phosphorylation, acetylation, citrullination and ubiquitination. For example, antibodies can bind an epitope that is formed at least in-part by one or more sugar groups.

The antibody or antigen binding fragment thereof can bind to an epitope that is present on an endogenous KLRG1 polypeptide, or a recombinant KLRG1 polypeptide, or a combination thereof. In some embodiments, the antibody or antigen binding fragment thereof binds to the extracellular domain, or a fragment thereof, or an epitope formed therefrom of KLRG1. In some embodiments, the antibody or antigen binding fragment thereof is a function blocking antibody that reduces or prevents KLRG1 from binding to one or more of its ligands, reduces intracellular signaling modulated by KLRG1 or a combination thereof. In some embodiments, the antibody or antigen binding fragment thereof is a function activating antibody that induces, promotes, or enhances KLRG1 to bind to one or more of its ligands, and stimulates, enhances, or promotes KLRG1-mediated signal transduction.

1. Anti-KLRG1 Antibody Sequences

a. K1A6 Sequences

In one embodiment a murine monoclonal antibody is produced by hybridoma clone K1A6 and consists of two light chains and two heavy chains.

i. Light Chain

One embodiment provides a murine monoclonal antibody or antigen binding fragment thereof that has a light chain having at least 50%, 60%, 70%, 80%, 85%, 90%, 95%, 99%, or 100% sequence identity with the following amino acid sequence:

(SEQ ID NO: 4)
DIVMTQSHKFMSTSVGDRVSITCKASQDVATAVDWYQQKPGQSPKLLIYWA
STRHTGVPDRFTGSGSGTDFTLTISNVQSEDLADYFCQQYSSYPLTFGTGT
KLELK.

The CDRs are of SEQ ID NO:4 are bolded and underlined and are:

CDR1
(SEQ ID NO: 5)
KASQDVATAVD;
CDR2
(SEQ ID NO: 6)
WASTRHT;
and
CDR3
(SEQ ID NO: 7)
QQYSSYPLT.

Another embodiment provides a nucleic acid that encodes the light chain (SEQ ID NO:4).

An exemplary nucleic acid that encodes light chain (SEQ ID NO:4) is GACATTGTGATGACCCAGTCTCACAAATTCATGTCCACATCAGTAGGAGACAGG GTCAGCATCACCTGCAAGGCCAGTCAGGATGTGGCTACTGCTGTAGACTGGTATC AACAGAAACCAGGGCAATCTCCTAAACTACTGATTTATTGGGCATCTACCCGGCA CACTGGAGTCCCTGATCGCTTCACAGGCAGTGGATCTGGGACAGATTTCACTCTC ACCATTAGCAATGTGCAGTCTGAAGACTTGGCAGATTATTTCTGTCAGCAATATA GCAGCTATCCTCTCACGTTCGGTACTGGGACCAAGCTGGAGCTGAAA (SEQ ID NO:8).

ii. Heavy Chain

One embodiment provides a murine monoclonal antibody or antigen binding fragment thereof that has a heavy chain having at least 50%, 60%, 70%, 80%, 85%, 90%, 95%, 99%, or 100% sequence identity with the following amino acid sequence:

(SEQ ID NO: 9)
EIQLQQSGAELVKPGASVKISCKASGYSFTGYNMNWVRQSHGKSLEWIGNI
NPYFGSSIYNQRFKGKATLTVDKSSSTAYMQLNSLTSEDSAVYYCAKNYDY
ERDAMDKWGQGTSVTVSS.

The CDRs are of SEQ ID NO:9 are bolded and underlined and are:

CDR1
(SEQ ID NO: 10)
GYNMN;
CDR2
(SEQ ID NO: 11)
NINPYFGSSIYNQRFKG;
and
CDR3
(SEQ ID NO: 12)
NYDYERDAMDK.

Another embodiment provides a nucleic acid encoding heavy chain (SEQ ID NO:9). An exemplary nucleic acid that encodes heavy chain (SEQ ID NO:9) is GAGATCCAGCTGCAGCAGTCTGGAGCTGAGCTGGTGAAGCCTGGGGCTTCAGTG AAGATATCCTGCAAGGCTTCTGGTTACTCATTCACTGGCTACAACATGAACTGGG TGAGGCAGAGCCATGGAAAGAGCCTTGAGTGGATTGGAAATATTAATCCTTACTT TGGTAGTTCTATCTACAATCAGAGGTTTAAGGGCAAGGCCACATTGACTGTAGAC AAATCCTCCAGCACAGCCTACATGCAGCTCAACAGCCTGACATCTGAGGACTCA GCAGTCTATTACTGTGCGAAAAACTATGATTACGAGAGAGATGCTATGGACAAA TGGGGTCAAGGAACCTCAGTCACCGTCTCCTCA (SEQ ID NO:13).

One embodiment provides an antibody, preferably a monoclonal antibody, or antigen binding fragment thereof that has a light chain containing CDRs according to SEQ ID Nos: 5, 6, and 7.

One embodiment provides an antibody, preferably a monoclonal antibody, or an antigen binding fragment thereof having a heavy chain containing CDRs according to SEQ ID Nos: 10, 11, and 12.

One embodiment provides an antibody, preferably a monoclonal antibody, or antigen binding fragment thereof that has a light chain containing CDRs according to SEQ ID Nos: 5, 6, and 7 and a heavy chain containing CDRs according to SEQ ID Nos: 10, 11, and 12.

One embodiment provides an antibody, preferably a monoclonal antibody, or an antigen binding fragment thereof having a light chain at least 50%, 60%, 70%, 80%, 85%, 90%, 95%, 99%, or 100% sequence identity to SEQ ID NO:4 and a heavy chain having at least 50%, 60%, 70%, 80%, 85%, 90%, 95%, 99%, or 100% sequence identity to SEQ ID NO:9.

b. K1B9

In one embodiment a murine monoclonal antibody is produced by hybridoma clone K1B9 and consists of two light chains and two heavy chains.

i. Light Chain

One embodiment provides a murine monoclonal antibody or antigen binding fragment thereof that has a light chain having at least 50%, 60%, 70%, 80%, 85%, 90%, 95%, 99%, or 100% sequence identity with the following amino acid sequence:

(SEQ ID NO: 14)
DIVLTQSPATLSVTLGDSVSLSCRASQSISNNLHWYQQKSHESPRLLIKYA
SQSISGIPSRFSGSGSGTDFTLSINSVETEDFGMYFCQQSNSWPHTFGGGT
KLEIK.

The CDRs are of SEQ ID NO:14 are bolded and underlined and are:

CDR1
(SEQ ID NO: 15)
RASQSISNNLH;
CDR2
(SEQ ID NO: 16)
YASQSIS;
and
CDR3
(SEQ ID NO: 17)
QQSNSWPHT.

Another embodiment provides a nucleic acid that encodes the light chain (SEQ ID NO:14).

An exemplary nucleic acid that encodes light chain (SEQ ID NO:14) is GATATTGTGCTAACTCAGTCTCCAGCCACCCTGTCTGTGACTCTAGGAGATAGCG TCAGTCTTTCCTGCAGGGCCAGCCAAAGTATTAGCAACAACCTACACTGGTATCA ACAAAAATCACATGAGTCTCCAAGGCTTCTCATTAAGTATGCTTCCCAGTCCATC TCTGGGATCCCCTCCAGGTTCAGTGGCAGTGGATCAGGGACAGATTTCACTCTCA GTATCAACAGTGTGGAGACTGAAGATTTTGGAATGTATTTCTGTCAACAGAGTAA CAGCTGGCCTCATACGTTCGGAGGGGGGACCAAGCTGGAAATAAAA (SEQ ID NO:18).

ii. Heavy Chain

One embodiment provides a murine monoclonal antibody or antigen binding fragment thereof that has a heavy chain having at least 50%, 60%, 70%, 80%, 85%, 90%, 95%, 99%, or 100% sequence identity with the following amino acid sequence:

(SEQ ID NO: 19)
EVQLQQSGPELVKPGASVKMSCKASGYTFTGYVMHWVKQKPGQGLEWIGYI
NPYNDGTRYSEKFKGKATLTSDKSSSTAYMELSSLTSEDSAVYYCARGRDY
WGQGTTLTVSS.

The CDRs are of SEQ ID NO:19 are bolded and underlined and are:

CDR1
(SEQ ID NO: 20)
GYVMH;
CDR2
(SEQ ID NO: 21)
YINPYNDGTRYSEKFKG;
and
CDR3
(SEQ ID NO: 22)
GRDY.

Another embodiment provides a nucleic acid encoding heavy chain (SEQ ID NO:19). An exemplary nucleic acid that encodes heavy chain (SEQ ID NO:19) is GAGGTCCAGCTGCAGCAGTCTGGACCTGAACTGGTAAAGCCTGGGGCTTCAGTG AAGATGTCCTGCAAGGCTTCTGGATACACATTCACTGGCTATGTTATGCACTGGG TGAAGCAGAAGCCTGGGCAGGGCCTTGAGTGGATTGGATATATTAATCCTTACA ATGATGGTACTAGGTACAGTGAGAAGTTCAAAGGCAAGGCCACACTGACTTCAG ACAAATCCTCCAGCACAGCCTACATGGAGCTCAGCAGCCTGACCTCTGAGGACT CTGCGGTCTATTACTGTGCAAGAGGGCGGGACTACTGGGGCCAAGGCACCACTC TCACAGTCTCCTCA (SEQ ID NO:23).

One embodiment provides an antibody, preferably a monoclonal antibody, or antigen binding fragment thereof that has a light chain containing CDRs according to SEQ ID Nos: 15, 16, and 17.

One embodiment provides an antibody, preferably a monoclonal antibody, or an antigen binding fragment thereof having a heavy chain containing CDRs according to SEQ ID Nos: 20, 21, and 22.

One embodiment provides an antibody, preferably a monoclonal antibody, or antigen binding fragment thereof that has a light chain containing CDRs according to SEQ ID Nos: 15, 16, and 17 and a heavy chain containing CDRs according to SEQ ID Nos: 20, 21, and 22.

One embodiment provides an antibody, preferably a monoclonal antibody, or an antigen binding fragment thereof having a light chain at least 50%, 60%, 70%, 80%, 85%, 90%, 95%, 99%, or 100% sequence identity to SEQ ID NO:14 and a heavy chain having at least 50%, 60%, 70%, 80%, 85%, 90%, 95%, 99%, or 100% sequence identity to SEQ ID NO:19.

c. K1F7

In one embodiment a murine monoclonal antibody is produced by hybridoma clone K1F7 and consists of two light chains and two heavy chains.

i. Light Chain

One embodiment provides a murine monoclonal antibody or antigen binding fragment thereof that has a light chain having at least 50%, 60%, 70%, 80%, 85%, 90%, 95%, 99%, or 100% sequence identity with the following amino acid sequence:

(SEQ ID NO: 24)
DIVMTQSQKFMSTSVGDRVSITCKASQNVGSTVVWYQQKPGQSPKLLIYSA
SNRYTGVPDRFTGNGSGTDFTLTISNMQSEDLADYFCQQCSSYPLTFGAGT
KLELK.

The CDRs are of SEQ ID NO:24 are bolded and underlined and are:

CDR1
(SEQ ID NO: 25)
KASQNVGSTVV;
CDR2
(SEQ ID NO: 26)
SASNRYT;
and
CDR3
(SEQ ID NO: 27)
QQCSSYPLT.

Another embodiment provides a nucleic acid that encodes the light chain (SEQ ID NO:24).

An exemplary nucleic acid that encodes light chain (SEQ ID NO:24) is GACATTGTGATGACCCAGTCTCAAAAATTCATGTCCACATCAGTAGGAGACAGG GTCAGCATCACCTGCAAGGCCAGTCAGAATGTGGGTAGTACTGTAGTCTGGTATC AACAGAAACCAGGACAATCTCCTAAACTACTGATTTACTCGGCATCCAATCGGTA CACTGGAGTCCCTGATCGCTTCACAGGCAATGGATCTGGGACAGATTTCACTCTC ACCATCAGCAATATGCAGTCTGAAGACCTGGCAGATTATTTCTGCCAGCAATGTA GCAGCTATCCTCTCACGTTCGGTGCTGGGACCAAGCTGGAGCTGAAA (SEQ ID NO:28).

ii. Heavy Chain

One embodiment provides a murine monoclonal antibody or antigen binding fragment thereof that has a heavy chain having at least 50%, 60%, 70%, 80%, 85%, 90%, 95%, 99%, or 100% sequence identity with the following amino acid sequence:

(SEQ ID NO: 29)
QVQLQQSGAELVRPGASVKISCKAFGYTFTNHFINWVKQRPGQGLDWIGYI
NPYNGYTNYNQKFKGKATLTVDKSSSTACMELSSLTSEDSAVYYCAISYDG
YYERFAYWGQGTLVTVSA.

The CDRs are of SEQ ID NO:29 are bolded and underlined and are:

CDR1
(SEQ ID NO: 30)
NHFIN;
CDR2
(SEQ ID NO: 31)
YINPYNGYTNYNQKFKG;
and
CDR3
(SEQ ID NO: 32)
SYDGYYERFAY.

Another embodiment provides a nucleic acid encoding heavy chain (SEQ ID NO:29).

An exemplary nucleic acid that encodes heavy chain (SEQ ID NO:29) is CAGGTCCAGCTGCAGCAGTCTGGGGCTGAGCTGGTGAGGCCTGGGGCCTCAGTG AAGATTTCCTGCAAGGCTTTTGGCTACACCTTCACAAACCATTTTATAAACTGGG TGAAGCAGAGGCCTGGACAGGGCCTGGACTGGATTGGATATATTAATCCTTATA ATGGTTATACAAACTACAACCAGAAGTTCAAGGGCAAGGCCACATTGACTGTAG ACAAATCCTCCAGCACAGCCTGTATGGAGCTTAGCAGCCTGACATCTGAGGACTC TGCAGTCTATTACTGTGCCATATCCTATGATGGTTACTACGAGAGGTTTGCTTACT GGGGCCAAGGGACTCTGGTCACTGTCTCTGCA (SEQ ID NO:33).

One embodiment provides an antibody, preferably a monoclonal antibody, or antigen binding fragment thereof that has a light chain containing CDRs according to SEQ ID Nos: 25, 26, and 27.

One embodiment provides an antibody, preferably a monoclonal antibody, or an antigen binding fragment thereof having a heavy chain containing CDRs according to SEQ ID Nos: 30, 31, and 32.

One embodiment provides an antibody, preferably a monoclonal antibody, or antigen binding fragment thereof that has a light chain containing CDRs according to SEQ ID Nos: 25, 26, and 27 and a heavy chain containing CDRs according to SEQ ID Nos: 30, 31, and 32.

One embodiment provides an antibody, preferably a monoclonal antibody, or an antigen binding fragment thereof having a light chain at least 50%, 60%, 70%, 80%, 85%, 90%, 95%, 99%, or 100% sequence identity to SEQ ID NO:24 and a heavy chain having at least 50%, 60%, 70%, 80%, 85%, 90%, 95%, 99%, or 100% sequence identity to SEQ ID NO:29.

d. K2H11¶

In one embodiment a murine monoclonal antibody is produced by hybridoma clone K2H11 and consists of two light chains and two heavy chains.

i. Light Chain

One embodiment provides a murine monoclonal antibody or antigen binding fragment thereof that has a light chain having at least 50%, 60%, 70%, 80%, 85%, 90%, 95%, 99%, or 100% sequence identity with the following amino acid sequence:

(SEQ ID NO: 34)
ENVLTQSPAIMSASPGEKVTMTCRASSSVSSNYLHWYQQKSGASPKIWIYS
TSNLASAVPARFSGSGSGTSYSLTISSVEAEDAATYYCQQYSGYPLTFGGG
TKLEIK.

The CDRs are of SEQ ID NO:34 are bolded and underlined and are:

CDR1
(SEQ ID NO: 35)
RASSSVSSNYLH;
CDR2
(SEQ ID NO: 36)
STSNLAS;
and
CDR3
(SEQ ID NO: 37)
QQYSGYPLT.

Another embodiment provides a nucleic acid that encodes the light chain (SEQ ID NO:34).

An exemplary nucleic acid that encodes light chain (SEQ ID NO:34) is GAAAATGTGCTCACCCAGTCTCCAGCAATCATGTCTGCATCTCCAGGGGAAAAG GTCACCATGACCTGCAGGGCCAGCTCAAGTGTTAGTTCCAATTACTTGCACTGGT ACCAGCAGAAGTCAGGTGCCTCCCCCAAAATCTGGATTTATAGCACATCCAATCT GGCTTCTGCAGTCCCTGCGCGCTTCAGTGGCAGTGGGTCTGGGACCTCTTACTCT CTCACAATCAGCAGTGTGGAGGCTGAAGATGCTGCCACTTATTACTGCCAGCAGT ACAGTGGTTACCCACTGACGTTCGGTGGAGGCACCAAGTTGGAAATCAAA (SEQ ID NO:38).

ii. Heavy Chain

One embodiment provides a murine monoclonal antibody or antigen binding fragment thereof that has a heavy chain having at least 50%, 60%, 70%, 80%, 85%, 90%, 95%, 99%, or 100% sequence identity with the following amino acid sequence:

(SEQ ID NO: 39)
QVQLQQPGAEFVKPGASVKLSCKASGYTFTSYWIHWMKQRPGQGLEWIGNI
YPGRSNNNYNEKFKNRATLTVDTSSSTAYMQFRSLTSDDSAVYYCARDATV
EPLPYWGQGTLVTVSA.

The CDRs are of SEQ ID NO:39 are bolded and underlined and are:

CDR1
(SEQ ID NO: 40)
SYWIH;
CDR2
(SEQ ID NO: 41)
NIYPGRSNNNYNEKFKN;
and
CDR3
(SEQ ID NO: 42)
DATVEPLPY.

Another embodiment provides a nucleic acid encoding heavy chain (SEQ ID NO:39). An exemplary nucleic acid that encodes heavy chain (SEQ ID NO:39) is CAGGTCCAACTGCAGCAGCCTGGTGCTGAGTTTGTGAAGCCTGGGGCCTCAGTG AAGCTGTCGTGCAAGGCTTCTGGCTACACTTTCACCAGCTACTGGATACATTGGA TGAAGCAGAGGCCTGGACAAGGCCTTGAGTGGATTGGAAATATTTATCCTGGTA GAAGTAATAATAACTACAATGAGAAGTTCAAGAACAGGGCCACACTGACTGTAG ACACATCCTCCAGCACAGCCTACATGCAGTTCAGAAGCCTGACATCTGACGACTC TGCGGTCTATTATTGTGCAAGAGATGCTACGGTGGAGCCTCTTCCTTACTGGGGC CAAGGGACTCTGGTCACTGTCTCTGCA (SEQ ID NO:43).

One embodiment provides an antibody, preferably a monoclonal antibody, or antigen binding fragment thereof that has a light chain containing CDRs according to SEQ ID Nos: 35, 36, and 37.

One embodiment provides an antibody, preferably a monoclonal antibody, or an antigen binding fragment thereof having a heavy chain containing CDRs according to SEQ ID Nos: 40, 41, and 42.

One embodiment provides an antibody, preferably a monoclonal antibody, or antigen binding fragment thereof that has a light chain containing CDRs according to SEQ ID Nos: 35, 36, and 37 and a heavy chain containing CDRs according to SEQ ID Nos: 40, 41, and 42.

One embodiment provides an antibody, preferably a monoclonal antibody, or an antigen binding fragment thereof having a light chain at least 50%, 60%, 70%, 80%, 85%, 90%, 95%, 99%, or 100% sequence identity to SEQ ID NO:34 and a heavy chain having at least 50%, 60%, 70%, 80%, 85%, 90%, 95%, 99%, or 100% sequence identity to SEQ ID NO:39.

e. K3G4

In one embodiment a murine monoclonal antibody is produced by hybridoma clone K3G4 and consists of two light chains and two heavy chains.

i. Light Chain

One embodiment provides a murine monoclonal antibody or antigen binding fragment thereof that has a light chain having at least 50%, 60%, 70%, 80%, 85%, 90%, 95%, 99%, or 100% sequence identity with the following amino acid sequence:

(SEQ ID NO: 44)
EIVLTQSPALMAASPGEKVTITCSVSSSISSSNLHWYQQKSETSPKPWIYG
TSNLASGVPVRFSGSGSGTSYSLTISSMEAEDAATYYCQQWSSYPLTFGAG
TKLELK.

The CDRs are of SEQ ID NO:44 are bolded and underlined and are:

CDR1
(SEQ ID NO: 45)
SVSSSISSSNLH;
CDR2
(SEQ ID NO: 46)
GTSNLAS;
and
CDR3
(SEQ ID NO: 47)
QQWSSYPLT.

Another embodiment provides a nucleic acid that encodes the light chain (SEQ ID NO:44).

An exemplary nucleic acid that encodes light chain (SEQ ID NO:44) is GAAATTGTGCTCACCCAGTCTCCAGCACTCATGGCTGCATCTCCAGGGGAGAAG GTCACCATCACCTGCAGTGTCAGCTCAAGTATAAGTTCCAGCAACTTGCACTGGT ACCAGCAGAAGTCAGAAACCTCCCCCAAACCCTGGATTTATGGCACATCCAACC TGGCTTCTGGAGTCCCTGTTCGCTTCAGTGGCAGTGGATCTGGGACCTCTTATTCT CTCACAATCAGCAGCATGGAGGCTGAAGATGCTGCCACTTATTACTGTCAACAGT GGAGTAGTTACCCACTCACGTTCGGTGCTGGGACCAAGCTGGAGCTGAAA (SEQ ID NO:48).

ii. Heavy Chain

One embodiment provides a murine monoclonal antibody or antigen binding fragment thereof that has a heavy chain having at least 50%, 60%, 70%, 80%, 85%, 90%, 95%, 99%, or 100% sequence identity with the following amino acid sequence:

(SEQ ID NO: 49)
QVQLQQPGAELVKPGASVKLSCKASGYTFTSYWINWVKQRPGQGLEWIGNI
YPGSSSTNYNEKFKYKATLTVDTSSSTANMQLSSLTSDDSAVYYCARGRLL
RLRRGGYFDYWGQGTTLTVSS.

The CDRs are of SEQ ID NO:49 are bolded and underlined and are:

CDR1
(SEQ ID NO: 50)
SYWIN;
CDR2
(SEQ ID NO: 51)
NIYPGSSSTNYNEKFKY;
and
CDR3
(SEQ ID NO: 52)
GRLLRLRRGGYFDY.

Another embodiment provides a nucleic acid encoding heavy chain (SEQ ID NO:49). An exemplary nucleic acid that encodes heavy chain (SEQ ID NO:49) is CAGGTCCAACTGCAGCAGCCTGGTGCTGAGCTTGTGAAGCCTGGGGCCTCAGTG AAGCTGTCCTGCAAGGCTTCTGGCTACACTTTCACCAGCTACTGGATAAACTGGG TGAAGCAGAGGCCTGGACAAGGCCTTGAGTGGATTGGAAATATTTATCCTGGTA GTAGTAGTACTAATTACAATGAGAAGTTCAAGTACAAGGCCACACTGACTGTAG ACACATCCTCCAGTACAGCCAACATGCAGCTCAGCAGCCTGACATCTGACGACTC TGCGGTCTATTATTGTGCAAGAGGTCGTTTATTACGGCTAAGACGAGGGGGCTAC TTTGACTACTGGGGCCAAGGCACCACTCTCACAGTCTCCTCA (SEQ ID NO:53).

One embodiment provides an antibody, preferably a monoclonal antibody, or antigen binding fragment thereof that has a light chain containing CDRs according to SEQ ID Nos: 45, 46, and 47.

One embodiment provides an antibody, preferably a monoclonal antibody, or an antigen binding fragment thereof having a heavy chain containing CDRs according to SEQ ID Nos: 50, 51, and 52.

One embodiment provides an antibody, preferably a monoclonal antibody, or antigen binding fragment thereof that has a light chain containing CDRs according to SEQ ID Nos: 45, 46, and 47 and a heavy chain containing CDRs according to SEQ ID Nos: 50, 51, and 52.

One embodiment provides an antibody, preferably a monoclonal antibody, or an antigen binding fragment thereof having a light chain at least 50%, 60%, 70%, 80%, 85%, 90%, 95%, 99%, or 100% sequence identity to SEQ ID NO:44 and a heavy chain having at least 50%, 60%, 70%, 80%, 85%, 90%, 95%, 99%, or 100% sequence identity to SEQ ID NO:49.

f. K3H11

In one embodiment a murine monoclonal antibody is produced by hybridoma clone K3H11 and consists of two light chains and two heavy chains.

i. Light Chain

One embodiment provides a murine monoclonal antibody or antigen binding fragment thereof that has a light chain having at least 50%, 60%, 70%, 80%, 85%, 90%, 95%, 99%, or 100% sequence identity with the following amino acid sequence:

(SEQ ID NO: 24)
DIVMTQSQKFMSTSVGDRVSITCKASQNVGSTVVWYQQKPGQSPKLLIYSA
SNRYTGVPDRFTGNGSGTDFTLTISNMQSEDLADYFCQQCSSYPLTFGAGT
KLELK.

The CDRs are of SEQ ID NO:24 are bolded and underlined and are:

CDR1
(SEQ ID NO: 25)
KASQNVGSTVV;
CDR2
(SEQ ID NO: 26)
SASNRYT;
and
CDR3
(SEQ ID NO: 27)
QQCSSYPLT.

Another embodiment provides a nucleic acid that encodes the light chain (SEQ ID NO:24).

An exemplary nucleic acid that encodes light chain (SEQ ID NO:24) is GACATTGTGATGACCCAGTCTCAAAAATTCATGTCCACATCAGTAGGAGACAGG GTCAGCATCACCTGCAAGGCCAGTCAGAATGTGGGTAGTACTGTAGTCTGGTATC AACAGAAACCAGGACAATCTCCTAAACTACTGATTTACTCGGCATCCAATCGGTA CACTGGAGTCCCTGATCGCTTCACAGGCAATGGATCTGGGACAGATTTCACTCTC ACCATCAGCAATATGCAGTCTGAAGACCTGGCAGATTATTTCTGCCAGCAATGTA GCAGCTATCCTCTCACGTTCGGTGCTGGGACCAAGCTGGAGCTGAAA (SEQ ID NO:54).

ii. Heavy Chain

One embodiment provides a murine monoclonal antibody or antigen binding fragment thereof that has a heavy chain having at least 50%, 60%, 70%, 80%, 85%, 90%, 95%, 99%, or 100% sequence identity with the following amino acid sequence:

(SEQ ID NO: 55)
QVQLQQSGAELVRPGASVKISCKAFGYTFTNHFINWVKQRPGQGLDWIGYI
NPYNGYTNYNQKFKGKATLTVDKSSSTAYMELSSLTSEDSAVYYCAISYDG
YYERFAYWGQGTLVTVSA.

The CDRs are of SEQ ID NO:55 are bolded and underlined and are:

CDR1
(SEQ ID NO: 30)
NHFIN;
CDR2
(SEQ ID NO: 31)
YINPYNGYTNYNQKFKG;
and
CDR3
(SEQ ID NO: 32)
SYDGYYERFAY.

Another embodiment provides a nucleic acid encoding heavy chain (SEQ ID NO:55). An exemplary nucleic acid that encodes heavy chain (SEQ ID NO:55) is CAGGTCCAGCTGCAGCAGTCTGGGGCTGAGCTGGTGAGGCCTGGGGCCTCAGTG AAGATTTCCTGCAAGGCTTTTGGCTACACCTTCACAAACCATTTTATAAACTGGG TGAAGCAGAGGCCTGGACAGGGCCTGGACTGGATTGGATATATTAATCCTTATA ATGGTTATACAAACTACAACCAGAAGTTCAAGGGCAAGGCCACATTGACTGTAG ACAAATCCTCCAGCACAGCCTATATGGAGCTTAGCAGCCTGACATCTGAGGACTC TGCAGTCTATTACTGTGCCATATCCTATGATGGTTACTACGAGAGGTTTGCTTACT GGGGCCAAGGGACTCTGGTCACTGTCTCTGCA (SEQ ID NO:56).

One embodiment provides an antibody, preferably a monoclonal antibody, or antigen binding fragment thereof that has a light chain containing CDRs according to SEQ ID Nos: 25, 26, and 27 and a heavy chain containing CDRs according to SEQ ID Nos: 29, 30, and 32.

One embodiment provides an antibody, preferably a monoclonal antibody, or an antigen binding fragment thereof having a light chain at least 50%, 60%, 70%, 80%, 85%, 90%, 95%, 99%, or 100% sequence identity to SEQ ID NO:24 and a heavy chain having at least 50%, 60%, 70%, 80%, 85%, 90%, 95%, 99%, or 100% sequence identity to SEQ ID NO:55.

g. K4C10

In one embodiment a murine monoclonal antibody is produced by hybridoma clone K4C10 and consists of two light chains and two heavy chains.

i. Light Chain

One embodiment provides a murine monoclonal antibody or antigen binding fragment thereof that has a light chain having at least 50%, 60%, 70%, 80%, 85%, 90%, 95%, 99%, or 100% sequence identity with the following amino acid sequence:

(SEQ ID NO: 57)
DIQMTQSPSSLSASLGERVSLTCRASQDIGSSLNWLQQEPDGTIKRLIYAT
SSLDSGVPKRFSGSRSGSDYSLTISSLESEDFVDYYCLQYASSPPTFGGGT
KLEIK.

The CDRs are of SEQ ID NO:57 are bolded and underlined and are:

CDR1
(SEQ ID NO: 58)
RASQDIGSSLN;
CDR2
(SEQ ID NO: 59)
ATSSLDS;
and
CDR3
(SEQ ID NO: 60)
LQYASSPPT.

Another embodiment provides a nucleic acid that encodes the light chain (SEQ ID NO:57).

An exemplary nucleic acid that encodes light chain (SEQ ID NO:57) is GACATCCAGATGACCCAGTCTCCATCCTCCTTATCTGCCTCTCTGGGAGAAAGAG TCAGTCTCACTTGTCGGGCAAGTCAGGACATTGGTAGTAGCTTAAACTGGCTTCA GCAGGAACCAGATGGAACTATTAAACGCCTGATCTACGCCACATCCAGTTTAGA TTCTGGTGTCCCCAAAAGGTTCAGTGGCAGTAGGTCTGGGTCAGATTATTCTCTC ACCATCAGCAGCCTTGAGTCTGAAGATTTTGTAGACTATTACTGTCTCCAATATG CTAGTTCTCCTCCGACGTTCGGTGGGGGCACCAAACTGGAAATCAA (SEQ ID NO: 61).

ii. Heavy Chain

One embodiment provides a murine monoclonal antibody or antigen binding fragment thereof that has a heavy chain having at least 50%, 60%, 70%, 80%, 85%, 90%, 95%, 99%, or 100% sequence identity with the following amino acid sequence:

(SEQ ID NO: 62)
EVQLVESGGGLVKPGGTLKLSCAAAGFTFTRYDMSWVRQIPAKRLEWIATI
SGGGGYSFYPDSVKGRFTISRDNAKNTLYLQMSSLRSDDTAVYYCVREDAM
DYWGQGTSVTVSS.

The CDRs are of SEQ ID NO:62 are bolded and underlined and are:

CDR1
(SEQ ID NO: 63)
RYDMS;
CDR2
(SEQ ID NO: 64)
TISGGGGYSFYPDSVKG;
and
CDR3
(SEQ ID NO: 65)
EDAMDY.

Another embodiment provides a nucleic acid encoding heavy chain (SEQ ID NO:62). An exemplary nucleic acid that encodes heavy chain (SEQ ID NO:62) is GAAGTGCAGCTGGTGGAGTCTGGGGGAGGCTTAGTGAAGCCTGGAGGGACCCTG AAACTCTCCTGTGCAGCCGCTGGATTCACTTTCACTAGATATGACATGTCTTGGG TTCGTCAGATTCCGGCGAAGAGGCTGGAGTGGATCGCAACCATTAGTGGTGGTG GTGGTTACAGCTTCTATCCAGACAGTGTGAAGGGCCGATTCACCATCTCCAGAGA CAATGCCAAGAACACCCTGTATCTGCAAATGAGCAGTCTGAGGTCTGATGACAC AGCCGTGTATTACTGTGTAAGGGAGGATGCTATGGACTATTGGGGTCAAGGAAC GTCAGTCACCGTCTCCTCA (SEQ ID NO:66).

One embodiment provides an antibody, preferably a monoclonal antibody, or antigen binding fragment thereof that has a light chain containing CDRs according to SEQ ID Nos: 58, 59, and 60.

One embodiment provides an antibody, preferably a monoclonal antibody, or an antigen binding fragment thereof having a heavy chain containing CDRs according to SEQ ID Nos: 63, 64, and 65.

One embodiment provides an antibody, preferably a monoclonal antibody, or antigen binding fragment thereof that has a light chain containing CDRs according to SEQ ID Nos: 58, 59, and 60 and a heavy chain containing CDRs according to SEQ ID Nos: 63, 64, and 65.

One embodiment provides an antibody, preferably a monoclonal antibody, or an antigen binding fragment thereof having a light chain at least 50%, 60%, 70%, 80%, 85%, 90%, 95%, 99%, or 100% sequence identity to SEQ ID NO:57 and a heavy chain having at least 50%, 60%, 70%, 80%, 85%, 90%, 95%, 99%, or 100% sequence identity to SEQ ID NO:62.

h. K4F4

In one embodiment a murine monoclonal antibody is produced by hybridoma clone K4F4 and consists of two light chains and two heavy chains.

i. Light Chain

One embodiment provides a murine monoclonal antibody or antigen binding fragment thereof that has a light chain having at least 50%, 60%, 70%, 80%, 85%, 90%, 95%, 99%, or 100% sequence identity with the following amino acid sequence:

(SEQ ID NO: 67)
DIQMTQSPSSLSASLGERISLTCRASQDIYGSLNWFQQKPDGTIKLLIYGT
SSLDSGVPKRFSGSRSGSDYSLTISSLESEDFADYYCLQYASFPLTFGAGT
KLELK.

The CDRs are of SEQ ID NO:67 are bolded and underlined and are:

CDR1
(SEQ ID NO: 68)
RASQDIYGSLN;
CDR2
(SEQ ID NO: 69)
GTSSLDS;
and
CDR3
(SEQ ID NO: 70)
LQYASFPLT.

Another embodiment provides a nucleic acid that encodes the light chain (SEQ ID NO:67).

An exemplary nucleic acid that encodes light chain (SEQ ID NO:67) is GACATCCAGATGACCCAGTCTCCATCCTCCTTATCTGCCTCTCTGGGAGAAAGAA TCAGTCTCACTTGCCGGGCAAGTCAGGACATTTATGGTAGCTTAAACTGGTTTCA GCAGAAACCAGATGGAACTATTAAACTCCTGATCTACGGCACATCCAGTTTAGAT TCTGGTGTCCCCAAAAGGTTCAGTGGCAGTAGGTCTGGGTCAGATTATTCTCTCA CCATCAGCAGCCTTGAGTCTGAAGATTTTGCAGACTATTACTGTCTACAATATGC TAGTTTTCCGCTCACGTTCGGTGCTGGGACCAAGCTGGAGCTGAAA (SEQ ID NO:71).

ii. Heavy Chain

One embodiment provides a murine monoclonal antibody or antigen binding fragment thereof that has a heavy chain having at least 50%, 60%, 70%, 80%, 85%, 90%, 95%, 99%, or 100% sequence identity with the following amino acid sequence:

(SEQ ID NO: 72)
QVQLQQPGAEMVRPGASVKLSCKTSGYTFTSYWMHWVKQRPGQGLEW
IGKIDPSDSETHYNQKFKDKATLTVDKSSSTAYIQLNSLTSEDSAVY
YCARGRLLRLRDWFPYWGQGTLVTVSA.

The CDRs are of SEQ ID NO:72 are bolded and underlined and are:

CDR1
(SEQ ID NO: 73)
SYWMH;
CDR2
(SEQ ID NO: 74)
KIDPSDSETHYNQKFKD;
and
CDR3
(SEQ ID NO: 75)
GRLLRLRDWFPY.

Another embodiment provides a nucleic acid encoding heavy chain (SEQ ID NO:72). An exemplary nucleic acid that encodes heavy chain (SEQ ID NO:72) is CAGGTCCAGCTGCAGCAGCCTGGGGCTGAGATGGTGAGGCCTGGGGCTTCAGTG AAGTTGTCCTGCAAGACTTCTGGCTACACCTTCACCAGCTACTGGATGCACTGGG TGAAGCAGAGGCCTGGACAAGGCCTTGAGTGGATTGGTAAGATTGATCCTTCTG ATAGTGAAACTCACTACAATCAAAAGTTCAAGGACAAGGCCACATTGACTGTCG ACAAATCCTCCAGCACAGCCTACATACAGCTCAACAGCCTGACATCTGAAGACT CTGCGGTCTATTACTGTGCAAGAGGAAGGTTACTACGGCTACGTGACTGGTTTCC TTACTGGGGCCAAGGGACTCTGGTCACTGTCTCTGCA (SEQ ID NO:76).

One embodiment provides an antibody, preferably a monoclonal antibody, or antigen binding fragment thereof that has a light chain containing CDRs according to SEQ ID Nos: 68, 69, and 70.

One embodiment provides an antibody, preferably a monoclonal antibody, or an antigen binding fragment thereof having a heavy chain containing CDRs according to SEQ ID Nos: 73, 74, and 75.

One embodiment provides an antibody, preferably a monoclonal antibody, or antigen binding fragment thereof that has a light chain containing CDRs according to SEQ ID Nos: 68, 69, and 70 and a heavy chain containing CDRs according to SEQ ID Nos: 73, 74, and 75.

One embodiment provides an antibody, preferably a monoclonal antibody, or an antigen binding fragment thereof having a light chain at least 50%, 60%, 70%, 80%, 85%, 90%, 95%, 99%, or 100% sequence identity to SEQ ID NO:67 and a heavy chain having at least 50%, 60%, 70%, 80%, 85%, 90%, 95%, 99%, or 100% sequence identity to SEQ ID NO:72.

i. K4F5

In one embodiment a murine monoclonal antibody is produced by hybridoma clone K4F5 and consists of two light chains and two heavy chains.

i. Light Chain

One embodiment provides a murine monoclonal antibody or antigen binding fragment thereof that has a light chain having at least 50%, 60%, 70%, 80%, 85%, 90%, 95%, 99%, or 100% sequence identity with the following amino acid sequence:

(SEQ ID NO: 77)
DIQMTQSPASLSASVGETVTITCRASENIYSCLAWYQQKQGKSPQFL
VHNAKTLAEGVPSRFSGSGSGTLFSLKINSLQPEDFGSYYCQYHYGI
PFTFGSGTKLEIK.

The CDRs are of SEQ ID NO:77 are bolded and underlined and are:

CDR1
(SEQ ID NO: 78)
RASENIYSCLA;
CDR2
(SEQ ID NO: 79)
NAKTLAE;
and
CDR3
(SEQ ID NO: 80)
QYHYGIPFT.

Another embodiment provides a nucleic acid that encodes the light chain (SEQ ID NO:77).

An exemplary nucleic acid that encodes light chain (SEQ ID NO:77) is GACATCCAGATGACTCAGTCTCCAGCCTCCCTATCTGCATCTGTGGGAGAAACTG TCACCATCACATGTCGAGCAAGCGAGAATATTTATAGTTGTTTAGCATGGTATCA GCAGAAACAGGGAAAATCTCCTCAGTTCCTGGTCCATAATGCAAAAACCTTAGC TGAAGGTGTGCCATCAAGGTTCAGTGGCAGTGGATCAGGCACACTATTTTCTCTG AAGATCAACAGCCTGCAGCCTGAAGATTTTGGGAGTTATTACTGTCAATATCATT ATGGCATTCCATTCACGTTCGGCTCGGGGACAAAGTTGGAAATAAAA (SEQ ID NO:81).

ii. Light Chain Variants

One embodiment provides a humanized monoclonal antibody or antigen binding fragment thereof that has a light chain variable domain having at least 50%, 60%, 70%, 80%, 85%, 90%, 95%, 99%, or 100% sequence identity with one of the following amino acid sequences:

K4F5A Light Chain Parent:
(SEQ ID NO: 147)
DIQMTQSPASLSASVGETVTITCRASENIYSALAWYQQKQGKSPQFL
VHNAKTLAEGVPSRFSGSGSGTLFSLKINSLQPEDFGSYYCQYHYGI
PFTFGSGTKLEIK
K4F5A Light Chain VL1
(SEQ ID NO: 121)
DIQMTQSPSSLSASVGDRVTITCRASENIYSALAWYQQKPGKAPKFL
IHNAKTLAEGVPSRFSGSGSGTDFTLTISSLQPEDFATYYCQYHYGI
PFTFGQGTKLEIK
K4F5A Light Chain VL2
(SEQ ID NO: 122)
DIQMTQSPSSLSASVGDRVTITCRASENIYSALAWYQQKPGKAPKFL
IHNVKTLAEGVPSRFSGSGSGTDFTLTISSLQPEDFATYYCQYHYGI
PFTFGQGTKLEIK
K4F5A Light Chain VL3
(SEQ ID NO: 123)
DIQMTQSPSSLSASVGDRVTITCRASENIYSALAWYQQKPGKAPKFL
IHQAKTLAEGVPSRFSGSGSGTDFTLTISSLQPEDFATYYCQYHYGI
PFTFGQGTKLEIK
K4F5A Light Chain VL4
(SEQ ID NO: 124)
DIQMTQSPSSLSASVGDRVTITCRASENIYSALAWYQQKPGKAPKFL
IHSAKTLAEGVPSRFSGSGSGTDFTLTISSLQPEDFATYYCQYHYGI
PFTFGQGTKLEIK

One embodiment provides an antibody or antigen binding fragment thereof that has a human kappa constant region having at least 50%, 60%, 70%, 80%, 85%, 90%, 95%, 99%, or 100% sequence identity with the following amino acid sequences:

(SEQ ID NO: 125)
RTVAAPSVFIFPPSDEQLKSGTASVVCLLNNFYPREAKVQWKVDNAL
QSGNSQESVTEQDSKDSTYSLSSTLTLSKADYEKHKVYACEVTHQGL
SSPVTKSFNRGEC

One embodiment provides an anti-KLRG1 monoclonal antibody or antigen binding fragment thereof that has a humanized light chain having at least 50%, 60%, 70%, 80%, 85%, 90%, 95%, 99%, or 100% sequence identity with one of the following amino acid sequences:

Full length Humanized K4F5A Light Chain Variant 1:
(SEQ ID NO: 126)
DIQMTQSPSSLSASVGDRVTITCRASENIYSALAWYQQKPGKAPKFL
IHNAKTLAEGVPSRFSGSGSGTDFTLTISSLQPEDFATYYCQYHYGI
PFTFGQGTKLEIKRTVAAPSVFIFPPSDEQLKSGTASVVCLLNNFYP
REAKVQWKVDNALQSGNSQESVTEQDSKDSTYSLSSTLTLSKADYEK
HKVYACEVTHQGLSSPVTKSFNRGEC
Full length Humanized K4F5A Light Chain Variant 2:
(SEQ ID NO: 127)
DIQMTQSPSSLSASVGDRVTITCRASENIYSALAWYQQKPGKAPKFL
IHNVKTLAEGVPSRFSGSGSGTDFTLTISSLQPEDFATYYCQYHYGI
PFTFGQGTKLEIKRTVAAPSVFIFPPSDEQLKSGTASVVCLLNNFYP
REAKVQWKVDNALQSGNSQESVTEQDSKDSTYSLSSTLTLSKADYEK
HKVYACEVTHQGLSSPVTKSFNRGEC
Full length Humanized K4F5A Light Chain Variant 3:
(SEQ ID NO: 128)
DIQMTQSPSSLSASVGDRVTITCRASENIYSALAWYQQKPGKAPKFL
IHQAKTLAEGVPSRFSGSGSGTDFTLTISSLQPEDFATYYCQYHYGI
PFTFGQGTKLEIKRTVAAPSVFIFPPSDEQLKSGTASVVCLLNNFYP
REAKVQWKVDNALQSGNSQESVTEQDSKDSTYSLSSTLTLSKADYEK
HKVYACEVTHQGLSSPVTKSFNRGEC
Full Length Humanized K4F5A Light Chain Variant 4:
(SEQ ID NO: 129)
DIQMTQSPSSLSASVGDRVTITCRASENIYSALAWYQQKPGKAPKFL
IHSAKTLAEGVPSRFSGSGSGTDFTLTISSLQPEDFATYYCQYHYGI
PFTFGQGTKLEIKRTVAAPSVFIFPPSDEQLKSGTASVVCLLNNFYP
REAKVQWKVDNALQSGNSQESVTEQDSKDSTYSLSSTLTLSKADYEK
HKVYACEVTHQGLSSPVTKSFNRGEC

iii. Heavy Chain

One embodiment provides a murine monoclonal antibody or antigen binding fragment thereof that has a heavy chain having at least 50%, 60%, 70%, 80%, 85%, 90%, 95%, 99%, or 100% sequence identity with the following amino acid sequence:

(SEQ ID NO: 82)
DVKLVESGGGLVKLGGSLKLSCAASGFTFSRYYMSWVRQTPEKRLEW
VATISISGGNTYYPDTMKGRFTISRDSAKNTLYLQMSSLNSEDTAVY
YCAREGGYGNLWFAYWGQGTLVTVSA.

The CDRs are of SEQ ID NO:82 are bolded and underlined and are:

CDR1
(SEQ ID NO: 83)
RYYMS;
CDR2
(SEQ ID NO: 84)
TISISGGNTYYPDTMKG;
and
CDR3
(SEQ ID NO: 85)
EGGYGNLWFAY.

Another embodiment provides a nucleic acid encoding heavy chain (SEQ ID NO:82. An exemplary nucleic acid that encodes heavy chain (SEQ ID NO:82) is GACGTGAAGCTCGTGGAGTCTGGGGGAGGCTTAGTGAAGCTTGGAGGGTCCCTG AAACTCTCCTGTGCAGCCTCTGGATTCACTTTCAGTCGCTATTACATGTCTTGGGT TCGCCAGACTCCGGAGAAGAGGCTGGAGTGGGTCGCAACCATTAGTATTAGTGG TGGTAACACCTACTACCCAGACACTATGAAGGGCCGATTCACCATCTCCAGAGA CAGTGCCAAGAACACCCTGTACCTGCAAATGAGCAGTCTGAATTCTGAGGACAC AGCCGTGTATTACTGTGCAAGAGAAGGGGGGTATGGTAACCTCTGGTTTGCTTAC TGGGGCCAAGGGACTCTGGTCACTGTCTCTGCA (SEQ ID NO:86).

iv. Humanized Heavy Chain

One embodiment provides a humanized anti-KLRG1 monoclonal antibody or antigen binding fragment thereof that has a heavy chain variable domain having at least 50%, 60%, 70%, 80%, 85%, 90%, 95%, 99%, or 100% sequence identity with one of the following amino acid sequences:

K4F5A Heavy Chain Variant VH1
(SEQ ID NO: 132)
EVQLVESGGGLVKPGGSLRLSCAASGFTFSRYYMSWVRQAPGKGLEW
VATISISGGNTYYPDSVKGRFTISRDNAKNTLYLQMSSLRAEDTAVY
YCAREGGYGNLWFAYWGQGTLVTVSS
K4F5A Heavy Chain Variant VH2
(SEQ ID NO: 133)
EVQLVESGGGLVKPGGSLRLSCAASGFTFSRYYMSWVRQAPGKGLEW
VATISISGGNTYYADSVKGRFTISRDNAKNTLYLQMSSLRAEDTAVY
YCAREGGYGNLWFAYWGQGTLVTVSS
K4F5A Heavy Chain Variant VH3
(SEQ ID NO: 134)
EVQLVESGGGLVKPGGSLRLSCAASGFTFSRYYMSWVRQAPGKGLEW
VATISISGGNVYYPDSVKGRFTISRDNAKNTLYLQMSSLRAEDTAVY
YCAREGGYGNLWFAYWGQGTLVTVSS
K4F5A Heavy Chain Variant VH4
(SEQ ID NO: 135)
EVQLVESGGGLVKPGGSLRLSCAASGFTFSRYYMSWVRQAPGKGLEW
VATISISGGQTYYADSVKGRFTISRDNAKNTLYLQMSSLRAEDTAVY
YCAREGGYGNLWFAYWGQGTLVTVSS
K4F5A Heavy Chain Variant VH5
(SEQ ID NO: 136)
EVQLVESGGGLVKPGGSLRLSCAASGFTFSRYYMSWVRQAPGKGLEW
VATISISGGSTYYADSVKGRFTISRDNAKNTLYLQMSSLRAEDTAVY
YCAREGGYGNLWFAYWGQGTLVTVSS

One embodiment provides a monoclonal antibody or antigen binding fragment thereof having a human hG1 heavy chain constant domain having at least 50%, 60%, 70%, 80%, 85%, 90%, 95%, 99%, or 100% sequence identity with the following amino acid sequence:

(SEQ ID NO: 130)
ASTKGPSVFPLAPSSKSTSGGTAALGCLVKDYFPEPVTVSWNSGALT
SGVHTFPAVLQSSGLYSLSSVVTVPSSSLGTQTYICNVNHKPSNTKV
DKKVEPKSCDKTHTCPPCPAPELLGGPSVFLFPPKPKDTLMISRTPE
VTCVVVDVSHEDPEVKFNWYVDGVEVHNAKTKPREEQYNSTYRVVSV
LTVLHQDWLNGKEYKCKVSNKALPAPIEKTISKAKGQPREPQVYTLP
PSRDELTKNQVSLTCLVKGFYPSDIAVEWESNGQPENNYKTTPPVLD
SDGSFFLYSKLTVDKSRWQQGNVFSCSVMHEALHNHYTQKSLSLSPG
K

Another embodiment provides a monoclonal antibody or antigen binding fragment thereof having a mutant human hG1 heavy chain constant domain having at least 50%, 60%, 70%, 80%, 85%, 90%, 95%, 99%, or 100% sequence identity with the following amino acid sequence:

(SEQ ID NO: 131)
ASTKGPSVFPLAPSSKSTSGGTAALGCLVKDYFPEPVTVSWNSGALT
SGVHTFPAVLQSSGLYSLSSVVTVPSSSLGTQTYICNVNHKPSNTKV
DKKVEPKSCDKTHTCPPCPAPEFEGGPSVFLFPPKPKDTLMISRTPE
VTCVVVDVSHEDPEVKFNWYVDGVEVHNAKTKPREEQYNSTYRVVSV
LTVLHQDWLNGKEYKCKVSNKALPASIEKTISKAKGQPREPQVYTLP
PSRDELTKNQVSLTCLVKGFYPSDIAVEWESNGQPENNYKTTPPVLD
SDGSFFLYSKLTVDKSRWQQGNVFSCSVMHEALHNHYTQKSLSLSPG

The underlined and bolded amino acids represent amino acids that differ between the wild-type and mutant sequences.

One embodiment provides an anti-KLRG1 monoclonal antibody or antigen binding fragment thereof that has a full-length humanized heavy chain having at least 50%, 60%, 70%, 80%, 85%, 90%, 95%, 99%, or 100% sequence identity with one of the following amino acid sequences:

Full-Length Humanized K4F5A Heavy Chain
Variant 1, Wild-type:
(SEQ ID NO: 137)
EVQLVESGGGLVKPGGSLRLSCAASGFTFSRYYMSWVRQAPGKGLEW
VATISISGGNTYYPDSVKGRFTISRDNAKNTLYLQMSSLRAEDTAVY
YCAREGGYGNLWFAYWGQGTLVTVSSASTKGPSVFPLAPSSKSTSGG
TAALGCLVKDYFPEPVTVSWNSGALTSGVHTFPAVLQSSGLYSLSSV
VTVPSSSLGTQTYICNVNHKPSNTKVDKKVEPKSCDKTHTCPPCPAP
ELLGGPSVFLFPPKPKDTLMISRTPEVTCVVVDVSHEDPEVKFNWYV
DGVEVHNAKTKPREEQYNSTYRVVSVLTVLHQDWLNGKEYKCKVSNK
ALPAPIEKTISKAKGQPREPQVYTLPPSRDELTKNQVSLTCLVKGFY
PSDIAVEWESNGQPENNYKTTPPVLDSDGSFFLYSKLTVDKSRWQQG
NVFSCSVMHEALHNHYTQKSLSLSPGK
Full-Length Humanized K4F5A Heavy Chain
Variant 1, mutant:
(SEQ ID NO: 138)
EVQLVESGGGLVKPGGSLRLSCAASGFTFSRYYMSWVRQAPGKGLEW
VATISISGGNTYYPDSVKGRFTISRDNAKNTLYLQMSSLRAEDTAVY
YCAREGGYGNLWFAYWGQGTLVTVSSASTKGPSVFPLAPSSKSTSGG
TAALGCLVKDYFPEPVTVSWNSGALTSGVHTFPAVLQSSGLYSLSSV
VTVPSSSLGTQTYICNVNHKPSNTKVDKKVEPKSCDKTHTCPPCPAP
EFEGGPSVFLFPPKPKDTLMISRTPEVTCVVVDVSHEDPEVKFNWYV
DGVEVHNAKTKPREEQYNSTYRVVSVLTVLHQDWLNGKEYKCKVSNK
ALPASIEKTISKAKGQPREPQVYTLPPSRDELTKNQVSLTCLVKGFY
PSDIAVEWESNGQPENNYKTTPPVLDSDGSFFLYSKLTVDKSRWQQG
NVFSCSVMHEALHNHYTQKSLSLSPG
Full-Length Humanized K4F5A Heavy Chain
Variant 2, wild type:
(SEQ ID NO: 139)
EVQLVESGGGLVKPGGSLRLSCAASGFTFSRYYMSWVRQAPGKGLEW
VATISISGGNTYYADSVKGRFTISRDNAKNTLYLQMSSLRAEDTAVY
YCAREGGYGNLWFAYWGQGTLVTVSSASTKGPSVFPLAPSSKSTSGG
TAALGCLVKDYFPEPVTVSWNSGALTSGVHTFPAVLQSSGLYSLSSV
VTVPSSSLGTQTYICNVNHKPSNTKVDKKVEPKSCDKTHTCPPCPAP
ELLGGPSVFLFPPKPKDTLMISRTPEVTCVVVDVSHEDPEVKFNWYV
DGVEVHNAKTKPREEQYNSTYRVVSVLTVLHQDWLNGKEYKCKVSNK
ALPAPIEKTISKAKGQPREPQVYTLPPSRDELTKNQVSLTCLVKGFY
PSDIAVEWESNGQPENNYKTTPPVLDSDGSFFLYSKLTVDKSRWQQG
NVFSCSVMHEALHNHYTQKSLSLSPGK
Full-Length Humanized K4F5A Heavy Chain
Variant 2, mutant:
(SEQ ID NO: 140)
EVQLVESGGGLVKPGGSLRLSCAASGFTFSRYYMSWVRQAPGKGLEW
VATISISGGNTYYADSVKGRFTISRDNAKNTLYLQMSSLRAEDTAVY
YCAREGGYGNLWFAYWGQGTLVTVSSASTKGPSVFPLAPSSKSTSGG
TAALGCLVKDYFPEPVTVSWNSGALTSGVHTFPAVLQSSGLYSLSSV
VTVPSSSLGTQTYICNVNHKPSNTKVDKKVEPKSCDKTHTCPPCPAP
EFEGGPSVFLFPPKPKDTLMISRTPEVTCVVVDVSHEDPEVKFNWYV
DGVEVHNAKTKPREEQYNSTYRVVSVLTVLHQDWLNGKEYKCKVSNK
ALPASIEKTISKAKGQPREPQVYTLPPSRDELTKNQVSLTCLVKGFY
PSDIAVEWESNGQPENNYKTTPPVLDSDGSFFLYSKLTVDKSRWQQG
NVFSCSVMHEALHNHYTQKSLSLSPG
Full-Length Humanized K4F5A Heavy Chain
Variant 3, wild-type:
(SEQ ID NO: 141)
EVQLVESGGGLVKPGGSLRLSCAASGFTFSRYYMSWVRQAPGKGLEW
VATISISGGNVYYPDSVKGRFTISRDNAKNTLYLQMSSLRAEDTAVY
YCAREGGYGNLWFAYWGQGTLVTVSSASTKGPSVFPLAPSSKSTSGG
TAALGCLVKDYFPEPVTVSWNSGALTSGVHTFPAVLQSSGLYSLSSV
VTVPSSSLGTQTYICNVNHKPSNTKVDKKVEPKSCDKTHTCPPCPAP
ELLGGPSVFLFPPKPKDTLMISRTPEVTCVVVDVSHEDPEVKFNWYV
DGVEVHNAKTKPREEQYNSTYRVVSVLTVLHQDWLNGKEYKCKVSNK
ALPAPIEKTISKAKGQPREPQVYTLPPSRDELTKNQVSLTCLVKGFY
PSDIAVEWESNGQPENNYKTTPPVLDSDGSFFLYSKLTVDKSRWQQG
NVFSCSVMHEALHNHYTQKSLSLSPGK
Full-Length Humanized K4F5A Heavy Chain
Variant 3, mutant:
(SEQ ID NO: 142)
EVQLVESGGGLVKPGGSLRLSCAASGFTFSRYYMSWVRQAPGKGLEW
VATISISGGNVYYPDSVKGRFTISRDNAKNTLYLQMSSLRAEDTAVY
YCAREGGYGNLWFAYWGQGTLVTVSSASTKGPSVFPLAPSSKSTSGG
TAALGCLVKDYFPEPVTVSWNSGALTSGVHTFPAVLQSSGLYSLSSV
VTVPSSSLGTQTYICNVNHKPSNTKVDKKVEPKSCDKTHTCPPCPAP
EFEGGPSVFLFPPKPKDTLMISRTPEVTCVVVDVSHEDPEVKFNWYV
DGVEVHNAKTKPREEQYNSTYRVVSVLTVLHQDWLNGKEYKCKVSNK
ALPASIEKTISKAKGQPREPQVYTLPPSRDELTKNQVSLTCLVKGFY
PSDIAVEWESNGQPENNYKTTPPVLDSDGSFFLYSKLTVDKSRWQQG
NVFSCSVMHEALHNHYTQKSLSLSPG
Full-Length Humanized K4F5A Heavy Chain
Variant 4, wild type:
(SEQ ID NO: 143)
EVQLVESGGGLVKPGGSLRLSCAASGFTFSRYYMSWVRQAPGKGLEW
VATISISGGQTYYADSVKGRFTISRDNAKNTLYLQMSSLRAEDTAVY
YCAREGGYGNLWFAYWGQGTLVTVSSASTKGPSVFPLAPSSKSTSGG
TAALGCLVKDYFPEPVTVSWNSGALTSGVHTFPAVLQSSGLYSLSSV
VTVPSSSLGTQTYICNVNHKPSNTKVDKKVEPKSCDKTHTCPPCPAP
ELLGGPSVFLFPPKPKDTLMISRTPEVTCVVVDVSHEDPEVKFNWYV
DGVEVHNAKTKPREEQYNSTYRVVSVLTVLHQDWLNGKEYKCKVSNK
ALPAPIEKTISKAKGQPREPQVYTLPPSRDELTKNQVSLTCLVKGFY
PSDIAVEWESNGQPENNYKTTPPVLDSDGSFFLYSKLTVDKSRWQQG
NVFSCSVMHEALHNHYTQKSLSLSPGK
Full-Length Humanized K4F5A Heavy Chain
Variant 4, mutant:
(SEQ ID NO: 144)
EVQLVESGGGLVKPGGSLRLSCAASGFTFSRYYMSWVRQAPGKGLEW
VATISISGGQTYYADSVKGRFTISRDNAKNTLYLQMSSLRAEDTAVY
YCAREGGYGNLWFAYWGQGTLVTVSSASTKGPSVFPLAPSSKSTSGG
TAALGCLVKDYFPEPVTVSWNSGALTSGVHTFPAVLQSSGLYSLSSV
VTVPSSSLGTQTYICNVNHKPSNTKVDKKVEPKSCDKTHTCPPCPAP
EFEGGPSVFLFPPKPKDTLMISRTPEVTCVVVDVSHEDPEVKFNWYV
DGVEVHNAKTKPREEQYNSTYRVVSVLTVLHQDWLNGKEYKCKVSNK
ALPASIEKTISKAKGQPREPQVYTLPPSRDELTKNQVSLTCLVKGFY
PSDIAVEWESNGQPENNYKTTPPVLDSDGSFFLYSKLTVDKSRWQQG
NVFSCSVMHEALHNHYTQKSLSLSPG
Full-Length Humanized K4F5A Heavy Chain
Variant 4, wild type:
(SEQ ID NO: 145)
EVQLVESGGGLVKPGGSLRLSCAASGFTFSRYYMSWVRQAPGKGLEW
VATISISGGSTYYADSVKGRFTISRDNAKNTLYLQMSSLRAEDTAVY
YCAREGGYGNLWFAYWGQGTLVTVSSASTKGPSVFPLAPSSKSTSGG
TAALGCLVKDYFPEPVTVSWNSGALTSGVHTFPAVLQSSGLYSLSSV
VTVPSSSLGTQTYICNVNHKPSNTKVDKKVEPKSCDKTHTCPPCPAP
ELLGGPSVFLFPPKPKDTLMISRTPEVTCVVVDVSHEDPEVKFNWYV
DGVEVHNAKTKPREEQYNSTYRVVSVLTVLHQDWLNGKEYKCKVSNK
ALPAPIEKTISKAKGQPREPQVYTLPPSRDELTKNQVSLTCLVKGFY
PSDIAVEWESNGQPENNYKTTPPVLDSDGSFFLYSKLTVDKSRWQQG
NVFSCSVMHEALHNHYTQKSLSLSPGK
Full-Length Humanized K4F5A Heavy Chain
Variant 4, mutant:
(SEQ ID NO: 146)
EVQLVESGGGLVKPGGSLRLSCAASGFTFSRYYMSWVRQAPGKGLEW
VATISISGGSTYYADSVKGRFTISRDNAKNTLYLQMSSLRAEDTAVY
YCAREGGYGNLWFAYWGQGTLVTVSSASTKGPSVFPLAPSSKSTSGG
TAALGCLVKDYFPEPVTVSWNSGALTSGVHTFPAVLQSSGLYSLSSV
VTVPSSSLGTQTYICNVNHKPSNTKVDKKVEPKSCDKTHTCPPCPAP
EFEGGPSVFLFPPKPKDTLMISRTPEVTCVVVDVSHEDPEVKFNWYV
DGVEVHNAKTKPREEQYNSTYRVVSVLTVLHQDWLNGKEYKCKVSNK
ALPASIEKTISKAKGQPREPQVYTLPPSRDELTKNQVSLTCLVKGFY
PSDIAVEWESNGQPENNYKTTPPVLDSDGSFFLYSKLTVDKSRWQQG
NVFSCSVMHEALHNHYTQKSLSLSPG

Bolded and underlined amino acids represent amino acids that differ between the wild-type and mutant counterparts.

One embodiment provides an antibody, preferably a monoclonal antibody, or antigen binding fragment thereof that has a light chain containing CDRs according to SEQ ID Nos: 78, 79, and 80.

One embodiment provides an anti-KLRG1 antibody, preferably a monoclonal antibody, or an antigen binding fragment thereof having a heavy chain containing CDRs according to SEQ ID Nos: 83, 84, and 85.

One embodiment provides an anti-KLRG1 antibody, preferably a monoclonal antibody, or antigen binding fragment thereof that has a light chain containing CDRs according to SEQ ID Nos: 78, 79, and 80 and a heavy chain containing CDRs according to SEQ ID Nos: 83, 84, and 85.

One embodiment provides an anti-KLRG1 antibody, preferably a monoclonal antibody, or an antigen binding fragment thereof having a light chain at least 50%, 60%, 70%, 80%, 85%, 90%, 95%, 99%, or 100% sequence identity to SEQ ID NO:77 and a heavy chain having at least 50%, 60%, 70%, 80%, 85%, 90%, 95%, 99%, or 100% sequence identity to SEQ ID NO:82.

Another embodiment provides an anti-KLRG1 antibody preferably a monoclonal antibody, or an antigen binding fragment thereof having a light chain variable domain having at least 50%, 60%, 70%, 80%, 85%, 90%, 95%, 99%, or 100% sequence identity to any one of SEQ ID NOs:121, 122, 123, 124, or 125, and a heavy chain variable domain having at least 50%, 60%, 70%, 80%, 85%, 90%, 95%, 99%, or 100% sequence identity to any one of SEQ ID NOs:132, 133, 134, 135, or 136.

One embodiment provides an anti-KLRG1 antibody or antigen binding fragment thereof having a light chain having an amino acid sequence according to any one of SEQ ID NOs:126, 127, 128, or 129, and a heavy chain having an amino acid sequence according to any one of SEQ ID NOs: 137, 139, 141, 143, or 145.

Another embodiment provides an anti-KLRG1 antibody or antigen binding fragment thereof having a light chain having an amino acid sequence according to any one of SEQ ID NOs: 126, 127, 128, or 129, and a heavy chain having an amino acid sequence according to any one of SEQ ID NOs: 138, 140, 142, 144, or 146.

One embodiment provides an anti-KLRG1 antibody or antigen binding fragment thereof having two light chains and two heavy chains, wherein the two light chains include a polypeptide selected from the group consisting of SEQ ID NO: 126, 127, 128, or 129, or a variant thereof having at least 50%, 60%, 70%, 80%, 85%, 90%, 95%, 99%, or more sequence identity to SEQ ID NO: 126, 127, 128, or 129, and the two heavy chains include a polypeptide selected from the group consisting of SEQ ID NO: 137, 139, 141, 143, or 145, or a variant thereof having at least 50%, 60%, 70%, 80%, 85%, 90%, 95%, 99%, or more sequence identity to SEQ ID NO:137, 139, 141, 143, or 145, and wherein the antibody or antigen binding fragment thereof binds to KLRG1.

Another embodiment provides an antibody or antigen binding fragment thereof having two light chains and two heavy chains, wherein the two light chains include a polypeptide selected from the group consisting of SEQ ID NO: 126, 127, 128, or 129, or a variant thereof having at least 50%, 60%, 70%, 80%, 85%, 90%, 95%, 99%, or more sequence identity to SEQ ID NO: 126, 127, 128, or 129, and the two heavy chains include a polypeptide selected from the group consisting of SEQ ID NO: 138, 140, 142, 144, or 146, or a variant thereof having at least 50%, 60%, 70%, 80%, 85%, 90%, 95%, 99%, or more sequence identity to SEQ ID NO: 138, 140, 142, 144, or 146, and wherein the antibody or antigen binding fragment thereof binds to KLRG1.

j. K7C12

In one embodiment a murine monoclonal antibody is produced by hybridoma clone K7C12 and consists of two light chains and two heavy chains.

i. Light Chain

One embodiment provides a murine monoclonal antibody or antigen binding fragment thereof that has a light chain having at least 50%, 60%, 70%, 80%, 85%, 90%, 95%, 99%, or 100% sequence identity with the following amino acid sequence:

(SEQ ID NO: 87)
DIQMTQSPSSLSASLGERVSLTCRASQDIGSSLNWLQQEPDGTIKRL
IYATSSLDSGVPKRFSGSRSGSDYSLTISSLESEDFVDYYCLQYAGS
PPTFGSGTKLELK.

The CDRs are of SEQ ID NO:87 are bolded and underlined and are:

CDR1
(SEQ ID NO: 58)
RASQDIGSSLN;
CDR2
(SEQ ID NO: 59)
ATSSLDS;
and
CDR3
(SEQ ID NO: 88)
LQYAGSPPT.

Another embodiment provides a nucleic acid that encodes the light chain (SEQ ID NO:87).

An exemplary nucleic acid that encodes light chain (SEQ ID NO:87) is GACATCCAGATGACCCAGTCTCCATCCTCCTTATCTGCCTCTCTGGGAGAAAGAG TCAGTCTCACTTGTCGGGCAAGTCAGGACATTGGTAGTAGCTTAAACTGGCTTCA GCAGGAACCAGATGGAACTATTAAACGCCTGATCTACGCCACATCCAGTTTAGA TTCTGGTGTCCCCAAAAGGTTCAGTGGCAGTAGGTCTGGGTCAGATTATTCTCTC ACCATCAGCAGCCTTGAGTCTGAAGATTTTGTAGACTATTACTGTCTACAATATG CTGGTTCTCCTCCCACGTTCGGTTCTGGGACCAAGCTGGAGCTGAAA (SEQ ID NO:89).

ii. Heavy Chain

One embodiment provides a murine monoclonal antibody or antigen binding fragment thereof that has a heavy chain having at least 50%, 60%, 70%, 80%, 85%, 90%, 95%, 99%, or 100% sequence identity with the following amino acid sequence:

(SEQ ID NO: 90)
EVQLVESGGGLVKPGGSLKLSCAASGFTFSNYYMSWVRQTPAKRLEWVAT
ISGGGGYTFYPDSLKGRFTISRDNAKNTLYLQMSSLRSEDTAMYYCARDQ
DYGTIYYAMDYWGQGTSVTVSS.

The CDRs are of SEQ ID NO:90 are bolded and underlined and are:

CDR1
(SEQ ID NO: 91)
NYYMS;
CDR2
(SEQ ID NO: 92)
TISGGGGYTFYPDSLKG;
and
CDR3
(SEQ ID NO: 93)
DQDYGTIYYAMDY.

Another embodiment provides a nucleic acid encoding heavy chain (SEQ ID NO:90).

An exemplary nucleic acid that encodes heavy chain (SEQ ID NO:90) is GAAGTGCAGCTGGTGGAGTCTGGGGGAGGCTTAGTGAAGCCTGGAGGGTCCCTG AAACTCTCCTGTGCAGCCTCTGGATTCACTTTCAGTAACTATTACATGTCTTGGGT TCGCCAGACTCCGGCGAAGAGGCTGGAGTGGGTCGCAACCATTAGTGGTGGTGG TGGTTACACCTTCTATCCAGACAGTTTGAAGGGCCGATTCACCATCTCCAGAGAC AATGCCAAGAACACCCTATACCTGCAAATGAGCAGTCTGAGGTCTGAGGACACA GCCATGTATTACTGTGCAAGGGATCAGGACTACGGTACTATTTACTATGCTATGG ACTACTGGGGTCAAGGAACCTCAGTCACCGTCTCCTCA (SEQ ID NO:94).

One embodiment provides an antibody, preferably a monoclonal antibody, or antigen binding fragment thereof that has a light chain containing CDRs according to SEQ ID Nos: 58, 59, and 88.

One embodiment provides an antibody, preferably a monoclonal antibody, or an antigen binding fragment thereof having a heavy chain containing CDRs according to SEQ ID Nos: 91, 92, and 93.

One embodiment provides an antibody, preferably a monoclonal antibody, or antigen binding fragment thereof that has a light chain containing CDRs according to SEQ ID Nos 58, 59, and 88 and a heavy chain containing CDRs according to SEQ ID Nos: 91, 92, and 93.

One embodiment provides an antibody, preferably a monoclonal antibody, or an antigen binding fragment thereof having a light chain at least 50%, 60%, 70%, 80%, 85%, 90%, 95%, 99%, or 100% sequence identity to SEQ ID NO:87 and a heavy chain having at least 50%, 60%, 70%, 80%, 85%, 90%, 95%, 99%, or 100% sequence identity to SEQ ID NO:90.

k. K8F2

In one embodiment a murine monoclonal antibody is produced by hybridoma clone K8F2 and consists of two light chains and two heavy chains.

i. Light Chain

One embodiment provides a murine monoclonal antibody or antigen binding fragment thereof that has a light chain having at least 50%, 60%, 70%, 80%, 85%, 90%, 95%, 99%, or 100% sequence identity with the following amino acid sequence:

(SEQ ID NO: 95)
DVVMTQTPLTLSVTIGQPASISCKSSQSLLYSDGKTYLNWLLQSPGQSPK
LLIYLVSELESGVPDRFSGSGSGTDFTLKISRVEAEDLGVYYCVQGTHFP
WTFGGGTKLEIK.

The CDRs are of SEQ ID NO:95 are bolded and underlined and are:

CDR1
(SEQ ID NO: 96)
KSSQSLLYSDGKTYLN;
CDR2
(SEQ ID NO: 97)
LVSELES;
and
CDR3
(SEQ ID NO: 98)
VQGTHFPWT.

Another embodiment provides a nucleic acid that encodes the light chain (SEQ ID NO:95).

An exemplary nucleic acid that encodes light chain (SEQ ID NO:95) is GATGTTGTGATGACCCAGACTCCACTCACTTTGTCTGTTACCATTGGACAGCCAG CTTCCATTTCTTGCAAGTCAAGTCAGAGCCTCTTATATAGTGATGGAAAAACCTA TTTGAATTGGTTATTACAGAGTCCAGGCCAGTCTCCAAAGCTCCTAATCTATCTG GTGTCTGAACTGGAATCTGGAGTCCCTGACAGATTCAGTGGCAGTGGATCAGGG ACAGATTTTACACTGAAAATCAGCAGAGTGGAGGCTGAGGATTTGGGAGTTTATT ACTGCGTGCAAGGTACACATTTCCCGTGGACGTTCGGTGGAGGCACCAAGCTGG AAATCAAA (SEQ ID NO:99).

ii. Heavy Chain

One embodiment provides a murine monoclonal antibody or antigen binding fragment thereof that has a heavy chain having at least 50%, 60%, 70%, 80%, 85%, 90%, 95%, 99%, or 100% sequence identity with the following amino acid sequence:

(SEQ ID NO: 100)
EVQLQQSGTVLARPGASVKMSCKASGYTFTNYWMHWVKQRPGQGLEWIGA
IYPGNSDTTYNQKFKGKAKLTAVTSATTAYMELSSLTNEGSAVYYCTREG
DYVYAMDYWGQGTSVTVSS.

The CDRs are of SEQ ID NO:100 are bolded and underlined and are:

CDR1
(SEQ ID NO: 101)
NYVVMH;
CDR2
(SEQ ID NO: 102)
AIYPGNSDTTYNQKFKG;
and
CDR3
(SEQ ID NO: 103)
EGDYVYAMDY.

Another embodiment provides a nucleic acid encoding heavy chain (SEQ ID NO:100).

An exemplary nucleic acid that encodes heavy chain (SEQ ID NO:100) is GAGGTTCAGCTCCAGCAGTCTGGGACTGTGCTGGCAAGGCCTGGGGCTTCAGTG AAGATGTCCTGCAAGGCTTCTGGCTACACCTTTACCAACTACTGGATGCACTGGG TAAAACAGAGGCCTGGACAGGGTCTGGAATGGATTGGCGCTATTTATCCTGGAA ATAGTGATACTACCTACAACCAGAAGTTCAAGGGCAAGGCCAAACTGACTGCAG TCACATCTGCCACCACTGCCTACATGGAACTCAGCAGCCTGACAAATGAGGGCTC TGCGGTCTATTACTGTACAAGAGAGGGTGATTACGTCTATGCTATGGACTACTGG GGTCAAGGAACCTCAGTCACCGTCTCCTCA (SEQ ID NO:104).

One embodiment provides an antibody, preferably a monoclonal antibody, or antigen binding fragment thereof that has a light chain containing CDRs according to SEQ ID Nos: 96, 97, and 98.

One embodiment provides an antibody, preferably a monoclonal antibody, or an antigen binding fragment thereof having a heavy chain containing CDRs according to SEQ ID Nos: 101, 102, and 103.

One embodiment provides an antibody, preferably a monoclonal antibody, or antigen binding fragment thereof that has a light chain containing CDRs according to SEQ ID Nos: 96, 97, and 98 and a heavy chain containing CDRs according to SEQ ID 101, 102, and 103.

One embodiment provides an antibody, preferably a monoclonal antibody, or an antigen binding fragment thereof having a light chain at least 50%, 60%, 70%, 80%, 85%, 90%, 95%, 99%, or 100% sequence identity to SEQ ID NO:95 and a heavy chain having at least 50%, 60%, 70%, 80%, 85%, 90%, 95%, 99%, or 100% sequence identity to SEQ ID NO:100.

1. K9H1

In one embodiment a murine monoclonal antibody is produced by hybridoma clone K9H1 and consists of two light chains and two heavy chains.

i. Light Chain

One embodiment provides a murine monoclonal antibody or antigen binding fragment thereof that has a light chain having at least 50%, 60%, 70%, 80%, 85%, 90%, 95%, 99%, or 100% sequence identity with the following amino acid sequence:

(SEQ ID NO: 105)
DIVMTQSQKFMSTSVGDRVSITGKASQNYGTALAWYQQKPGQSPKWYSAS
NRYTGVPDRFTGSGSGTDFTLTISNMQSEDLADYFCQQYSSYPLTIGAGT
KLELR.

The CDRs are of SEQ ID NO:105 are bolded and underlined and are:

CDR1
(SEQ ID NO: 106)
KASQNVGTALA;
CDR2
(SEQ ID NO: 26)
SASNRYT;
and
CDR3
(SEQ ID NO: 7)
QQYSSYPLT.

Another embodiment provides a nucleic acid that encodes the light chain (SEQ ID NO:105).

An exemplary nucleic acid that encodes light chain (SEQ ID NO:105) is GACATTGTGATGACCCAGTCTCAAAAATTCATGTCCACATCAGTAGGAGACAGG GTCAGCATCACCGGCAAGGCCAGTCAGAATGTGGGTACTGCTTTAGCCTGGTATC AACAGAAACCAGGACAATCTCCTAAACTACTGATTTACTCGGCATCCAATCGGTA CACTGGAGTCCCTGATCGCTTCACAGGCAGTGGATCTGGGACAGATTTCACTCTC ACCATCAGCAATATGCAGTCTGAAGACCTGGCAGATTATTTCTGTCAGCAATATA GTAGCTATCCTCTCACGATCGGTGCTGGGACCAAGCTGGAGCTGAGA (SEQ ID NO:107).

ii. Heavy Chain

One embodiment provides a murine monoclonal antibody or antigen binding fragment thereof that has a heavy chain having at least 50%, 60%, 70%, 80%, 85%, 90%, 95%, 99%, or 100% sequence identity with the following amino acid sequence:

(SEQ ID NO: 108)
EVQLQQSGPELVKPGASVKMSCKASGYTFTNYVMHWVKQKPGQGLEWIGY
IIPYNDGTIVNEKFRGKATLTSDKFSSTAYMELSSLTSEDSAVYYCARGD
NDSDGDAMDYWGQGTSVTVSS.

The CDRs are of SEQ ID NO:108 are bolded and underlined and are:

CDR1
(SEQ ID NO: 147)
NYVMH;
CDR2
(SEQ ID NO: 109)
YIIPYNDGTIYNEKFRG;
and
CDR3
(SEQ ID NO: 110)
GDNDSDGDAMDY.

Another embodiment provides a nucleic acid encoding heavy chain (SEQ ID NO:108).

An exemplary nucleic acid that encodes heavy chain (SEQ ID NO:108) is GAGGTCCAGCTGCAGCAGTCTGGACCTGAGCTGGTAAAGCCTGGGGCTTCAGTG AAGATGTCCTGCAAGGCTTCTGGATACACATTCACTAACTATGTTATGCACTGGG TGAAGCAGAAGCCTGGGCAGGGCCTTGAGTGGATTGGATATATTATTCCTTACAA TGATGGTACTATTTACAATGAGAAATTCAGAGGCAAGGCCACACTGACTTCAGA CAAATTCTCCAGCACAGCCTACATGGAGCTCAGCAGCCTGACCTCTGAGGACTCT GCGGTCTATTACTGTGCAAGAGGGGATAACGACTCTGATGGGGATGCTATGGAC TACTGGGGTCAAGGAACCTCAGTCACCGTCTCCTCA (SEQ ID NO:111).

One embodiment provides an antibody, preferably a monoclonal antibody, or antigen binding fragment thereof that has a light chain containing CDRs according to SEQ ID Nos: 106, 26, and 7.

One embodiment provides an antibody, preferably a monoclonal antibody, or an antigen binding fragment thereof having a heavy chain containing CDRs according to SEQ ID Nos: 109, 110 and 147.

One embodiment provides an antibody, preferably a monoclonal antibody, or antigen binding fragment thereof that has a light chain containing CDRs according to SEQ ID Nos: 106, 26, and 7 and a heavy chain containing CDRs according to SEQ ID 109, 110 and 147.

One embodiment provides an antibody, preferably a monoclonal antibody, or an antigen binding fragment thereof having a light chain at least 50%, 60%, 70%, 80%, 85%, 90%, 95%, 99%, or 100% sequence identity to SEQ ID NO:105 and a heavy chain having at least 50%, 60%, 70%, 80%, 85%, 90%, 95%, 99%, or 100% sequence identity to SEQ ID NO:108.

m. K10D5

In one embodiment a murine monoclonal antibody is produced by hybridoma clone K10D5 and consists of two light chains and two heavy chains.

i. Light Chain

One embodiment provides a murine monoclonal antibody or antigen binding fragment thereof that has a light chain having at least 50%, 60%, 70%, 80%, 85%, 90%, 95%, 99%, or 100% sequence identity with the following amino acid sequence:

(SEQ ID NO: 112)
DIQMTQSPSSLSASLGERVSLTCRASQDIGGSLNWLQQEPDGTIKRLIYA
TSSLDSGVPKRFSGSRSGSDYSLTISSLESEDFVYYYCLQYASSPLTFGA
GTKLELK.

The CDRs are of SEQ ID NO:112 are bolded and underlined and are:

CDR1
(SEQ ID NO: 113)
RASQDIGGSLN;
CDR2
(SEQ ID NO: 59)
ATSSLDS;
and
CDR3
(SEQ ID NO: 114)
LQYASSPLT.

Another embodiment provides a nucleic acid that encodes the light chain (SEQ ID NO:112).

An exemplary nucleic acid that encodes light chain (SEQ ID NO:112) is GACATCCAGATGACCCAGTCTCCATCCTCCTTATCTGCCTCTCTGGGAGAAAGAG TCAGTCTCACTTGTCGGGCAAGTCAGGACATTGGTGGTAGCTTAAACTGGCTTCA GCAGGAACCAGATGGAACTATTAAACGCCTGATCTACGCCACATCCAGTTTAGA TTCTGGTGTCCCCAAAAGGTTCAGTGGCAGTAGGTCTGGGTCAGATTATTCTCTC ACCATCAGCAGCCTTGAGTCTGAAGATTTTGTATACTATTACTGTCTACAATATG CTAGTTCTCCGCTCACGTTCGGTGCTGGGACCAAGCTGGAGCTGAAA (SEQ ID NO:115).

ii. Heavy Chain

One embodiment provides a murine monoclonal antibody or antigen binding fragment thereof that has a heavy chain having at least 50%, 60%, 70%, 80%, 85%, 90%, 95%, 99%, or 100% sequence identity with the following amino acid sequence:

(SEQ ID NO: 116)
DVKLVESGGGLVKLGGSLKLSCAASGFTFSSYYMSWVRQTPEKRLEWVAT
ISNSGRSTYYPDTVKGRFTISRDSAKNTLYLQMSSLNSEDTAVYFCARDR
DYGYTYEALDYWGQGTSVTVSS.

The CDRs are of SEQ ID NO:116 are bolded and underlined and are:

CDR1
(SEQ ID NO: 117)
SYYMS;
CDR2
(SEQ ID NO: 118)
TISNSGRSTYYPDTVKG;
and
CDR3
(SEQ ID NO: 119)
DRDYGYTYEALDY.

Another embodiment provides a nucleic acid encoding heavy chain (SEQ ID NO:116).

An exemplary nucleic acid that encodes heavy chain (SEQ ID NO:116) is GACGTGAAGCTCGTGGAGTCTGGGGGAGGCTTAGTGAAGCTTGGAGGGTCCCTG AAACTCTCCTGTGCAGCCTCTGGATTCACTTTCAGTAGCTATTACATGTCTTGGGT TCGCCAGACTCCGGAGAAGAGGCTGGAGTGGGTCGCAACCATTAGTAATAGTGG TCGTAGTACCTACTATCCAGACACTGTGAAGGGCCGATTCACCATCTCCAGAGAC AGTGCCAAGAACACCCTGTATCTGCAAATGAGCAGTCTGAATTCTGAGGACACA GCCGTGTATTTCTGTGCAAGAGATCGGGACTACGGTTATACCTACGAAGCTTTGG ACTACTGGGGTCAAGGAACCTCAGTCACCGTCTCCTCA (SEQ ID NO:120).

One embodiment provides an antibody, preferably a monoclonal antibody, or antigen binding fragment thereof that has a light chain containing CDRs according to SEQ ID Nos: 113, 59, and 114.

One embodiment provides an antibody, preferably a monoclonal antibody, or an antigen binding fragment thereof having a heavy chain containing CDRs according to SEQ ID Nos: 117, 118, and 119.

One embodiment provides an antibody, preferably a monoclonal antibody, or antigen binding fragment thereof that has a light chain containing CDRs according to SEQ ID Nos: 113, 59, and 114 and a heavy chain containing CDRs according to SEQ ID 117, 118, and 119.

One embodiment provides an antibody, preferably a monoclonal antibody, or an antigen binding fragment thereof having a light chain at least 50%, 60%, 70%, 80%, 85%, 90%, 95%, 99%, or 100% sequence identity to SEQ ID NO:112 and a heavy chain having at least 50%, 60%, 70%, 80%, 85%, 90%, 95%, 99%, or 100% sequence identity to SEQ ID NO:116.

2. Antibody Compositions

The disclosed KLRG1-binding molecules can be antibodies or antigen binding fragments thereof. The disclosed antibodies and antigen binding fragments thereof include whole immunoglobulin (i.e., an intact antibody) of any class, fragments thereof, and synthetic proteins containing at least the antigen binding variable domain of an antibody. In some embodiments, the disclosed molecule contains both an antibody light chain as well as at least the variable domain of an antibody heavy chain. In other embodiments, such molecules can further include one or more of the CH₁, hinge, CH₂, CH₃, and CH₄ regions of the heavy chain (especially, the CH₁ and hinge regions, or the CH₁, hinge and CH₂ regions, or the CH₁, hinge, CH₂ and CH₃ regions). The antibody can be selected from any class of immunoglobulins, including IgM, IgG, IgD, IgA and IgE, and any isotype, including IgG₁, IgG₂, IgG₃ and IgG₄. In some embodiments, the constant domain is a complement fixing constant domain where it is desired that the antibody exhibit cytotoxic activity, and the class is typically IgG₁. In other embodiments, where such cytotoxic activity is not desirable, the constant domain can be of the IgG₂ or IgG₄ class. The antibody can include sequences from more than one class or isotype, and selecting particular constant domains to optimize desired effector functions is within the ordinary skill in the art.

The variable domains differ in sequence among antibodies and are used in the binding and specificity of each particular antibody for its particular antigen. However, the variability is not usually evenly distributed through the variable domains of antibodies. It is typically concentrated in three segments called complementarity determining regions (CDRs) or hypervariable regions both in the light chain and the heavy chain variable domains. The more highly conserved portions of the variable domains are called the framework (FR). The variable domains of native heavy and light chains each comprise four FR regions, largely adopting a beta-sheet configuration, connected by three CDRs, which form loops connecting, and in some cases forming part of, the beta-sheet structure. The CDRs in each chain are held together in close proximity by the FR regions and, with the CDRs from the other chain, contribute to the formation of the antigen binding site of antibodies.

Also disclosed are fragments of antibodies which have bioactivity. The fragments, whether attached to other sequences or not, include insertions, deletions, substitutions, or other selected modifications of particular regions or specific amino acids residues, provided the activity of the fragment is not significantly altered or impaired compared to the non-modified antibody or antibody fragment.

Techniques can also be adapted for the production of single-chain antibodies specific to KLRG1. Methods for the production of single-chain antibodies are well known to those of skill in the art. A single chain antibody can be created by fusing together the variable domains of the heavy and light chains using a short peptide linker, thereby reconstituting an antigen binding site on a single molecule. Single-chain antibody variable fragments (scFvs) in which the C-terminus of one variable domain is tethered to the N-terminus of the other variable domain via a 15 to 25 amino acid peptide or linker have been developed without significantly disrupting antigen binding or specificity of the binding. The linker is chosen to permit the heavy chain and light chain to bind together in their proper conformational orientation.

Divalent single-chain variable fragments (di-scFvs) can be engineered by linking two scFvs. This can be done by producing a single peptide chain with two VH and two VL regions, yielding tandem scFvs. ScFvs can also be designed with linker peptides that are too short for the two variable regions to fold together (about five amino acids), forcing scFvs to dimerize. This type is known as diabodies. Diabodies have been shown to have dissociation constants up to 40-fold lower than corresponding scFvs, meaning that they have a much higher affinity to their target. Still shorter linkers (one or two amino acids) lead to the formation of trimers (triabodies or tribodies). Tetrabodies have also been produced. They exhibit an even higher affinity to their targets than diabodies.

One embodiment provides a monoclonal antibody obtained from a substantially homogeneous population of antibodies, i.e., the individual antibodies within the population are identical except for possible naturally occurring mutations that may be present in a small subset of the antibody molecules. Monoclonal antibodies include “chimeric” antibodies in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, as long as they exhibit the desired antagonistic activity.

One embodiment provides antibodies and antigen binding fragments thereof the specifically bind to human KLRG1.

One embodiment provides antibodies produced by a hybridoma from the group consisting of K1A6, K1B9, K1F7, K2H11, K3G4, K3H11, K4C10, K4F4, K4F5, K7C12, K8F2, K9H1, or K10D5.

a. Chimeric and Humanized Antibodies

Another embodiment provides chimeric antibodies and antigen binding fragments thereof the specifically bind to human KLRG1.

Methods for producing chimeric antibodies are known in the art. See e.g., Morrison, Science, 229:1202 (1985); Oi et al., BioTechniques, 4:214 (1986); Gillies et al., J Immunol Methods, 125:191-202 (1989); and U.S. Pat. Nos. 6,311,415, 5,807,715, 4,816,567, and 4,816,397. Chimeric antibodies including one or more CDRs from a non-human species and framework regions from a human immunoglobulin molecule can be produced using a variety of techniques known in the art including, for example, CDR-grafting (EP 239,400; International Publication No. WO 91/09967; and U.S. Pat. Nos. 5,225,539, 5,530,101, and 5,585,089), veneering or resurfacing (EP 592,106; EP 519,596; Padlan, Molecular Immunology, 28(4/5):489-498 (1991); Studnicka et al., Protein Engineering, 7:805 (1994); and Roguska et al., Proc Natl Acad Sci USA, 91:969 (1994), and chain shuffling (U.S. Pat. No. 5,565,332).

The disclosed molecules can be human or humanized antibodies, or antigen binding fragments thereof. Many non-human antibodies (e.g., those derived from mice, rats, or rabbits) are naturally antigenic in humans, and thus can give rise to undesirable immune responses when administered to humans. Therefore, the use of human or humanized antibodies in the methods serves to lessen the chance that an antibody administered to a human will evoke an undesirable immune response.

Transgenic animals (e.g., mice) that are capable, upon immunization, of producing a full repertoire of human antibodies in the absence of endogenous immunoglobulin production can be employed. For example, it has been described that the homozygous deletion of the antibody heavy chain joining region (J(H)) gene in chimeric and germ-line mutant mice results in complete inhibition of endogenous antibody production. Transfer of the human germ-line immunoglobulin gene array in such germ-line mutant mice will result in the production of human antibodies upon antigen challenge.

Optionally, the antibodies are generated in other species and “humanized” for administration in humans. Humanized forms of non-human (e.g., murine) antibodies are chimeric immunoglobulins, immunoglobulin chains or fragments thereof (such as Fv, Fab, Fab′, F(ab′)₂, or other antigen-binding subsequences of antibodies) which contain minimal sequence derived from non-human immunoglobulin. Humanized antibodies include human immunoglobulins (recipient antibody) in which residues from a complementarity determining region (CDR) of the recipient antibody are replaced by residues from a CDR of a non-human species (donor antibody) such as mouse, rat or rabbit having the desired specificity, affinity and capacity. In some instances, Fv framework residues of the human immunoglobulin are replaced by corresponding non-human residues. Humanized antibodies may also contain residues that are found neither in the recipient antibody nor in the imported CDR or framework sequences. In general, the humanized antibody will contain substantially all of at least one, and typically two, variable domains, in which all or substantially all of the CDR regions correspond to those of a non-human immunoglobulin and all or substantially all of the FR regions are those of a human immunoglobulin consensus sequence. The humanized antibody optimally also will contain at least a portion of an immunoglobulin constant region (Fc), typically that of a human immunoglobulin.

Methods for humanizing non-human antibodies are well known in the art, see, for example, European Patent Nos. EP 239,400, EP 592,106, and EP 519,596; International Publication Nos. WO 91/09967 and WO 93/17105; U.S. Pat. Nos. 5,225,539, 5,530,101, 5,565,332, 5,585,089, 5,766,886, and 6,407,213; and Padlan, Molecular Immunology, 28(4/5):489-498 (1991); Studnicka et al., Protein Engineering 7(6):805-814 (1994); Roguska et al., PNAS, 91:969-973 (1994); Tan et al., J. Immunol. 169:1119-1125 (2002); Caldas et al., Protein Eng, 13:353-360 (2000); Morea et al., Methods, 20:267-79 (2000); Baca et al., J Biol Chem, 272:10678-10684 (1997); Roguska et al., Protein Eng, 9:895-904 (1996); Couto et al., Cancer Res, 55 (23 Supp):5973s-5977s (1995); Couto et al., Cancer Res, 55:1717-22 (1995); Sandhu, Gene, 150:409-10 (1994); Pedersen et al., J Mol Biol, 235:959-973 (1994); Jones et al., Nature, 321:522-525 (1986); Reichmann et al., Nature, 332:323-329 (1988); and Presta, Curr Op Struct Biol, 2:593-596 (1992)).

Generally, a humanized antibody has one or more amino acid residues introduced into it from a source that is non-human. These non-human amino acid residues are often referred to as “import” residues, which are typically taken from an “import” variable domain. Antibody humanization techniques generally involve the use of recombinant DNA technology to manipulate the DNA sequence encoding one or more polypeptide chains of an antibody molecule. Humanization can be essentially performed by substituting rodent CDRs or CDR sequences for the corresponding sequences of a human antibody. Accordingly, a humanized form of a non-human antibody (or a fragment thereof) is a chimeric antibody or fragment, wherein substantially less than an intact human variable domain has been substituted by the corresponding sequence from a non-human species. In practice, humanized antibodies are typically human antibodies in which some CDR residues and possibly some FR residues are substituted by residues from analogous sites in rodent antibodies.

The choice of human variable domains, both light and heavy, to be used in making the humanized antibodies can be very important in order to reduce antigenicity. According to the “best-fit” method, the sequence of the variable domain of a rodent antibody is screened against the entire library of known human variable domain sequences. The human sequence which is closest to that of the rodent is then accepted as the human framework (FR) for the humanized antibody. Another method uses a particular framework derived from the consensus sequence of all human antibodies of a particular subgroup of light or heavy chains. The same framework may be used for several different humanized antibodies.

It is further important that antibodies be humanized with retention of high affinity for the antigen and other favorable biological properties. To achieve this goal, humanized antibodies can be prepared by a process of analysis of the parental sequences and various conceptual humanized products using three dimensional models of the parental and humanized sequences. Three dimensional immunoglobulin models are commonly available and are familiar to those skilled in the art. Computer programs are available which illustrate and display probable three-dimensional conformational structures of selected candidate immunoglobulin sequences. Inspection of these displays permits analysis of the likely role of the residues in the functioning of the candidate immunoglobulin sequence, i.e., the analysis of residues that influence the ability of the candidate immunoglobulin to bind its antigen. In this way, FR residues can be selected and combined from the consensus and import sequence so that the desired antibody characteristic, such as increased affinity for the target antigen(s), is achieved. In general, the CDR residues are directly and most substantially involved in influencing antigen binding.

A human, humanized or chimeric antibody derivative can include substantially all of at least one, and typically two, variable domains in which all or substantially all of the CDR regions correspond to those of a non-human immunoglobulin (i.e., donor antibody) and all or substantially all of the framework regions are those of a human immunoglobulin consensus sequence. Such antibodies can also include at least a portion of an immunoglobulin constant region (Fc), typically that of a human immunoglobulin. The constant domains of such antibodies can be selected with respect to the proposed function of the antibody, in particular the effector function which may be required. In some embodiments, the constant domains of such antibodies are or can include human IgA, IgD, IgE, IgG or IgM domains. In a specific embodiment, human IgG constant domains, especially of the IgG1 and IgG3 isotypes are used, when the humanized antibody derivative is intended for a therapeutic use and antibody effector functions such as antibody-dependent cell-mediated cytotoxicity (ADCC) and complement-dependent cytotoxicity (CDC) activity are needed. In alternative embodiments, IgG2 and IgG4 isotypes are used when the antibody is intended for therapeutic purposes and antibody effector function is not required. Fc constant domains including one or more amino acid modifications which alter antibody effector functions such as those disclosed in U.S. Patent Application Publication Nos. 2005/0037000 and 2005/0064514.

The framework and CDR regions of a humanized antibody need not correspond precisely to the parental sequences, e.g., the donor CDR or the consensus framework can be mutagenized by substitution, insertion or deletion of at least one residue so that the CDR or framework residue at that site does not correspond to either the consensus or the donor antibody. In some embodiments, such mutations are not extensive. Usually, at least 75% of the humanized antibody residues will correspond to those of the parental framework region (FR) and CDR sequences, more often 90%, or greater than 95%. Humanized antibodies can be produced using variety of techniques known in the art, including, but not limited to, CDR-grafting (European Patent No. EP 239,400; International Publication No. WO 91/09967; and U.S. Pat. Nos. 5,225,539, 5,530,101, and 5,585,089), veneering or resurfacing (European Patent Nos. EP 592,106 and EP 519,596; Padlan, Molecular Immunology, 28(4/5):489-498 (1991); Studnicka et al., Protein Engineering, 7(6):805-814 (1994); and Roguska et al., Proc Natl Acad Sci 91:969-973 (1994), chain shuffling (U.S. Pat. No. 5,565,332), and techniques disclosed in, e.g., U.S. Pat. Nos. 6,407,213, 5,766,886, 5,585,089, International Publication No. WO 9317105, Tan et al., J Immunol, 169:1119-25 (2002), Caldas et al., Protein Eng, 13:353-60 (2000), Morea et al., Methods, 20:267-79 (2000), Baca et al., J Biol Chem, 272:10678-84 (1997), Roguska et al., Protein Eng, 9:895-904 (1996), Couto et al., Cancer Res, 55 (23 Supp):5973s-5977s (1995), Couto et al., Cancer Res, 55:1717-22 (1995), Sandhu, Gene, 150:409-10 (1994), Pedersen et al., J Mol Biol, 235:959-73 (1994), Jones et al., Nature, 321:522-525 (1986), Riechmann et al., Nature, 332:323 (1988), and Presta, Curr Op Struct Biol 2:593-596 (1992).

Often, framework residues in the framework regions will be substituted with the corresponding residue from the CDR donor antibody to alter, for example improve, antigen binding. These framework substitutions are identified by methods well known in the art, e.g., by modeling of the interactions of the CDR and framework residues to identify framework residues important for antigen binding and sequence comparison to identify unusual framework residues at particular positions. (See, e.g., Queen et al., U.S. Pat. No. 5,585,089; U.S. Publication Nos. 2004/0049014 and 2003/0229208; U.S. Pat. Nos. 6,350,861; 6,180,370; 5,693,762; 5,693,761; 5,585,089; and 5,530,101 and Riechmann et al., Nature 332:323 (1988)).

Human, chimeric or humanized derivatives of the disclosed murine anti-human KLRG1 antibodies can be used for in vivo methods in humans. Murine antibodies or antibodies of other species can be advantageously employed for many uses (for example, in vitro or in situ detection assays, acute in vivo use, etc.). Such a human or humanized antibody can include amino acid residue substitutions, deletions or additions in one or more non-human CDRs. The humanized antibody derivative can have substantially the same binding, stronger binding or weaker binding when compared to a non-derivative humanized antibody. In specific embodiments, one, two, three, four, or five amino acid residues of the CDR have been substituted, deleted or added (i.e., mutated). Completely human antibodies are particularly desirable for therapeutic treatment of human subjects.

Such human antibodies can be made by a variety of methods known in the art including phage display methods using antibody libraries derived from human immunoglobulin sequences (see U.S. Pat. Nos. 4,444,887 and 4,716,111; and International Publication Nos. WO 98/46645, WO 98/50433, WO 98/24893, WO 98/16654, WO 96/34096, WO 96/33735, and WO 91/10741). Such human antibodies can be produced using transgenic mice which are incapable of expressing functional endogenous immunoglobulins, but which can express human immunoglobulin genes.

For example, the human heavy and light chain immunoglobulin gene complexes can be introduced randomly or by homologous recombination into mouse embryonic stem cells. Alternatively, the human variable region, constant region, and diversity region can be introduced into mouse embryonic stem cells in addition to the human heavy and light chain genes. The mouse heavy and light chain immunoglobulin genes can be rendered non-functional separately or simultaneously with the introduction of human immunoglobulin loci by homologous recombination. In particular, homozygous deletion of the J_{H} region prevents endogenous antibody production. The modified embryonic stem cells are expanded and microinjected into blastocysts to produce chimeric mice. The chimeric mice are then bred to produce homozygous offspring which express human antibodies. The transgenic mice are immunized using conventional methodologies with a selected antigen, e.g., all or a portion of a polypeptide. Monoclonal antibodies directed against the antigen can be obtained from the immunized, transgenic mice using conventional hybridoma technology (see, e.g., U.S. Pat. No. 5,916,771). The human immunoglobulin transgenes harbored by the transgenic mice rearrange during B cell differentiation, and subsequently undergo class switching and somatic mutation. Thus, using such a technique, it is possible to produce therapeutically useful IgG, IgA, IgM and IgE antibodies. For an overview of this technology for producing human antibodies, see Lonberg et al., Int Rev Immunol, 13:65-93 (1995), which is incorporated herein by reference in its entirety). For a detailed discussion of this technology for producing human antibodies and human monoclonal antibodies and protocols for producing such antibodies, see, e.g., International Publication Nos. WO 98/24893, WO 96/34096, and WO 96/33735; and U.S. Pat. Nos. 5,413,923, 5,625,126, 5,633,425, 5,569,825, 5,661,016, 5,545,806, 5,814,318, and 5,939,598, which are incorporated by reference herein in their entirety. In addition, companies such as Abgenix, Inc. (Freemont, Calif.) and Medarex (Princeton, N.J.) can be engaged to provide human antibodies directed against a selected antigen using technology similar to that described above.

DNA sequences coding for human acceptor framework sequences include but are not limited to FR segments from the human germline VH segment VH1-18 and JH6 and the human germline VL segment VK-A26 and JK4. In a specific embodiment, one or more of the CDRs are inserted within framework regions using routine recombinant DNA techniques. The framework regions can be naturally occurring or consensus framework regions, and human framework regions (see, e.g., Chothia et al., “Structural Determinants In The Sequences of Immunoglobulin Variable Domain,” J Mol Biol, 278: 457-479 (1998), for a listing of human framework regions).

b. Single-Chain Antibodies

The KLRG1-binding molecules can be single-chain antibodies. Methods for the production of single-chain antibodies are well known to those of skill in the art. A single chain antibody is created by fusing together the variable domains of the heavy and light chains using a short peptide linker, thereby reconstituting an antigen binding site on a single molecule. Single-chain antibody variable fragments (scFvs) in which the C-terminus of one variable domain is tethered to the N-terminus of the other variable domain via a 15 to 25 amino acid peptide or linker have been developed without significantly disrupting antigen binding or specificity of the binding. The linker is chosen to permit the heavy chain and light chain to bind together in their proper conformational orientation. These Fvs lack the constant regions (Fc) present in the heavy and light chains of the native antibody.

c. Monovalent Antibodies

One embodiment provides monovalent antibodies specific for KLRG1, preferably human KLRG1. In vitro methods are also suitable for preparing monovalent antibodies. Digestion of antibodies to produce fragments thereof, particularly, Fab fragments, can be accomplished using routine techniques known in the art. For instance, digestion can be performed using papain. Papain digestion of antibodies typically produces two identical antigen binding fragments, called Fab fragments, each with a single antigen binding site, and a residual Fc fragment. Pepsin treatment yields a fragment, called the F(ab′)₂ fragment, that has two antigen combining sites and is still capable of cross-linking antigen.

The Fab fragments produced in the antibody digestion also contain the constant domains of the light chain and the first constant domain of the heavy chain. Fab′ fragments differ from Fab fragments by the addition of a few residues at the carboxy terminus of the heavy chain domain including one or more cysteines from the antibody hinge region. The F(ab′)₂ fragment is a bivalent fragment comprising two Fab′ fragments linked by a disulfide bridge at the hinge region. Fab′-SH is the designation herein for Fab′ in which the cysteine residue(s) of the constant domains bear a free thiol group. Antibody fragments originally were produced as pairs of Fab′ fragments which have hinge cysteines between them. Other chemical couplings of antibody fragments are also known.

d. Conjugates or Fusions of Antibody Fragments

One embodiment provides conjugates or fusions of antibody fragments specific for KLRG1, preferably human KLRG1. The targeting function of the antibody can be used therapeutically by coupling the antibody or a fragment thereof with a therapeutic agent. Such coupling of the antibody or fragment (e.g., at least a portion of an immunoglobulin constant region (Fc)) with the therapeutic agent can be achieved by making an immunoconjugate or by making a fusion protein, comprising the antibody or antibody fragment and the therapeutic agent.

Such coupling of the antibody or fragment with the therapeutic agent can be achieved by making an immunoconjugate or by making a fusion protein, or by linking the antibody or fragment to a nucleic acid such as an siRNA, comprising the antibody or antibody fragment and the therapeutic agent.

In some embodiments, the antibody is modified to alter its half-life. In some embodiments, it is desirable to increase the half-life of the antibody so that it is present in the circulation or at the site of treatment for longer periods of time. For example, it may be desirable to maintain titers of the antibody in the circulation or in the location to be treated for extended periods of time. Antibodies can be engineered with Fc variants that extend half-life, e.g., using Xtend™ antibody half-life prolongation technology (Xencor, Monrovia, Calif.). In other embodiments, the half-life of the anti-DNA antibody is decreased to reduce potential side effects. The conjugates disclosed can be used for modifying a given biological response. The drug moiety is not to be construed as limited to classical chemical therapeutic agents. For example, the drug moiety may be a protein or polypeptide possessing a desired biological activity. Such proteins may include, for example, a toxin such as abrin, ricin A, pseudomonas exotoxin, or diphtheria toxin.

e. Mono and Multi-Specific Antibodies

In some embodiments the disclosed antibodies are monospecific, binding only to KLRG1, preferably human KLRG1. Bispecific derivatives of such antibodies, trispecific derivatives of such antibodies or derivative antibodies of greater multi-specificity, that exhibit specificity to different immune system targets in addition to their specificity for human KLRG1 are also provided. For example, such antibodies can bind to both human KLRG1 and to an antigen that is important for targeting the antibody to a particular cell type or tissue (for example, to an antigen associated with a cancer antigen of a tumor being treated). In another embodiment, such multispecific antibody binds to molecules (receptors or ligands) involved in alternative immunomodulatory pathways, such as B7-H1, PD-1, CTLA4, TIM3, OX40, CD40, GITR, 4-1-BB, LIGHT, LAG3 or CD71 in order to enhance the immunomodulatory effects and combine multiple mechanisms of action, such as ligand blocking, immune cell activation and direct tumor targeting, in one molecule. In particular, KLRG1 and CD71(transferrin receptor) bispecific antibodies may be developed due to the natural association and or colocalization of KLRG1 with CD71.

f. Derivatives

Production and use of “derivatives” of any of the disclosed KLRG1-binding molecules are also disclosed. A derivative molecule, for example an antibody or antibody fragment, can be modified by chemical modifications using techniques known to those of skill in the art, including, but not limited to, specific chemical cleavage, acetylation, formulation, metabolic synthesis of tunicamycin, etc. The term derivative encompasses non amino acid modifications, for example, amino acids that can be glycosylated (e.g., have altered mannose, 2-N-acetylglucosamine, galactose, fucose, glucose, sialic acid, 5-N-acetylneuraminic acid, 5-glycolneuraminic acid, etc. content), acetylated, pegylated, phosphorylated, amidated, derivatized by known protecting/blocking groups, proteolytic cleavage, linked to a cellular ligand or other protein, etc. In some embodiments, the altered carbohydrate modifications modulate one or more of the following: solubilization of the antibody, facilitation of subcellular transport and secretion of the antibody, promotion of antibody assembly, conformational integrity, and antibody-mediated effector function.

In a specific embodiment the altered carbohydrate modifications enhance antibody mediated effector function relative to the antibody lacking the carbohydrate modification. Carbohydrate modifications that lead to altered antibody mediated effector function are well known in the art (for example, see Shields, R. L. et al., “Lack of Fuctose on Human IgG N-Linked Oligosaccharide Improves Binding to Human Fcgamma RIII and Antibody-Dependent Cellular Toxicity,” J Biol Chem, 277(30): 26733-26740 (2002); Davies J. et al. “Expression of GnTIII in a Recombinant Anti-CD20 CHO Production Cell Line: Expression of Antibodies With Altered Glycoforms Leads to an Increase in ADCC Through Higher Affinity for FC Gamma RIII”, Biotechnology & Bioengineering, 74(4): 288-294 (2001). Methods of altering carbohydrate contents are known to those skilled in the art, see, e.g., Wallick, S. C. et al., “Glycosylation of a VH Residue of a Monoclonal Antibody Against Alpha (1-6) Dextran Increases Its Affinity For Antigen,” J Exp Med, 168(3): 1099-1109 (1988); Tao, M. H. et al., “Studies of Aglycosylated Chimeric Mouse-Human IgG. Role of Carbohydrate in the Structure and Effector Functions Mediated by the Human IgG Constant Region”, J Immunol, 143(8): 2595-2601(1989); Routledge, E. G. et al., “The Effect Of Aglycosylation on the Immunogenicity of a Humanized Therapeutic CD3 Monoclonal Antibody”, Transplantation, 60(8):847-53 (1995); Elliott, S. et al., “Enhancement of Therapeutic Protein In Vivo Activities Through Glycoengineering”, Nature Biotechnol, 21:414-21 (2003); Shields, R. L. et al., “Lack of Fucose on Human IgG N-Linked Oligosaccharide Improves Binding to Human Fcgamma RIII and Antibody-Dependent Cellular Toxicity”, J Biol Chem, 277(30): 26733-26740 (2002).

The disclosed antibodies can be modified by recombinant means to increase greater efficacy of the antibody in mediating the desired function. Thus, antibodies can be modified by substitutions using recombinant means. Typically, the substitutions will be conservative substitutions. For example, at least one amino acid in the constant region of the antibody can be replaced with a different residue. See, e.g., U.S. Pat. Nos. 5,624,821, 6,194,551, Application No. WO 9958572; and Angal, et al., Mol Immunol, 30:105-08 (1993). The modification in amino acids includes deletions, additions, substitutions of amino acids. In some cases, such changes are made to reduce undesired activities, e.g., complement-dependent cytotoxicity. Frequently, the antibodies are labeled by joining, either covalently or non-covalently, a substance which provides for a detectable signal. A wide variety of labels and conjugation techniques are known and are reported extensively in both the scientific and patent literature. These antibodies can be screened for binding to KLRG1 polypeptides, or fragments, or fusions thereof. See e.g., Antibody Engineering: A Practical Approach (Oxford University Press, 1996).

In some embodiments, an antibody derivative will possess a similar or identical function as the parental antibody. In another embodiment, an antibody derivative will exhibit an altered activity relative to the parental antibody. For example, a derivative antibody (or fragment thereof) can bind to its epitope more tightly or be more resistant to proteolysis than the parental antibody.

Substitutions, additions or deletions in the derivatized antibodies can be in the Fc region of the antibody and can thereby serve to modify the binding affinity of the antibody to one or more FcγR. Methods for modifying antibodies with modified binding to one or more FcγR are known in the art, see, e.g., PCT Publication Nos. WO 04/029207, WO 04/029092, WO 04/028564, WO 99/58572, WO 99/51642, WO 98/23289, WO 89/07142, WO 88/07089, and U.S. Pat. Nos. 5,843,597 and 5,642,821.

In some embodiments, antibodies whose Fc region have been deleted (for example, an Fab or F(ab)2, etc.) or modified so that the molecule exhibits diminished or no Fc receptor (FcR) binding activity, or exhibits enhanced antibody-dependent cell-mediated cytotoxicity (ADCC) or complement dependent cytotoxicity (CDC) activities. In some embodiments, the antibodies have altered affinity for an activating FcγR, e.g., FcγRIIIA. Such modifications can also have an altered Fc-mediated effector function. Modifications that affect Fc-mediated effector function are well known in the art (see U.S. Pat. No. 6,194,551, and WO 00/42072). In one particular embodiment, the modification of the Fc region results in an antibody with an altered antibody-mediated effector function, an altered binding to other Fc receptors (e.g., Fc activation receptors), an altered antibody-dependent cell-mediated cytotoxicity (ADCC) activity, an altered C1q binding activity, an altered complement-dependent cytotoxicity activity (CDC), a phagocytic activity, or any combination thereof.

Derivatized antibodies can be used to alter the half-lives (e.g., serum half-lives) of parental antibodies in a mammal, such as a human. For example, such alteration can result in a half-life of greater than 15 days, greater than 20 days, greater than 25 days, greater than 30 days, greater than 35 days, greater than 40 days, greater than 45 days, greater than 2 months, greater than 3 months, greater than 4 months, or greater than 5 months. The increased half-lives of the humanized antibodies or fragments thereof in a mammal, such as a human, results in a higher serum titer of said antibodies or antibody fragments in the mammal, and thus, reduces the frequency of the administration of said antibodies or antibody fragments and/or reduces the concentration of said antibodies or antibody fragments to be administered. Antibodies or fragments thereof having increased in vivo half-lives can be generated by techniques known to those of skill in the art. For example, antibodies or fragments thereof with increased in vivo half-lives can be generated by modifying (e.g., substituting, deleting or adding) amino acid residues identified as involved in the interaction between the Fc domain and the FcRn receptor. The humanized antibodies can be engineered to increase biological half-lives (see, e.g. U.S. Pat. No. 6,277,375). For example, humanized antibodies can be engineered in the Fc-hinge domain to have increased in vivo or serum half-lives.

Antibodies or fragments thereof with increased in vivo half-lives can be generated by attaching to said antibodies or antibody fragments polymer molecules such as high molecular weight polyethyleneglycol (PEG). PEG can be attached to said antibodies or antibody fragments with or without a multifunctional linker either through site-specific conjugation of the PEG to the N- or C-terminus of said antibodies or antibody fragments or via epsilon-amino groups present on lysine residues. Linear or branched polymer derivatization that results in minimal loss of biological activity will be used. The degree of conjugation will be closely monitored by SDS-PAGE and mass spectrometry to ensure proper conjugation of PEG molecules to the antibodies. Unreacted PEG can be separated from antibody-PEG conjugates by, e.g., size exclusion or ion-exchange chromatography.

The antibodies can also be modified by the methods and coupling agents described by Davis et al. (See U.S. Pat. No. 4,179,337) in order to provide compositions that can be injected into the mammalian circulatory system with substantially no immunogenic response.

The framework residues of the humanized antibodies can be modified. Residues in the framework regions can be substituted with the corresponding residue from the CDR donor antibody to alter, for example improve, antigen binding. These framework substitutions can be identified by methods well known in the art, e.g., by modeling of the interactions of the CDR and framework residues to identify framework residues important for antigen binding and sequence comparison to identify unusual framework residues at particular positions. (See, e.g., U.S. Pat. No. 5,585,089; and Riechmann, L. et al., “Reshaping Human Antibodies For Therapy,” Nature, 332:323-327 (1988).

The disclosed KLRG1-binding molecules can be recombinantly fused or chemically conjugated (including both covalently and non-covalently conjugations) to a heterologous molecule (i.e., an unrelated molecule). The fusion does not necessarily need to be direct, but may occur through linker sequences.

In some embodiments such heterologous molecules are polypeptides having at least 10, at least 20, at least 30, at least 40, at least 50, at least 60, at least 70, at least 80, at least 90 or at least 100 amino acids. Such heterologous molecules can alternatively be enzymes, hormones, cell surface receptors, drug moieties, such as: macrophage-specific targeting reagents (such as the intracellular carboxylesterase, hCE1 (Needham, L. A. et al., “Drug Targeting To Monocytes and Macrophages Using Esterase-Sensitive Chemical Motif,” J Pharmacol Exp Ther, DOI:10.1124/jpet.111.183640, Chitin and Chitosan, (2011); (Muzzarelli, R. A., “Chitins and Chitosans as Immunoadjuvants and Non-Allergenic Drug Carriers”, Mar Drugs, 8(2):292-312 (2010), galactosylated low-density lipoprotein (Wu, F. et al., “Galactosylated LDL Nanoparticles: A Novel Targeting Delivery System to Deliver Antigen to Macrophages and Enhance Antigen Specific T Cell Responses”, Molec Pharm, 6(5):1506-1517 (2009), N-formyl-Met-Leu-Phe (fMLF), a macrophage-specific chemo-attractant (Wan, L. et al., “Optimizing Size and Copy Number for PEG-Fmlf (N-Formyl-Methionyl-Leucyl-Phenylalanine) Nanocarrier Uptake by Macrophages,” Bioconjug Chem, 19(1):28-38 (2008), maleylated or mannosylated protein, such as maleylated albumin (Anatelli, F. et al., “Macrophage-Targeted Photosensitizer Conjugate Delivered by Intratumoral Injection,” Mol Pharm, 3(6):654-664 (2006); Bansal, P. et al., “MHC Class I-Restricted Presentation of Maleylated Protein Binding to Scavenger Receptors”, J Immunol, 162(8):4430-4437) (1999); see also Mukhopadhyay, A. et al., “Intracellular Delivery of Drugs to Macrophages”, Adv Biochem Eng Biotechnol, 84:183-209 (2003)), toxins (such as abrin, ricin A, pseudomonas exotoxin (i.e., PE-40), diphtheria toxin, ricin, gelonin, or pokeweed antiviral protein), proteins (such as tumor necrosis factor, interferon (e.g., α-interferon, β-interferon), nerve growth factor, platelet derived growth factor, tissue plasminogen activator, or an apoptotic agent (e.g., tumor necrosis factor-α, tumor necrosis factor-β)), biological response modifiers (such as, for example, a lymphokine (e.g., interleukin-1 (“IL-1”), interleukin-2 (“IL-2”), interleukin-6 (“IL-6”)), granulocyte macrophage colony stimulating factor (“GM-CSF”), granulocyte colony stimulating factor (“G-CSF”), or macrophage colony stimulating factor, (“M-CSF”), or growth factors (e.g., growth hormone (“GH”))), cytotoxins (e.g., a cytostatic or cytocidal agent, such as paclitaxol, cytochalasin B, gramicidin D, ethidium bromide, emetine, mitomycin, etoposide, tenoposide, vincristine, vinblastine, colchicin, doxorubicin, daunorubicin, dihydroxy anthracin dione, mitoxantrone, mithramycin, actinomycin D, 1-dehydrotestosterone, glucocorticoids, procaine, tetracaine, lidocaine, propranolol, and puromycin and analogs or homologs thereof), antimetabolites (e.g., methotrexate, 6-mercaptopurine, 6-thioguanine, cytarabine, 5-fluorouracil decarbazine), alkylating agents (e.g., mechlorethamine, thioepa chlorambucil, melphalan, BiCNU® (carmustine; BSNU) and lomustine (CCNU), cyclothosphamide, busulfan, dibromomannitol, streptozotocin, mitomycin C, and cisdichlorodiamine platinum (II) (DDP) cisplatin), anthracyclines (e.g., daunorubicin (formerly daunomycin) and doxorubicin), antibiotics (e.g., dactinomycin (formerly actinomycin), bleomycin, mithramycin, and anthramycin (AMC)), or anti-mitotic agents (e.g., vincristine and vinblastine).

In another embodiment, the molecules are conjugated to a second antibody to form an antibody heteroconjugate as described by Segal in U.S. Pat. No. 4,676,980. Such heteroconjugate antibodies can additionally bind to haptens (such as fluorescein, etc.), or to cellular markers (e.g., 4-1-BB, B7-H1, PD-1, CD4, CD8, CD14, CD25, CD27, CD40, CD68, CD71, CD163, CTLA4, GITR, LAG-3, OX40, TIM3, TIM4, TLR2, LIGHT, etc.) or to cytokines (e.g., IL-4, IL-7, IL-10, IL-12, IL-15, IL-17, TGF-beta, IFNg, Flt3, BLys) or chemokines (e.g., CCL21), etc.

The Fc portion of the fusion protein can be varied by isotype or subclass, can be a chimeric or hybrid, and/or can be modified, for example to improve effector functions, control of half-life, tissue accessibility, augment biophysical characteristics such as stability, and improve efficiency of production (and less costly). Many modifications useful in construction of disclosed fusion proteins and methods for making them are known in the art, see for example Mueller, J. P. et al., “Humanized Porcine VCAM-Specific Monoclonal Antibodies with Chimeric IgG2/G4 Constant Regions Block Human Leukocyte Binding to Porcine Endothelial Cells,” Mol Immun, 34(6):441-452 (1997), Swann, P. G., “Considerations for the Development of Therapeutic Monoclonal Antibodies”, Curr Opin Immun, 20:493-499 (2008), and Presta, L. G., “Molecular Engineering And Design Of Therapeutic Antibodies”, Curr Opin Immun, 20:460-470 (2008). In some embodiments the Fc region is the native IgG1, IgG2, or IgG4 Fc region. In some embodiments the Fc region is a hybrid, for example a chimeric consisting of IgG2/IgG4 Fc constant regions. Modifications to the Fc region include, but are not limited to, IgG4 modified to prevent binding to Fc gamma receptors and complement, IgG1 modified to improve binding to one or more Fc gamma receptors, IgG1 modified to minimize effector function (amino acid changes), IgG1 with altered/no glycan (typically by changing expression host), and IgG1 with altered pH-dependent binding to FcRn. The Fc region can include the entire hinge region, or less than the entire hinge region.

The therapeutic outcome in patients treated with rituximab (a chimeric mouse/human IgG1 monoclonal antibody against CD20) for non-Hodgkin's lymphoma or Waldenstrom's macroglobulinemia correlated with the individual's expression of allelic variants of Fcγ receptors with distinct intrinsic affinities for the Fc domain of human IgG1. In particular, patients with high affinity alleles of the low affinity activating Fc receptor CD16A (FcγRIIIA) showed higher response rates and, in the cases of non-Hodgkin's lymphoma, improved progression-free survival. Therefore, the Fc domain can the disclosed antibodies and fragments contain one or more amino acid insertions, deletions or substitutions that reduce binding to the low affinity inhibitory Fc receptor CD32B (FcγRIIB) and retain wild-type levels of binding to or enhance binding to the low affinity activating Fc receptor CD16A (FcγRIIIA).

Another embodiment includes IgG2-4 hybrids and IgG4 mutants that have reduced binding to FcγR, which increases their half-life. Representative IgG2-4 hybrids and IgG4 mutants are described in Angal, S. et al., “A Single Amino Acid Substitution Abolishes the Heterogeneity of Chimeric Mouse/Human (Igg4) Antibody”, Molec Immunol, 30(1):105-108 (1993); Mueller, J. P. et al., “Humanized Porcine VCAM-Specific Monoclonal Antibodies with Chimeric IgG2/G4 Constant Regions Block Human Leukocyte Binding to Porcine Endothelial Cells”, Mol Immun, 34(6):441-452 (1997); and U.S. Pat. No. 6,982,323. In some embodiments the IgG1 and/or IgG2 domain is modified; for example, Angal, S. et al. (1993) describe IgG1 and IgG2 variants in which serine 241 is replaced with proline.

In some embodiments, the Fc domain of such molecules contains amino acid insertions, deletions or substitutions that enhance binding to CD16A. A large number of substitutions in the Fc domain of human IgG1 that increase binding to CD16A and reduce binding to CD32B are known in the art and are described in Stavenhagen, J. B. et al., “Fc Optimization of Therapeutic Antibodies Enhances Their Ability to Kill Tumor Cells In Vitro And Controls Tumor Expansion In Vivo Via Low-Affinity Activating Fcgamma Receptors”, Cancer Res, 57(18):8882-8890 (2007). Exemplary variants of human IgG1 Fc domains with reduced binding to CD32B and/or increased binding to CD16A contain F243L, R929P, Y300L, V305I or P296L substitutions. These amino acid substitutions can be present in a human IgG1 Fc domain in any combination. In one embodiment, the human IgG1 Fc domain variant contains a F243L, R929P and Y300L substitution. In another embodiment, the human IgG1 Fc domain variant contains a F243L, R929P, Y300L, V305I and P296L substitution. In another embodiment, the human IgG1 Fc domain variant contains an N297Q substitution, as this mutation abolishes FcR binding.

Techniques for conjugating therapeutic moieties to antibodies are well known; see, e.g., Arnon et al., “Monoclonal Antibodies For Immunotargeting of Drugs In Cancer Therapy”, in Monoclonal Antibodies and Cancer Therapy, Reisfeld et al. (Eds.), pp. 243-56, Alan R. Liss, Inc. (1985)); Hellstrom et al., “Antibodies For Drug Delivery”, in Controlled Drug Delivery (2nd Ed.), Robinson et al. (Eds.), pp. 623-53, Marcel Dekker, Inc. (1987)); Thorpe, “Antibody Carriers of Cytotoxic Agents in Cancer Therapy: A Review”, in Monoclonal Antibodies '84: Biological And Clinical Applications, Pinchera et al. (Eds.), pp. 475-506 (1985)); “Analysis, Results, And Future Prospective of the Therapeutic Use of Radiolabeled Antibody in Cancer Therapy”, in Monoclonal Antibodies For Cancer Detection And Therapy, Baldwin et al. (Eds.), pp. 303-16 (1985), Academic Press; and Thorpe et al., “The Preparation And Cytotoxic Properties Of Antibody-Toxin Conjugates”, Immunol Rev, 62:119-158 (1982).

Any of the disclosed molecules can be fused to marker sequences, such as a peptide, to facilitate purification. In some embodiments, the marker amino acid sequence is a hexa-histidine peptide, the hemagglutinin “HA” tag, which corresponds to an epitope derived from the influenza hemagglutinin protein (Wilson, I. A. et al., “The Structure Of An Antigenic Determinant In A Protein,” Cell, 37:767-778 (1984)) and the “flag” tag (Knappik, A. et al., “An Improved Affinity Tag Based on the FLAG Peptide for the Detection and Purification of Recombinant Antibody Fragments,” Biotechniques, 17(4):754-761 (1994)).

The disclosed KLRG1-binding molecules can be conjugated to a diagnostic or therapeutic agent, or another molecule for which serum half-life is desired to be increased. The antibodies can be used diagnostically (in vivo, in situ or in vitro) to, for example, monitor the development or progression of a disease, disorder or infection as part of a clinical testing procedure to, e.g., determine the efficacy of a given treatment regimen or to select patients more likely to respond to a particular therapy (such as those expressing high levels of KLRG1).

Detection can be facilitated by coupling the molecule, such as antibody or an antigen binding fragment thereof, to a detectable substance. Examples of detectable substances include various enzymes, prosthetic groups, fluorescent materials, luminescent materials, bioluminescent materials, radioactive materials, positron emitting metals, and nonradioactive paramagnetic metal ions. The detectable substance can be coupled or conjugated either directly to the antibody or indirectly, through an intermediate (such as, for example, a linker known in the art) using techniques known in the art. See, for example, U.S. Pat. No. 4,741,900 for metal ions which can be conjugated to antibodies for use as diagnostics. Such diagnosis and detection can be accomplished by coupling the antibody to detectable substances including, but not limited to, various enzymes, enzymes including, but not limited to, horseradish peroxidase, alkaline phosphatase, beta-galactosidase, or acetylcholinesterase; prosthetic group complexes such as, but not limited to, streptavidin/biotin and avidin/biotin; fluorescent materials such as, but not limited to, umbelliferone, fluorescein, fluorescein isothiocyanate, rhodamine, dichlorotriazinylamine fluorescein, dansyl chloride or phycoerythrin; luminescent material such as, but not limited to, luminol; bioluminescent materials such as, but not limited to, luciferase, luciferin, and aequorin; radioactive material such as, but not limited to, bismuth (²¹³Bi), carbon (¹⁴C), chromium (⁵¹Cr), cobalt (⁵⁷Co), fluorine (¹⁸F), gadolinium (¹⁵³Gd, ¹⁵⁹Gd), gallium (⁶⁸Ga, ⁶⁷Ga), germanium (⁶⁸Ge), holmium (¹⁶⁶Ho), indium (¹¹⁵In, ¹¹³In, ¹¹²In, ¹¹¹In), iodine (¹³¹I, ¹²⁵I, ¹²³I, ¹²¹I), lanthanum (¹⁴⁰La), lutetium (¹⁷⁷Lu), manganese (⁵⁴Mn), molybdenum (⁹⁹Mo), palladium (¹⁰³Pd), phosphorous (³²P), praseodymium (¹⁴²Pr), promethium (¹⁴⁹Pm), rhenium (¹⁸⁶Re, ¹⁸⁸Re), rhodium (¹⁰⁵Rh), ruthenium (⁹⁷Ru), samarium (¹⁵³Sm), scandium (⁴⁷Sc), selenium (⁷⁵Se), strontium (⁸⁵Sr), sulfur (³⁵S), technetium (⁹⁹Tc), thallium (²⁰¹Ti), tin (¹¹³Sn, ¹¹⁷Sn), tritium (³H), xenon (¹³³Xe), ytterbium (¹⁶⁹Yb, ¹⁷⁵Yb), yttrium (⁹⁰Y), zinc (⁶⁵Zn); positron emitting metals using various positron emission tomographies, and nonradioactive paramagnetic metal ions.

The disclosed molecules can be attached to solid supports, which are particularly useful for immunoassays or purification of the target antigen or of other molecules that are capable of binding to target antigen that has been immobilized to the support via binding to an antibody or antigen-binding fragment. Such solid supports include, but are not limited to, glass, cellulose, polyacrylamide, nylon, polystyrene, polyvinyl chloride or polypropylene.

Nucleic acid molecules (DNA or RNA) that encode any such antibodies, fusion proteins or fragments, as well as vector molecules (such as plasmids) that are capable of transmitting or of replicating such nucleic acid molecules are also disclosed. The nucleic acids can be single-stranded, double-stranded, can contain both single-stranded and double-stranded portions.

3. Method of Making

The KLRG1-binding molecules can be produced by any method known in the art useful for the production of polypeptides, e.g., in vitro synthesis, recombinant DNA production, and the like. The humanized antibodies are typically produced by recombinant DNA technology. The antibodies can be produced using recombinant immunoglobulin expression technology. The recombinant production of immunoglobulin molecules, including humanized antibodies are described in U.S. Pat. No. 4,816,397 (Boss et al.), U.S. Pat. Nos. 6,331,415 and 4,816,567 (both to Cabilly et al.), U.K. patent GB 2,188,638 (Winter et al.), and U.K. patent GB 2,209,757. Techniques for the recombinant expression of immunoglobulins, including humanized immunoglobulins, can also be found, in Goeddel et al., “Gene Expression Technology Methods in Enzymology”, Vol. 185 Academic Press, (1991), and Borreback, “Antibody Engineering”, W. H. Freeman (1992). Additional information concerning the generation, design and expression of recombinant antibodies can be found in Mayforth, “Designing Antibodies”, Academic Press, San Diego (1993).

An exemplary process for the production of the recombinant chimeric antibodies can include the following: a) constructing, by conventional molecular biology methods, an expression vector that encodes and expresses an antibody heavy chain in which the CDRs and variable region of an anti-KLRG1 antibody are fused to an Fc region derived from a human immunoglobulin, thereby producing a vector for the expression of a chimeric antibody heavy chain; b) constructing, by conventional molecular biology methods, an expression vector that encodes and expresses an antibody light chain of the murine anti-human KLRG1 monoclonal antibody, thereby producing a vector for the expression of chimeric antibody light chain; c) transferring the expression vectors to a host cell by conventional molecular biology methods to produce a transfected host cell for the expression of chimeric antibodies; and d) culturing the transfected cell by conventional cell culture techniques so as to produce chimeric antibodies.

An exemplary process for the production of the recombinant humanized antibodies can include the following: a) constructing, by conventional molecular biology methods, an expression vector that encodes and expresses an anti-human KLRG1 heavy chain in which the CDRs and a minimal portion of the variable region framework that are required to retain donor antibody binding specificity are derived from the humanized variants of anti-human KLRG1 antibody(ies), and the remainder of the antibody is derived from a human immunoglobulin, thereby producing a vector for the expression of a humanized antibody heavy chain; b) constructing, by conventional molecular biology methods, an expression vector that encodes and expresses an antibody light chain in which the CDRs and a minimal portion of the variable region framework that are required to retain donor antibody binding specificity are derived from a non-human immunoglobulin, such as the disclosed murine anti-human KLRG1 antibodies, and the remainder of the antibody is derived from a human immunoglobulin, thereby producing a vector for the expression of humanized antibody light chain; c) transferring the expression vectors to a host cell by conventional molecular biology methods to produce a transfected host cell for the expression of humanized antibodies; and d) culturing the transfected cell by conventional cell culture techniques so as to produce humanized antibodies.

With respect to either exemplary method, host cells can be co-transfected with such expression vectors, which can contain different selectable markers but, with the exception of the heavy and light chain coding sequences, can be identical. This procedure provides for equal expression of heavy and light chain polypeptides. Alternatively, a single vector can be used which encodes both heavy and light chain polypeptides. The coding sequences for the heavy and light chains can include cDNA or genomic DNA or both. The host cell used to express the recombinant antibody can be either a bacterial cell such as Escherichia coli, or a eukaryotic cell (e.g., a Chinese hamster ovary (CHO) cell or a HEK-293 cell). The choice of expression vector is dependent upon the choice of host cell, and can be selected so as to have the desired expression and regulatory characteristics in the selected host cell. Other cell lines that can be used include, but are not limited to, CHO-K1, NSO, and PER.C6® or HAT-sensitive myeloma cells (Li, Feng, et al., MAbs, 2(5): 466-477 (2010)).

Any of the disclosed antibodies can be used to generate anti-idiotype antibodies using techniques well known to those skilled in the art (see, e.g., Greenspan, N. S., et al., “Idiotypes: Structure And Immunogenicity”, FASEB J. 7:437-444 (1989); and Nisinoff, A., “Idiotypes: Concepts And Applications”, J Immunol, 147(8):2429-2438 (1991)).

In one embodiment, clones producing the disclosed antibodies can be screened using ELISA bound to a plate coated with KLRG1 Fc. The clones can be further screened using flow cytometry to detect binding of KLRG1 mAbs to cells transfected with full-length KLRG1. Following selection of KLRG1 binding mAb clones, the clones can be screened for blocking the interaction of KLRG1 with E-cadherin (and potentially N- and R-cadherins). To do so, cells transfected with either KLRG1 or E-cadherin can be co-incubated with KLRG1 mAbs and either soluble E-cadherin Fc or KLRG1 Fc, respectively. Cells are washed and stained with secondary mAb to assess blockade of KLRG1-E-cadherin interaction. To select for KLRG1 mAbs with selective function as either antagonists (blocking) or agonists (signal inducing), reporter cells lines can be developed to assess the presence or absence of KLRG1 signaling following mAb binding to KLRG1. In addition, functional in vitro studies with primary T cells will be used to investigate KLRG1 antagonist and agonist functionality of KLRG1 mAb clones.

KLRG1 mAbs generated against human KLRG1 protein as described above will also be tested for binding to murine and primate KLRG1. KLRG1 mAbs that are cross-reactive between human and murine species can be used for in vivo studies in mice. Additionally, both mouse and human mAb panels may be developed for functional studies due to the relatively low sequence identity between human and mouse KLRG1.

B. KLRG1 Ligand-Binding Molecules

Molecules that bind to KLRG1 ligands, such as KLRG1 proteins, KLRG1 fusion proteins, and fragments and variants thereof are also provided. The KLRG1 ligand-binding molecule can bind to a KLRG1 ligand such as cadherins, preferably E-cadherin. In some embodiments, the KLRG1 ligand-binding molecule blocks or otherwise reduces interaction between KLRG1 and its ligand, without inducing signal transduction through KLRG1 or its ligand. KLRG1 ligand-binding molecules can be used to modulate KLRG1 activity as discussed in more detail below, and can be used to therapeutically to treat a subject in need thereof.

1. KLRG1 Polypeptides

In some embodiments, the KLRG1 ligand-binding molecule is KLRG1, or fragment or variant thereof. For example, in some embodiments, the KLRG1 ligand-binding molecules includes a polypeptide at least 50, 60, 70, 80, 85, 90, 95, 96, 97, 98, 99, or 100% identical to SEQ ID NO:1 or 2, or a fragment thereof such as a subdomain thereof. In some embodiments, the KLRG1 polypeptide is soluble or otherwise cell-free. For example, in some embodiments, the KLRG1 ligand-binding molecule lacks one or more of the transmembrane domain, the cytoplasmic domain, or the leader sequence.

2. KLRG1 Fusion Proteins

In some embodiments, the KLRG1 ligand-binding molecule is a KLRG1 fusion protein. The fusion protein can be an agonist or antagonist KLRG1 fusion protein. Antagonist fusion proteins contain KLRG1 polypeptides, preferably the ECD of KLRG1, coupled to other polypeptides to form fusion proteins. Agonist fusion proteins contain KLRG1 ligand polypeptides, preferably the ECD of the ligand polypeptide, coupled to other polypeptides to form fusion proteins. Preferred ligands are E-Cadherin, N-Cadherin, R-Cadherin, or a combination thereof. KLRG1 fusion polypeptides can have a first fusion partner comprising all or a part of a KLRG1 protein or KLRG1 ligand fused (i) directly to a second polypeptide or, (ii) optionally, fused to a linker peptide sequence that is fused to the second polypeptide. The fusion proteins optionally contain a domain that functions to dimerize or multimerize two or more fusion proteins. In some embodiments the fusion protein is not or does not dimerize or multimerize. The peptide/polypeptide linker domain can either be a separate domain, or alternatively can be contained within one of one of the other domains (KLRG1 polypeptide, KLRG1 ligand polypeptide, or second polypeptide) of the fusion protein. Similarly, the domain that functions to dimerize or multimerize the fusion proteins can either be a separate domain, or alternatively can be contained within one of one of the other domains (KLRG1 polypeptide, KLRG1 ligand polypeptide, second polypeptide or peptide/polypeptide linker domain) of the fusion protein. In some embodiments, the dimerization/multimerization domain and the peptide/polypeptide linker domain are the same.

Fusion proteins disclosed herein are of formula I:

N—R₁—R₂—R₃—C

wherein “N” represents the N-terminus of the fusion protein, “C” represents the C-terminus of the fusion protein, “R₁” is a KLRG1 polypeptide or KLRG1 ligand polypeptide, “R₂” is an optional peptide/polypeptide linker domain, and “R₃” is a second polypeptide. Alternatively, R₃ may be the KLRG1 polypeptide or KLRG1 ligand polypeptide and R₁ may be the second polypeptide.

The fusion proteins can be dimerized or multimerized. Dimerization or multimerization can occur between or among two or more fusion proteins through dimerization or multimerization domains. Alternatively, dimerization or multimerization of fusion proteins can occur by chemical crosslinking. The dimers or multimers that are formed can be homodimeric/homomultimeric or heterodimeric/heteromultimeric. As discussed above, in some embodiments the fusion protein is not or does not dimerize or multimerize.

In some embodiments, the second polypeptide contains one or more domains of an immunoglobulin heavy chain constant region, for example an amino acid sequence corresponding to the hinge, C_{H}2 and/or C_{H}3 regions of a human immunoglobulin Cγ1 chain, the hinge, C_{H}2 and/or C_{H}3 regions of a murine immunoglobulin Cγ2a chain, C_{H}2 and/or C_{H}3 regions of a human immunoglobulin Cγ1, etc.

The Fc portion of the fusion protein may be varied by isotype or subclass, may be a chimeric or hybrid, and/or may be modified, for example to improve effector functions, control of half-life, tissue accessibility, augment biophysical characteristics such as stability, and improve efficiency of production (and less costly). Many modifications useful in construction of disclosed fusion proteins and methods for making them are known in the art, see for example Mueller, et al., Mol Immun, 34(6):441-452 (1997), Swann, et al., Cur Opin Immun, 20:493-499 (2008), and Presta, Cur Opin Immun, 20:460-470 (2008). In some embodiments the Fc region is the native IgG1, IgG2, or IgG4 Fc region. In some embodiments the Fc region is a hybrid, for example a chimeric consisting of IgG2/IgG4 Fc constant regions. Modifications to the Fc region include, but are not limited to, IgG4 modified to prevent binding to Fc gamma receptors and complement, IgG1 modified to improve binding to one or more Fc gamma receptors, IgG1 modified to minimize effector function (amino acid changes), IgG1 with altered/no glycan (typically by changing expression host), and IgG1 with altered pH-dependent binding to FcRn. The Fc region may include the entire hinge region, or less than the entire hinge region.

In another embodiment, the Fc domain may contain one or more amino acid insertions, deletions or substitutions that reduce binding to the low affinity inhibitory Fc receptor CD32B (FcγRIIB) and retain wild-type levels of binding to or enhance binding to the low affinity activating Fc receptor CD16A (FcγRIIIA).

Another embodiment includes IgG2-4 hybrids and IgG4 mutants that have reduce binding to FcR which increase their half-life. Representative IG2-4 hybrids and IgG4 mutants are described in Angal, S. et al., Molecular Immunology, 30(1):105-108 (1993); Mueller, J. et al., Molecular Immunology, 34(6): 441-452 (1997); and U.S. Pat. No. 6,982,323 to Wang et al. In some embodiments the IgG1 and/or IgG2 domain is deleted for example, Angal et al. describe IgG1 and IgG2 having serine 241 replaced with a proline.

In some embodiments, the Fc domain contains amino acid insertions, deletions or substitutions that enhance binding to CD16A. A large number of substitutions in the Fc domain of human IgG1 that increase binding to CD16A and reduce binding to CD32B are known in the art and are described in Stavenhagen, et al., Cancer Res, 57(18):8882-90 (2007). Exemplary variants of human IgG1 Fc domains with reduced binding to CD32B and/or increased binding to CD16A contain F243L, R929P, Y300L, V305I or P296L substitutions. These amino acid substitutions may be present in a human IgG1 Fc domain in any combination. In one embodiment, the human IgG1 Fc domain variant contains a F243L, R929P and Y300L substitution. In another embodiment, the human IgG1 Fc domain variant contains a F243L, R929P, Y300L, V305I and P296L substitution. In another embodiment, the human IgG1 Fc domain variant contains an N297Q substitution, as this mutation abolishes FcR binding.

The disclosed fusion proteins optionally contain a peptide or polypeptide linker domain that separates the KLRG1 polypeptide from the second polypeptide. In some embodiments, the linker domain contains the hinge region of an immunoglobulin. In a preferred embodiment, the hinge region is derived from a human immunoglobulin. Suitable human immunoglobulins that the hinge can be derived from include IgG, IgD and IgA. In a preferred embodiment, the hinge region is derived from human IgG. Amino acid sequences of immunoglobulin hinge regions and other domains are well known in the art.

In some embodiments, the leader sequence, the linker (e.g., the hinge region), the second fusion partner (e.g., IgG1 Fc domain), or a combination thereof are substitute for another sequence(s) (e.g., an alternative leader sequence, hinge, Fc domain, etc.). Suitable substitutes are well known in the art. See, for example, U.S. Pat. No. 9,005,616, which is specifically incorporated by reference in its entirety.

3. KLRG1 Nucleic Acids and Cells

Vectors encoding KLRG1 polypeptides, fragments and fusions thereof are also provided. Nucleic acids, such as Accession: NM_001329099.1 or GI: 1041817915, can be inserted into vectors for expression in cells. Thus cells containing and expressing KLRG1 polypeptides, fragments and fusions thereof are also provided. As used herein, a “vector” is a replicon, such as a plasmid, phage, virus or cosmid, into which another DNA segment may be inserted so as to bring about the replication of the inserted segment. Vectors can be expression vectors. An “expression vector” is a vector that includes one or more expression control sequences, and an “expression control sequence” is a DNA sequence that controls and regulates the transcription and/or translation of another DNA sequence.

Nucleic acids in vectors can be operably linked to one or more expression control sequences. As used herein, “operably linked” means incorporated into a genetic construct so that expression control sequences effectively control expression of a coding sequence of interest. Examples of expression control sequences include promoters, enhancers, and transcription terminating regions. A promoter is an expression control sequence composed of a region of a DNA molecule, typically within 100 nucleotides upstream of the point at which transcription starts (generally near the initiation site for RNA polymerase II). To bring a coding sequence under the control of a promoter, it is necessary to position the translation initiation site of the translational reading frame of the polypeptide between one and about fifty nucleotides downstream of the promoter. Enhancers provide expression specificity in terms of time, location, and level. Unlike promoters, enhancers can function when located at various distances from the transcription site. An enhancer also can be located downstream from the transcription initiation site. A coding sequence is “operably linked” and “under the control” of expression control sequences in a cell when RNA polymerase is able to transcribe the coding sequence into mRNA, which then can be translated into the protein encoded by the coding sequence.

Suitable expression vectors include, without limitation, plasmids and viral vectors derived from, for example, bacteriophage, baculoviruses, tobacco mosaic virus, herpes viruses, cytomegalo virus, retroviruses, vaccinia viruses, adenoviruses, and adeno-associated viruses. Numerous vectors and expression systems are commercially available from such corporations as Novagen (Madison, Wis.), Clontech (Palo Alto, Calif.), Stratagene (La Jolla, Calif.), and Invitrogen Life Technologies (Carlsbad, Calif.).

An expression vector can include a tag sequence. Tag sequences, are typically expressed as a fusion with the encoded polypeptide. Such tags can be inserted anywhere within the polypeptide including at either the carboxyl or amino terminus. Examples of useful tags include, but are not limited to, green fluorescent protein (GFP), glutathione S-transferase (GST), polyhistidine, c-myc, hemagglutinin, Flag™ tag (Kodak, New Haven, Conn.), maltose E binding protein and protein A. In some embodiments, a nucleic acid molecule encoding a KLRG1 fusion polypeptide is present in a vector containing nucleic acids that encode one or more domains of an Ig heavy chain constant region, for example, an amino acid sequence corresponding to the hinge, C_{H}2 and C_{H}3 regions of a human immunoglobulin Cγ1 chain.

Vectors containing nucleic acids to be expressed can be transferred into host cells. The term “host cell” is intended to include prokaryotic and eukaryotic cells into which a recombinant expression vector can be introduced. As used herein, “transformed” and “transfected” encompass the introduction of a nucleic acid molecule (e.g., a vector) into a cell by one of a number of techniques. Although not limited to a particular technique, a number of these techniques are well established within the art. Prokaryotic cells can be transformed with nucleic acids by, for example, electroporation or calcium chloride mediated transformation. Nucleic acids can be transfected into mammalian cells by techniques including, for example, calcium phosphate co-precipitation, DEAE-dextran-mediated transfection, lipofection, electroporation, or microinjection. Host cells (e.g., a prokaryotic cell or a eukaryotic cell such as a CHO cell) can be used to, for example, produce the KLRG1 fusion polypeptides described herein.

The vectors described can be used to express KLRG1 in cells. An exemplary vector includes, but is not limited to, an adenoviral vector. One approach includes nucleic acid transfer into primary cells in culture followed by autologous transplantation of the ex vivo transformed cells into the host, either systemically or into a particular organ or tissue. Ex vivo methods can include, for example, the steps of harvesting cells from a subject, culturing the cells, transducing them with an expression vector, and maintaining the cells under conditions suitable for expression of the encoded polypeptides. These methods are known in the art of molecular biology. The transduction step can be accomplished by any standard means used for ex vivo gene therapy, including, for example, calcium phosphate, lipofection, electroporation, viral infection, and biolistic gene transfer. Alternatively, liposomes or polymeric microparticles can be used. Cells that have been successfully transduced then can be selected, for example, for expression of the coding sequence or of a drug resistance gene. The cells then can be lethally irradiated (if desired) and injected or implanted into the subject. In one embodiment, expression vectors containing nucleic acids encoding fusion proteins are transfected into cells that are administered to a subject in need thereof.

In vivo nucleic acid therapy can be accomplished by direct transfer of a functionally active DNA into mammalian somatic tissue or organ in vivo. For example, nucleic acids encoding polypeptides disclosed herein can be administered directly to lymphoid tissues or tumors. Alternatively, lymphoid tissue specific targeting can be achieved using lymphoid tissue-specific transcriptional regulatory elements (TREs) such as a B lymphocyte-, T lymphocyte-, or dendritic cell-specific TRE. Lymphoid tissue specific TREs are known in the art.

Nucleic acids may also be administered in vivo by viral means. Nucleic acid molecules encoding fusion proteins may be packaged into retrovirus vectors using packaging cell lines that produce replication-defective retroviruses, as is well-known in the art. Other virus vectors may also be used, including recombinant adenoviruses and vaccinia virus, which can be rendered non-replicating. In addition to naked DNA or RNA, or viral vectors, engineered bacteria may be used as vectors.

Nucleic acids may also be delivered by other carriers, including liposomes, polymeric micro- and nanoparticles and polycations such as asialoglycoprotein/polylysine.

In addition to virus- and carrier-mediated gene transfer in vivo, physical means well-known in the art can be used for direct transfer of DNA, including administration of plasmid DNA and particle-bombardment mediated gene transfer.

C. Pharmaceutical Compositions

Pharmaceutical compositions including the disclosed KLRG1-binding molecules are provided. Pharmaceutical compositions containing a KLRG1-binding molecule can be for administration by parenteral (intramuscular, intraperitoneal, intravenous (IV) or subcutaneous injection), transdermal (either passively or using iontophoresis or electroporation), or transmucosal (nasal, vaginal, rectal, or sublingual) routes of administration or using bioerodible inserts and can be formulated in dosage forms appropriate for each route of administration.

In some in vivo approaches, the compositions disclosed herein are administered to a subject in a therapeutically effective amount. As used herein the term “effective amount” or “therapeutically effective amount” means a dosage sufficient to treat, inhibit, or alleviate one or more symptoms of the disorder being treated or to otherwise provide a desired pharmacologic and/or physiologic effect. The precise dosage will vary according to a variety of factors such as subject-dependent variables (e.g., age, immune system health, etc.), the disease, and the treatment being effected.

For the disclosed KLRG1-binding molecules, as further studies are conducted, information will emerge regarding appropriate dosage levels for treatment of various conditions in various patients, and the ordinary skilled worker, considering the therapeutic context, age, and general health of the recipient, will be able to ascertain proper dosing. The selected dosage depends upon the desired therapeutic effect, on the route of administration, and on the duration of the treatment desired. Generally, for intravenous injection or infusion, dosage may be lower.

The dosage administered to a patient is typically 0.01 mg/kg to 100 mg/kg of the patient's body weight. The dosage administered to a patient can be, for example, between 0.01 mg/kg and 20 mg/kg, 0.01 mg/kg and 10 mg/kg, 0.01 mg/kg and 5 mg/kg, 0.01 and 2 mg/kg, 0.01 and 1 mg/kg, 0.01 mg/kg and 0.75 mg/kg, 0.01 mg/kg and 0.5 mg/kg, 0.01 mg/kg to 0.25 mg/kg, 0.01 to 0.15 mg/kg, 0.01 to 0.10 mg/kg, 0.01 to 0.05 mg/kg, or 0.01 to 0.025 mg/kg of the patient's body weight. Exemplary specific dosages include, but are not limited to 0.2 mg/kg, 0.3 mg/kg, 1 mg/kg, 3 mg/kg, 6 mg/kg or 10 mg/kg. A dose as low as 0.01 mg/kg is believed to be suitable to have appreciable pharmacodynamic effects. Dose levels of 0.10-1 mg/kg are predicted to be most appropriate. Higher doses (e.g., 1-30 mg/kg) would also be expected to be active.

Generally, human antibodies have a longer half-life within the human body than antibodies from other species due to the immune response to the foreign polypeptides. Thus, lower dosages of human antibodies and less frequent administration is often possible. Further, the dosage and frequency of administration of antibodies of the invention or fragments thereof may be reduced by enhancing uptake and tissue penetration of the antibodies by modifications such as, for example, lipidation.

In certain embodiments, the KLRG1-binding molecule is administered locally, for example by injection directly into a site to be treated. Typically, the injection causes an increased localized concentration of the KLRG1-binding molecule composition which is greater than that which can be achieved by systemic administration. The KLRG1-binding molecule compositions can be combined with a matrix as described below to assist in creating an increased localized concentration of the polypeptide compositions by reducing the passive diffusion of the polypeptides out of the site to be treated.

1. Formulations for Parenteral Administration

In some embodiments, compositions disclosed herein, are administered in an aqueous solution, by parenteral injection or infusion. The formulation may also be in the form of a suspension or emulsion. In general, pharmaceutical compositions are provided including effective amounts of a KLRG1-binding molecule, and optionally include pharmaceutically acceptable diluents, preservatives, solubilizers, emulsifiers, adjuvants and/or carriers. Such compositions optionally include one or more for the following: diluents, sterile water, buffered saline of various buffer content (e.g., Tris-HCl, acetate, phosphate), pH and ionic strength; and additives such as detergents and solubilizing agents (e.g., TWEEN® 20 (polysorbate-20), TWEEN® 80 (polysorbate-80)), anti-oxidants (e.g., ascorbic acid, sodium metabisulfite), and preservatives (e.g., Thimersol, benzyl alcohol) and bulking substances (e.g., lactose, mannitol). Examples of non-aqueous solvents or vehicles are propylene glycol, polyethylene glycol, vegetable oils, such as olive oil and corn oil, gelatin, and injectable organic esters such as ethyl oleate. The formulations may be lyophilized and redissolved/resuspended immediately before use. The formulation may be sterilized by, for example, filtration through a bacteria retaining filter, by incorporating sterilizing agents into the compositions, by irradiating the compositions, or by heating the compositions.

2. Controlled Delivery Polymeric Matrices

The KLRG1-binding molecules disclosed herein can also be administered in controlled release formulations. Controlled release polymeric devices can be made for long term release systemically following implantation of a polymeric device (rod, cylinder, film, disk) or injection (microparticles). The matrix can be in the form of microparticles such as microspheres, where the agent is dispersed within a solid polymeric matrix or microcapsules, where the core is of a different material than the polymeric shell, and the peptide is dispersed or suspended in the core, which may be liquid or solid in nature. Unless specifically defined herein, microparticles, microspheres, and microcapsules are used interchangeably. Alternatively, the polymer may be cast as a thin slab or film, ranging from nanometers to four centimeters, a powder produced by grinding or other standard techniques, or even a gel such as a hydrogel.

Either non-biodegradable or biodegradable matrices can be used for delivery of fusion polypeptides or nucleic acids encoding the fusion polypeptides. These may be natural or synthetic polymers. Synthetic polymers typically have a better characterization of degradation and release profiles. The polymer is selected based on the period over which release is desired. In some cases linear release may be most useful, although in others a pulse release or “bulk release” may provide more effective results. The polymer may be in the form of a hydrogel (typically in absorbing up to about 90% by weight of water), and can optionally be crosslinked with multivalent ions or polymers.

The matrices can be formed by solvent evaporation, spray drying, solvent extraction and other methods known to those skilled in the art. Bioerodible microspheres can be prepared using any of the methods developed for making microspheres for drug delivery, for example, as described by Mathiowitz et al., J Controlled Release, 5:13-22 (1987); Mathiowitz, et al., Reactive Polymers, 6:275-283 (1987); and Mathiowitz, et al., J. appl Polymer Sci, 35:755-774 (1988).

The devices can be formulated for local release to treat the area of implantation or injection—which will typically deliver a dosage that is much less than the dosage for treatment of an entire body—or systemic delivery. These can be implanted or injected subcutaneously, into the muscle, fat, or swallowed.

### III. Method of Use

Methods of using the disclosed KLRG1-binding and KLRG1 ligand-binding molecules are also provided. Uses of such molecules to increase immune responses, retard or prevent tumor growth, inhibit tumor-mediated immune suppression, eliminate tumors, and/or reduce or reverse T cell suppression are disclosed. Also provided are uses of such molecules in the diagnosis and the treatment of cancer and other diseases.

In one embodiment, antibodies produced by one of the group of hybridomas K1A6, K1B9, K1F7, K2H11, K3G4, K3H11, K4C10, K4F4, K4F5, K4F5A, K7C12, K8F2, K9H1, or K10D5, or variants thereof can be used to increase immune responses, retard or prevent tumor growth, inhibit tumor-mediated immune suppression, eliminate tumors, and/or reduce or reverse T cell suppression.

Binding of function blocking or reducing anti-KLRG1 antibodies to KLRG1 can result in reducing, blocking, antagonizing, attenuating, or in completely abolishing the ability of KLRG1 to bind to one or more it's ligands and therefore decrease or prevent inhibitory immune signaling

Methods of reducing immune suppression and/or increasing an immune response, most typically by administering to a subject in need thereof an effective amount of anti-KLRG1 function blocking antibody, are provided.

Suitable antibodies, polypeptides, and fusion proteins are disclosed herein and can be further selected by in vitro assays including but not limited to: proliferation, cytotoxicity, migration, adhesion, angiogenesis, cell-cell communication, apoptosis, transport, signal transduction, and in vivo assays such as the inhibition of tumor growth. One embodiment provides antibodies produced by one of the group of hybridomas K1A6, K1B9, K1F7, K2H11, K3G4, K3H11, K4C10, K4F4, K4F5, K4F5A, K7C12, K8F2, K9H1, or K10D5. In one embodiment, the antibody includes, but is not limited to an antibody or an antigen binding fragment thereof having a light chain with at least 50%, 60%, 70%, 80%, 85%, 90%, 95%, 99%, or 100% sequence identity to SEQ ID NO:4, 14, 24, 34, 44, 57, 67, 77, 87, 95, 105, or 112 and a heavy chain having at least 50%, 60%, 70%, 80%, 85%, 90%, 95%, 99%, or 100% sequence identity to SEQ ID NO:9, 19, 29, 49, 55, 62, 72, 82, 90, 100, 108, or 116. In another embodiment, the antibody includes but is not limited to a humanized antibody or antigen-binding fragment having a light chain with at least 50%, 60%, 70%, 80%, 85%, 90%, 95%, 99%, or 100% sequence identity to SEQ ID NO:126, 127, 128, or 129, and a heavy chain having at least 50%, 60%, 70%, 80%, 85%, 90%, 95%, 99%, or 100% sequence identity to SEQ ID NO:137, 139, 141, 143, or 145. In yet another embodiment, the antibody includes but is not limited to a humanized antibody or antigen-binding fragment having a light chain with at least 50%, 60%, 70%, 80%, 85%, 90%, 95%, 99%, or 100% sequence identity to SEQ ID NO:126, 127, 128, or 129, and a heavy chain having at least 50%, 60%, 70%, 80%, 85%, 90%, 95%, 99%, or 100% sequence identity to SEQ ID NO:138, 140, 142, 144, or 146.

The antibodies provided herein can also be useful in diagnostic and research applications. For example, non-neutralizing antibodies can bind to the specific antigen without inhibiting the receptor-binding or biological activity of the antigen, and can be used in capture assays and other pull-downs (e.g., ELISA). Neutralizing (e.g., function blocking, antagonist) antibodies can be useful in competitive binding assays.

Antibodies can also be used to quantify the KLRG1 polypeptides or its ligand.

A. Immune Response Increasing Molecules

1. Therapeutic and Prophylactic Uses

Therapeutic and/or prophylactic use of molecules (especially antibodies or their antigen-binding fragments) that immunospecifically bind human KLRG1 and/or KLRG1 ligand-binding molecules and that are capable of reducing the binding between KLRG1 and one or more of its ligands and/or counter-receptors are provided (e.g., functional blocking molecules, antagonist molecules).

In some embodiments, the molecules reduce or prevent binding between KLRG1 and a cadherins endogenously expressed by the cells of a subject or present in soluble form, and reduce or prevent KLRG1 mediated signal transduction. Additionally or alternatively, the molecules can reduce or prevent binding between KLRG1 and a counter-receptor thereof, and reduce or prevent KLRG1-mediated signal transduction and/or signal transduction through the counter-receptor. For example, the disclosed molecules can bind to antigens at one or more sites on KLRG1 and/or binding site important for binding to a KLRG1 ligand.

Additionally, the disclosed antagonist molecules can be used to induce, increase, or enhance T cell proliferation, cytotoxic killing activity (i.e. perforins, granzymes), FasL-Fas mediated killing, and/or cytokine mediated effector activity (i.e. cytokines such as interferon-gamma). In some embodiments, the T cell response is induced by reducing or preventing KLRG1 from binding to a KLRG1 ligand. Up-modulation of the immune system is particularly desirable in the treatment of cancers and chronic infections, and thus the disclosed compositions can be used in the treatment of such disorders.

2. Subjects to be Treated

a. Treatment of Cancer

The disclosed function reducing compositions and methods can be used to treat cancer. In one embodiment, antibodies produced by one of the group of hybridomas K1A6, K1B9, K1F7, K2H11, K3G4, K3H11, K4C10, K4F4, K4F5, K7C12, K8F2, K9H1, or K10D5, can be used to treat cancer. Generally, the methods include stimulating or enhancing an immune response to cancer, reducing or preventing tumor growth or progression, or a combination thereof in the subject by administering to the subject an amount of a KLRG1 binding molecule. The method can reduce one or more symptoms of the cancer.

Cancer cells acquire a characteristic set of functional capabilities during their development, albeit through various mechanisms. Such capabilities include evading apoptosis, self-sufficiency in growth signals, insensitivity to anti-growth signals, tissue invasion/metastasis, limitless replicative potential, and sustained angiogenesis. The term “cancer cell” is meant to encompass both pre-malignant and malignant cancer cells. In some embodiments, cancer refers to a benign tumor, which has remained localized. In other embodiments, cancer refers to a malignant tumor, which has invaded and destroyed neighboring body structures and spread to distant sites. In yet other embodiments, the cancer is associated with a specific cancer antigen (e.g., pan-carcinoma antigen (KS 1/4), ovarian carcinoma antigen (CA125), prostate specific antigen (PSA), carcinoembryonic antigen (CEA), CD19, CD20, HER2/neu, etc.).

The methods and compositions disclosed herein are useful in the treatment or prevention of a variety of cancers or other abnormal proliferative diseases, including (but not limited to) the following: carcinoma, including that of the bladder, breast, colon, kidney, liver, lung, ovary, pancreas, stomach, cervix, thyroid and skin; including squamous cell carcinoma; hematopoietic tumors of lymphoid lineage, including leukemia, acute lymphocytic leukemia, acute lymphoblastic leukemia, B-cell lymphoma, T-cell lymphoma, Berketts lymphoma; hematopoietic tumors of myeloid lineage, including acute and chronic myelogenous leukemias and promyelocytic leukemia; tumors of mesenchymal origin, including fibrosarcoma and rhabdomyoscarcoma; other tumors, including melanoma, seminoma, tetratocarcinoma, neuroblastoma and glioma; tumors of the central and peripheral nervous system, including astrocytoma, neuroblastoma, glioma, and schwannomas; tumors of mesenchymal origin, including fibrosarcoma, rhabdomyoscarama, and osteosarcoma; and other tumors, including melanoma, xenoderma pegmentosum, keratoactanthoma, seminoma, thyroid follicular cancer and teratocarcinoma.

Cancers caused by aberrations in apoptosis can also be treated by the disclosed methods and compositions. Such cancers may include, but are not be limited to, follicular lymphomas, carcinomas with p53 mutations, hormone dependent tumors of the breast, prostate and ovary, and precancerous lesions such as familial adenomatous polyposis, and myelodysplastic syndromes. In specific embodiments, malignancy or dysproliferative changes (such as metaplasias and dysplasias), or hyperproliferative disorders, are treated or prevented by the methods and compositions in the ovary, bladder, breast, colon, lung, skin, pancreas, or uterus. In other specific embodiments, sarcoma, melanoma, or leukemia is treated or prevented by the methods and compositions.

Specific cancers and related disorders that can be treated or prevented by methods and compositions disclosed herein include, but are not limited to, leukemias including, but not limited to, acute leukemia, acute lymphocytic leukemia, acute myelocytic leukemias such as myeloblastic, promyelocytic, myelomonocytic, monocytic, erythroleukemia leukemias and myelodysplastic syndrome, chronic leukemias such as but not limited to, chronic myelocytic (granulocytic) leukemia, chronic lymphocytic leukemia, hairy cell leukemia; polycythemia vera; lymphomas such as, but not limited to, Hodgkin's disease or non-Hodgkin's disease lymphomas (e.g., diffuse anaplastic lymphoma kinase (ALK) negative, large B-cell lymphoma (DLBCL); diffuse anaplastic lymphoma kinase (ALK) positive, large B-cell lymphoma (DLBCL); anaplastic lymphoma kinase (ALK) positive, ALK+ anaplastic large-cell lymphoma (ALCL), acute myeloid lymphoma (AML)); multiple myelomas such as, but not limited to, smoldering multiple myeloma, nonsecretory myeloma, osteosclerotic myeloma, plasma cell leukemia, solitary plasmacytoma and extramedullary plasmacytoma; Waldenstrom's macroglobulinemia; monoclonal gammopathy of undetermined significance; benign monoclonal gammopathy; heavy chain disease; bone and connective tissue sarcomas such as, but not limited to, bone sarcoma, osteosarcoma, chondrosarcoma, Ewing's sarcoma, malignant giant cell tumor, fibrosarcoma of bone, chordoma, periosteal sarcoma, soft-tissue sarcomas, angiosarcoma (hemangiosarcoma), fibrosarcoma, Kaposi's sarcoma, leiomyosarcoma, liposarcoma, lymphangiosarcoma, neurilemmoma, rhabdomyosarcoma, synovial sarcoma; brain tumors including but not limited to, glioma, astrocytoma, brain stem glioma, ependymoma, oligodendroglioma, nonglial tumor, acoustic neurinoma, craniopharyngioma, medulloblastoma, meningioma, pineocytoma, pineoblastoma, primary brain lymphoma; breast cancer including, but not limited to, adenocarcinoma, lobular (small cell) carcinoma, intraductal carcinoma, medullary breast cancer, mucinous breast cancer, tubular breast cancer, papillary breast cancer, Paget's disease, and inflammatory breast cancer; adrenal cancer, including but not limited to, pheochromocytom and adrenocortical carcinoma; thyroid cancer such as but not limited to papillary or follicular thyroid cancer, medullary thyroid cancer and anaplastic thyroid cancer; pancreatic cancer, including but not limited to, insulinoma, gastrinoma, glucagonoma, vipoma, somatostatin-secreting tumor, and carcinoid or islet cell tumor; pituitary cancers including but not limited to, Cushing's disease, prolactin-secreting tumor, acromegaly, and diabetes insipius; eye cancers including, but not limited to, ocular melanoma such as iris melanoma, choroidal melanoma, and cilliary body melanoma, and retinoblastoma; vaginal cancers, including, but not limited to, squamous cell carcinoma, adenocarcinoma, and melanoma; vulvar cancer, including but not limited to, squamous cell carcinoma, melanoma, adenocarcinoma, basal cell carcinoma, sarcoma, and Paget's disease; cervical cancers including, but not limited to, squamous cell carcinoma, and adenocarcinoma; uterine cancers including, but not limited to, endometrial carcinoma and uterine sarcoma; ovarian cancers including, but not limited to, ovarian epithelial carcinoma, borderline tumor, germ cell tumor, and stromal tumor; esophageal cancers including, but not limited to, squamous cancer, adenocarcinoma, adenoid cyctic carcinoma, mucoepidermoid carcinoma, adenosquamous carcinoma, sarcoma, melanoma, plasmacytoma, verrucous carcinoma, and oat cell (small cell) carcinoma; stomach cancers including, but not limited to, adenocarcinoma, fungating (polypoid), ulcerating, superficial spreading, diffusely spreading, malignant lymphoma, liposarcoma, fibrosarcoma, and carcinosarcoma; colon cancers; rectal cancers; liver cancers including, but not limited to, hepatocellular carcinoma and hepatoblastoma, gallbladder cancers including, but not limited to, adenocarcinoma; cholangiocarcinomas including, but not limited to, papillary, nodular, and diffuse; lung cancers including but not limited to, non-small cell lung cancer, squamous cell carcinoma (epidermoid carcinoma), adenocarcinoma, large-cell carcinoma and small-cell lung cancer; testicular cancers including, but not limited to, germinal tumor, seminoma, anaplastic, classic (typical), spermatocytic, nonseminoma, embryonal carcinoma, teratoma carcinoma, choriocarcinoma (yolk-sac tumor), prostate cancers including, but not limited to, adenocarcinoma, leiomyosarcoma, and rhabdomyosarcoma; penal cancers; oral cancers including, but not limited to, squamous cell carcinoma; basal cancers; salivary gland cancers including, but not limited to, adenocarcinoma, mucoepidermoid carcinoma, and adenoidcystic carcinoma; pharynx cancers including, but not limited to, squamous cell cancer, and verrucous; skin cancers including, but not limited to, basal cell carcinoma, squamous cell carcinoma and melanoma, superficial spreading melanoma, nodular melanoma, lentigo malignant melanoma, acral lentiginous melanoma; kidney cancers including, but not limited to, renal cell cancer, adenocarcinoma, hypernephroma, fibrosarcoma, transitional cell cancer (renal pelvis and/or uterer); Wilms' tumor; bladder cancers including, but not limited to, transitional cell carcinoma, squamous cell cancer, adenocarcinoma, carcinosarcoma. In addition, cancers include myxosarcoma, osteogenic sarcoma, endotheliosarcoma, lymphangioendotheliosarcoma, mesothelioma, synovioma, hemangioblastoma, epithelial carcinoma, cystadenocarcinoma, bronchogenic carcinoma, sweat gland carcinoma, sebaceous gland carcinoma, papillary carcinoma and papillary adenocarcinomas (for a review of such disorders, see Fishman et al., Medicine, 2d Ed., J.B. Lippincott Co., Philadelphia (1985), and Murphy et al., “Informed Decisions: The Complete Book of Cancer Diagnosis, Treatment, and Recovery”, Viking Penguin, Penguin Books U.S.A., Inc., United States of America (1997)).

b. mAb Mediated Depletion of KLRG1+ Cells

Some studies have indicated that the KLRG1 is primarily expressed on terminally-differentiated T cells and regulatory T cells. One embodiment provides a method in which the anti-KLRG1 antibodies are used to deplete KLRG1+ cells and thereby improve anti-tumor immunity. The method includes administering to a patient in need thereof, an effective amount of an anti-KLRG1 antibody or fragment thereof to deplete KLRG1+ cells in the subject.

c. Treatment of Infections

The disclosed function reducing compositions and methods can be used to treat infections and infectious diseases. In one embodiment, antibodies produced by one of the group of hybridomas K1A6, K1B9, K1F7, K2H11, K3G4, K3H11, K4C10, K4F4, K4F5, K7C12, K8F2, K9H1, or K10D5, can be used to treat infections. Generally, the methods include stimulating or enhancing an immune response to an infection causing agent, reducing or preventing infectious disease progression, or a combination thereof in the subject by administering to the subject an amount of a KLRG1 binding molecule. The method can reduce one or more symptoms of the infection.

The infection or disease can be caused by a bacterium, virus, protozoan, helminth, or other microbial pathogen that enters intracellularly and is attacked, i.e., by cytotoxic T lymphocytes.

The infection or disease can be acute or chronic. An acute infection is typically an infection of short duration. During an acute microbial infection, immune cells begin expressing immunomodulatory receptors. Accordingly, in some embodiments, the method includes increasing an immune stimulatory response against an acute infection.

The infection can be caused by, for example, but not limited to Candida albicans, Listeria monocytogenes, Streptococcus pyogenes, Streptococcus pneumoniae, Neisseria meningitidis, Staphylococcus aureus, Escherichia coli, Acinetobacter baumannii, Pseudomonas aeruginosa or Mycobacterium.

In some embodiments, the disclosed compositions are used to treat chronic infections, for example infections in which T cell exhaustion or T cell anergy has occurred causing the infection to remain with the host over a prolonged period of time.

Exemplary infections to be treated are chronic infections cause by a hepatitis virus, a human immunodeficiency virus (HIV), a human T-lymphotrophic virus (HTLV), a herpes virus, an Epstein-Barr virus, or a human papilloma virus.

Because viral infections are cleared primarily by T cells, an increase in T-cell activity would be therapeutically useful in situations where more rapid or thorough clearance of an infective viral agent would be beneficial to an animal or human subject. Thus, the disclosed compositions can be administered for the treatment of local or systemic viral infections, including, but not limited to, immunodeficiency (e.g., HIV), papilloma (e.g., HPV), herpes (e.g., HSV), encephalitis, influenza (e.g., human influenza virus A), and common cold (e.g., human rhinovirus) and other viral infections, caused by, for example, HTLV, hepatitis virus, respiratory syncytial virus, vaccinia virus, and rabies virus. The molecules can be administered topically to treat viral skin diseases such as herpes lesions or shingles, or genital warts. The molecules can also be administered systemically to treat systemic viral diseases, including, but not limited to, AIDS, influenza, the common cold, or encephalitis.

Representative infections that can be treated, include but are not limited to infections cause by microorganisms including, but not limited to, Actinomyces, Anabaena, Bacillus, Bacteroides, Bdellovibrio, Bordetella, Borrelia, Campylobacter, Caulobacter, Chlamydia, Chlorobium, Chromatium, Clostridium, Corynebacterium, Cytophaga, Deinococcus, Escherichia, Francisella, Halobacterium, Heliobacter, Haemophilus, Hemophilus influenza type B (HIB), Hyphomicrobium, Legionella, Leptspirosis, Listeria, Meningococcus A, B and C, Methanobacterium, Micrococcus, Myobacterium, Mycoplasma, Myxococcus, Neisseria, Nitrobacter, Oscillatoria, Prochloron, Proteus, Pseudomonas, Phodospirillum, Rickettsia, Salmonella, Shigella, Spirillum, Spirochaeta, Staphylococcus, Streptococcus, Streptomyces, Sulfolobus, Thermoplasma, Thiobacillus, and Treponema, Vibrio, Yersinia, Cryptococcus neoformans, Histoplasma capsulatum, Candida albicans, Candida tropicalis, Nocardia asteroides, Rickettsia ricketsii, Rickettsia typhi, Mycoplasma pneumoniae, Chlamydial psittaci, Chlamydial trachomatis, Plasmodium falciparum, Trypanosoma brucei, Entamoeba histolytica, Toxoplasma gondii, Trichomonas vaginalis and Schistosoma mansoni.

Other microorganisms that can be treated using the disclosed compositions and methods include, bacteria, such as those of Klebsiella, Serratia, Pasteurella; pathogens associated with cholera, tetanus, botulism, anthrax, plague, and Lyme disease; or fungal or parasitic pathogens, such as Candida (albicans, krusei, glabrata, tropicalis, etc.), Cryptococcus, Aspergillus (fumigatus, niger, etc.), Genus Mucorales (mucor, absidia, rhizophus), Sporothrix (schenkii), Blastomyces (dermatitides), Paracoccidioides (brasiliensis), Coccidioides (immitis) and Histoplasma (capsulatuma), Entamoeba, histolytica, Balantidium coli, Naegleria fowleri, Acanthamoeba sp., Giardia lambia, Cryptosporidium sp., Pneumocystis carinii, Plasmodium vivax, Babesia micron, Trypanosoma brucei, Trypanosoma cruzi, Toxoplasma gondi, etc.), Sporothrix, Blastomyces, Paracoccidioides, Coccidioides, Histoplasma, Entamoeba, Histolytica, Balantidium, Naegleria, Acanthamoeba, Giardia, Cryptosporidium, Pneumocystis, Plasmodium, Babesia, or Trypanosoma, etc.

B. Immune Response Reducing Molecules

1. Therapeutic and Prophylactic Uses

Therapeutic and/or prophylactic use of molecules (especially antibodies or their antigen-binding fragments) that bind human KLRG1 or to a ligand thereof, and that are capable of increasing or enhancing the binding between KLRG1 and one or more of its ligands and/or counter-receptors are provided (e.g., agonist molecules) or directly increasing or enhancing KLRG1 or KLRG1 counter-receptor mediated signal transduction are also provided.

As discussed above, interactions between KLRG1 and its ligands can inhibit the proliferation of immune cells such as T cells and NK cells. Thus, in some embodiments, the administration of function activating KLRG1-binding molecules or function activating KLRG1-ligand-binding molecules to a subject down-modulates the immune system of the subject by inducing or otherwise agonizing KLRG1-ligand and/or counter-receptor binding/interaction or directly simulating KLRG1 or KLRG1 counter-receptor signal transduction. Activating signal transduction through KLRG1 inhibits both cytokine production and NK cell-mediated cytotoxicity.

Additionally, the disclosed agonist KLRG1 binding and KLRG1 ligand-binding molecules can be used to reduce or decrease T cell and/or NK cell proliferation. In some embodiments, the immune cell response is induced by increasing or enhancing KLRG1 binding to a counter-receptor on the immune cell. Down-modulation of the immune system is particularly desirable in the treatment of inflammatory and autoimmune diseases and disorder and to treat or prevent graft rejection and/or graft verse host disease, and thus the disclosed compositions can be used in the treatment of such disorders.

a. Inflammatory Responses

In some embodiments, KLRG1 agonist molecules that increase or induce KLRG1 signal transduction are used for treating or alleviating one or more symptoms of inflammation, for example acute, chronic, or persistent inflammation.

An immune response including inflammation can be inhibited or reduced in a subject, preferably a human, by administering an effective amount the KLRG1-binding molecule to inhibit or reduce the biological activity of an immune cell (e.g., T cells or B cells) or to reduce the amounts of proinflammatory molecules at a site of inflammation. Exemplary proinflammatory molecules include, but are not limited to, IL-1β, TNF-α, IFN-γ, IL-18, IL-17, IL-6, IL-23, IL-22, IL-21, and MMPs.

b. Hyper-Inflammatory Response

In some embodiments, the KLRG1-binding molecules slow down the immune system. For example, a KLRG1-binding molecule can be used to control an immune stimulatory response to an infection that is causing damage healthy tissues through a hyper-inflammatory response. Accordingly, in some embodiments, the agents are administered to a subject with an infection that is also undergoing a hyper-inflammatory response. In such cases, controlling excessive immune responses can be beneficial to the subject.

c. Inflammatory and Autoimmune Diseases/Disorders

The disclosed agonist molecules can also be used to treat inflammatory or autoimmune diseases and disorders. Representative inflammatory or autoimmune diseases/disorders that can be treated include, but are not limited to, rheumatoid arthritis, systemic lupus erythematosus, alopecia areata, anklosing spondylitis, antiphospholipid syndrome, autoimmune Addison's disease, autoimmune hemolytic anemia, autoimmune hepatitis, autoimmune inner ear disease, autoimmune lymphoproliferative syndrome (alps), autoimmune thrombocytopenic purpura (ATP), Behcet's disease, bullous pemphigoid, cardiomyopathy, celiac sprue-dermatitis, chronic fatigue syndrome immune deficiency, syndrome (CFIDS), chronic inflammatory demyelinating polyneuropathy, cicatricial pemphigoid, cold agglutinin disease, Crest syndrome, Crohn's disease, Dego's disease, dermatomyositis, dermatomyositis—juvenile, discoid lupus, essential mixed cryoglobulinemia, fibromyalgia—fibromyositis, grave's disease, guillain-barre, hashimoto's thyroiditis, idiopathic pulmonary fibrosis, idiopathic thrombocytopenia purpura (ITP), Iga nephropathy, insulin dependent diabetes (Type I), juvenile arthritis, Meniere's disease, mixed connective tissue disease, multiple sclerosis, myasthenia gravis, pemphigus vulgaris, pernicious anemia, polyarteritis nodosa, polychondritis, polyglancular syndromes, polymyalgia rheumatica, polymyositis and dermatomyositis, primary agammaglobulinemia, primary biliary cirrhosis, psoriasis, Raynaud's phenomenon, Reiter's syndrome, rheumatic fever, sarcoidosis, scleroderma, Sjogren's syndrome, stiff-man syndrome, Takayasu arteritis, temporal arteritis/giant cell arteritis, ulcerative colitis, uveitis, vasculitis, vitiligo, and Wegener's granulomatosis.

In some embodiments the inflammation or autoimmune disease is caused by a pathogen, or is the result of an infection.

d. Transplants

The agonist KLRG1-binding molecules can be used for reducing or inhibiting transplant rejection in a subject, preferably a human subject. Transplant rejection can be inhibited or reduced in a subject by administering an effective amount of an agonist KLRG1 binding molecule to inhibit or reduce the biological activity of an immune cell or to reduce the amounts of proinflammatory cytokines or other molecules associated with or that promote inflammation at a site of transplant.

The transplanted material can be cells, tissues, organs, limbs, digits or a portion of the body, preferably the human body. The transplants are typically allogenic or xenogenic. An agonist KLRG1 molecule is typically administered to a subject in an effective amount to reduce or inhibit transplant rejection. The molecule can be administered systemically or locally by any acceptable route of administration. In some embodiments, the molecules are administered to a site of transplantation prior to, at the time of, or following transplantation.

The molecules can be administered directly to cells, tissue or organ to be transplanted ex vivo. In one embodiment, the transplant material is contacted with a KLRG1-binding molecule prior to transplantation, after transplantation, or both.

In other embodiments, a KLRG1-binding molecule is administered to immune tissues or organs, such as lymph nodes or the spleen.

i. Cells

Populations of any types of cells can be transplanted into a subject. The cells can be homogenous or heterogeneous. Heterogeneous means the cell population contains more than one type of cell. Exemplary cells include progenitor cells such as stem cells and pluripotent cells which can be harvested from a donor and transplanted into a subject. The cells are optionally treated ex vivo prior to transplantation. The cells can be autologous or heterologous cells.

ii. Tissues

Any tissue can be used as a transplant. Exemplary tissues include skin, adipose tissue, cardiovascular tissue such as veins, arteries, capillaries, valves; neural tissue, bone marrow, pulmonary tissue, ocular tissue such as corneas and lens, cartilage, bone, and mucosal tissue. The tissue can be modified as discussed above.

iii. Organs

Exemplary organs that can be used for transplant include, but are not limited to kidney, liver, heart, spleen, bladder, lung, stomach, eye, tongue, pancreas, intestine, etc. The organ to be transplanted can also be modified prior to transplantation as discussed above.

One embodiment provides a method of inhibiting or reducing chronic transplant rejection in a subject by administering an effective amount of a KLRG1-binding molecule to inhibit or reduce chronic transplant rejection relative to a control.

e. Graft-Versus-Host Disease (GVHD)

The agonist KLRG1 molecules can also be used to treat graft-versus-host disease (GVHD) by administering an effective amount a KLRG1-binding molecule to alleviate one or more symptoms associated with GVHD. GVHD is a major complication associated with allogeneic hematopoietic stem cell transplantation in which functional immune cells in the transplanted marrow recognize the recipient as “foreign” and mount an immunologic attack. It can also take place in a blood transfusion under certain circumstances. Symptoms of GVD include skin rash or change in skin color or texture, diarrhea, nausea, abnormal liver function, yellowing of the skin, increased susceptibility to infection, dry, irritated eyes, and sensitive or dry mouth.

f. Diabetes

The agonist KLRG1-binding molecules can also be used to treat diabetes. The method includes transplanting insulin producing cells in a subject and administering to the subject an effective amount of a molecule to reduce or inhibit transplant rejection. Preferably the insulin producing cells are beta cells or islet cells. In certain embodiments, the insulin producing cells are recombinant cells engineered to produce insulin.

The insulin producing cells can be encapsulated within a matrix, such as a polymeric matrix, using suitable polymers, including, but not limited to alginate, agarose, hyaluronic acid, collagen, synthetic monomers, albumin, fibrinogen, fibronectin, vitronectin, laminin, dextran, dextran sulfate, chondroitin sulfate, dermatan sulfate, keratan sulfate, chitin, chitosan, heparan, heparan sulfate, or a combination thereof.

C. Targeting and Detection

The disclosed KLRG1 binding and KLRG1 ligand-binding molecules, regardless of their effect on KLRG1 function, can be used for delivering therapeutic cargo and/or detecting the presence of KLRG1 or a ligand thereof, respectively, on cells or tissue. For example, the KLRG1 binding and KLRG1 ligand-binding molecules can be conjugated to a biological molecule of interest to form a conjugate. Cargo including pharmacologically active molecules such as inorganic and organic molecules, pharmaceutical agents, drugs, peptides, proteins, genetic material, etc. can be conjugated the KLRG1 binding or KLRG1 ligand-binding molecule, which can then target the cargo to cells or tissue expressing KLRG1 or a ligand thereof, respectively. KLRG1 molecules can be chemically linked to a polypeptide by a peptide bond or by a chemical or peptide linker molecule. Methods for attaching a drug or other small molecule pharmaceutical to an antibody fragment are well known and include bifunctional chemical linkers such as N-succinimidyl (4-iodoacetyl)-aminobenzoate; sulfosuccinimidyl(4-iodoacetyl)-aminobenzoate; 4-succinimidyl-oxycarbonyl-.A-inverted.-(2-pyridyldithio) toluene; sulfosuccinimidyl-6-[.alpha.-methyl-.A-inverted.-(pyridyldithiol)-toluami-do] hexanoate; N-succinimidyl-3-(−2-pyridyldithio)-proprionate; succinimidyl-6-[3 (+2-pyridyldithio)-proprionamido] hexanoate; sulfosuccinimidyl-6-[3 (+2-pyridyldithio)-propionamido] hexanoate; 3-(2-pyridyldithio)-propionyl hydrazide, Ellman's reagent, dichlorotriazinic acid, S-(2-thiopyridyl)-L-cysteine, and the like.

Fusion proteins can be designed to place the protein of interest at the amino or carboxy terminus of either the antibody heavy or light chain, though the entire heavy chain may not be required. Potential configurations include the use of truncated portions of the heavy and light chain with or without spacer sequences as needed to maintain the functional integrity of the attached protein.

Alternatively, a universal carrier system can be devised. For example, various proteins or DNA can be conjugated to a common carrier such as protein A, poly-L-lysine, hex-histidine, and the like. The conjugated carrier will then form a complex with KLRG1-binding or KLRG1 ligand-binding molecules. A small portion of the carrier molecule that is responsible for binding immunoglobulin could be used as the carrier. Other similar configurations include design of carriers that interact with proteins engineered into the antibody heavy or light chain.

In some embodiments, KLRG1 binding or KLRG1 ligand-binding molecules are conjugated or otherwise incorporated into or onto a nanocarrier to target the nanocarrier to the KLRG1 or KLRG1 ligand positive cells. The nanocarrier, for example, micro- or nano-polymeric particles, liposomes, nanotubes, etc., can include an active agent for delivery to the KLRG1 or KLRG1 ligand positive cells or their microenvironment.

Likewise, the KLRG1 binding or KLRG1 ligand-binding molecules can be conjugated with a detectable marker or can be unconjugated and detected with a secondary reagent to detect KLRG1 or KLRG1 ligand expression, respectively, in vitro or in vivo. Thus the molecules can be used for imagine, immunohistochemistry, and other assays.

### IV. Combination Therapies

The disclosed KLRG1-binding and KLRG1 ligand-binding molecules can be administered to a subject in need thereof alone or in combination with one or more additional therapeutic agents. In some embodiments, the KLRG1-binding or KLRG1 ligand-binding molecule and the additional therapeutic agent are administered separately, but simultaneously. The KLRG1-binding or KLRG1 ligand-binding molecule and the additional therapeutic agent can also be administered as part of the same composition. In other embodiments, the KLRG1-binding or KLRG1 ligand-binding molecule and the second therapeutic agent are administered separately and at different times, but as part of the same treatment regime.

The subject can be administered a first therapeutic agent 1, 2, 3, 4, 5, 6, or more hours, or 1, 2, 3, 4, 5, 6, 7, or more days before administration of a second therapeutic agent. In some embodiments, the subject can be administered one or more doses of the first agent every 1, 2, 3, 4, 5, 6 7, 14, 21, 28, 35, or 48 days prior to a first administration of second agent. The KLRG1-binding or KLRG1 ligand-binding molecule can be the first or the second therapeutic agent. In some embodiments, one or more KLRG1 binding molecules and one or more KLRG1 ligand-binding molecules are administered in combination.

The KLRG1-binding and/or KLRG1 ligand-binding molecule and the additional therapeutic agent can be administered as part of a therapeutic regimen. For example, if a first therapeutic agent can be administered to a subject every fourth day, the second therapeutic agent can be administered on the first, second, third, or fourth day, or combinations thereof. The first therapeutic agent or second therapeutic agent may be repeatedly administered throughout the entire treatment regimen.

Exemplary molecules include, but are not limited to, cytokines, chemotherapeutic agents, radionuclides, other immunotherapeutics, enzymes, antibiotics, antivirals (especially protease inhibitors alone or in combination with nucleosides for treatment of HIV or Hepatitis B or C), anti-parasites (helminths, protozoans), growth factors, growth inhibitors, hormones, hormone antagonists, antibodies and bioactive fragments thereof (including humanized, single chain, and chimeric antibodies), antigen and vaccine formulations (including adjuvants), peptide drugs, anti-inflammatories, ligands that bind to Toll-Like Receptors (including but not limited to polyinosinic:polycytidylic acid (polyI:C) and CpG oligonucleotides) to activate the innate immune system, molecules that mobilize and optimize the adaptive immune system, other molecules that activate or up-regulate the action of cytotoxic T lymphocytes, natural killer cells and helper T-cells, and other molecules that deactivate or down-regulate suppressor or regulatory T-cells.

The additional therapeutic agents are selected based on the condition, disorder or disease to be treated. For example, the KLRG1-binding molecule can be co-administered with one or more additional agents that function to enhance or promote an immune response or reduce or inhibit an immune response.

One embodiment provides a method for treating cancer or reducing tumor burden in a subject in need thereof by administering and anti-KLRG1 antibody or fragment thereof in combination or alternation with a 4-1BB monoclonal antibody. Two studies have suggested that 4-1BB mAb mediated T cell activation induces KLRG1+ effector cells that infiltrate tumors (Choi et al., 2007; Curran et al., 2013). It is likely that KLRG1 is upregulated after activation through 4-1BB signaling, and may potentially exert inhibitory functions. With this in mind, combination therapy with 4-1BB and KLRG1 mAb may have synergistic effects.

A. Chemotherapeutic Agents

The KLRG1-binding and KLRG1 ligand-binding molecules can be combined with one or more chemotherapeutic agents and pro-apoptotic agents. Representative chemotherapeutic agents include, but are not limited to amsacrine, bleomycin, busulfan, capecitabine, carboplatin, carmustine, chlorambucil, cisplatin, cladribine, clofarabine, crisantaspase, cyclophosphamide, cytarabine, dacarbazine, dactinomycin, daunorubicin, docetaxel, doxorubicin, epirubicin, etoposide, fludarabine, fluorouracil, gemcitabine, hydroxycarbamide, idarubicin, ifosfamide, irinotecan, leucovorin, liposomal doxorubicin, liposomal daunorubicin, lomustine, melphalan, mercaptopurine, mesna, methotrexate, mitomycin, mitoxantrone, oxaliplatin, paclitaxel, pemetrexed, pentostatin, procarbazine, raltitrexed, satraplatin, streptozocin, tegafur-uracil, temozolomide, teniposide, thiotepa, tioguanine, topotecan, treosulfan, vinblastine, vincristine, vindesine, vinorelbine, or a combination thereof. Representative pro-apoptotic agents include, but are not limited to fludarabinetaurosporine, cycloheximide, actinomycin D, lactosylceramide, 15d-PGJ(2) and combinations thereof.

B. Other Immunomodulators

1. PD-1 Antagonists

In some embodiments, KLRG1-binding or KLRG1 ligand-binding molecules are co-administered with a PD-1 antagonist. Programmed Death-1 (PD-1) is a member of the CD28 family of receptors that delivers a negative immune response when induced on T cells. Contact between PD-1 and one of its ligands (B7-H1 or B7-DC) induces an inhibitory response that decreases T cell multiplication and/or the strength and/or duration of a T cell response. Suitable PD-1 antagonists are described in U.S. Pat. Nos. 8,114,845, 8,609,089, and 8,709,416, which are specifically incorporated by reference herein in their entities, and include compounds or agents that either bind to and block a ligand of PD-1 to interfere with or inhibit the binding of the ligand to the PD-1 receptor, or bind directly to and block the PD-1 receptor without inducing inhibitory signal transduction through the PD-1 receptor.

In some embodiments, the PD-1 receptor antagonist binds directly to the PD-1 receptor without triggering inhibitory signal transduction and also binds to a ligand of the PD-1 receptor to reduce or inhibit the ligand from triggering signal transduction through the PD-1 receptor. By reducing the number and/or amount of ligands that bind to PD-1 receptor and trigger the transduction of an inhibitory signal, fewer cells are attenuated by the negative signal delivered by PD-1 signal transduction and a more robust immune response can be achieved.

It is believed that PD-1 signaling is driven by binding to a PD-1 ligand (such as B7-H1 or B7-DC) in close proximity to a peptide antigen presented by major histocompatibility complex (MHC) (see, for example, Freeman, Proc Natl Acad Sci U. S. A, 105:10275-10276 (2008)). Therefore, proteins, antibodies or small molecules that prevent co-ligation of PD-1 and TCR on the T cell membrane are also useful PD-1 antagonists.

In some embodiments, the PD-1 receptor antagonists are small molecule antagonists or antibodies that reduce or interfere with PD-1 receptor signal transduction by binding to ligands of PD-1 or to PD-1 itself, especially where co-ligation of PD-1 with TCR does not follow such binding, thereby not triggering inhibitory signal transduction through the PD-1 receptor.

Other PD-1 antagonists contemplated by the methods of this invention include antibodies that bind to PD-1 or ligands of PD-1, and other antibodies.

Suitable anti-PD-1 antibodies include, but are not limited to, those described in the following U.S. Pat. Nos. 7,332,582, 7,488,802, 7,521,051, 7,524,498, 7,563,869, 7,981,416, 8,088,905, 8,287,856, 8,580,247, 8,728,474, 8,779,105, 9,067,999, 9,073,994, 9,084,776, 9,205,148, 9,358,289, 9,387,247, 9,492,539, 9,492,540, all of which are incorporated by reference in their entireties. See also Berger et al., Clin Cancer Res, 14:30443051 (2008).

Exemplary anti-B7-H1 (also referred to as anti-PD-L1) antibodies include, but are not limited to, those described in the following U.S. Pat. Nos. 8,383,796, 9,102,725, 9,273,135, 9,393,301, and 9,580,507 all of which are specifically incorporated by reference herein in their entirety.

For anti-B7-DC (also referred to as anti-PD-L2) antibodies see U.S. Pat. Nos. 7,411,051, 7,052,694, 7,390,888, 8,188,238, and 9,255,147 all of which are specifically incorporated by reference herein in their entirety.

Other exemplary PD-1 receptor antagonists include, but are not limited to B7-DC polypeptides, including homologs and variants of these, as well as active fragments of any of the foregoing, and fusion proteins that incorporate any of these. In some embodiments, the fusion protein includes the soluble portion of B7-DC coupled to the Fc portion of an antibody, such as human IgG, and does not incorporate all or part of the transmembrane portion of human B7-DC.

The PD-1 antagonist can also be a fragment of a mammalian B7-H1, for example from mouse or primate, such as a human, wherein the fragment binds to and blocks PD-1 but does not result in inhibitory signal transduction through PD-1. The fragments can also be part of a fusion protein, for example an Ig fusion protein.

Other useful polypeptides PD-1 antagonists include those that bind to the ligands of the PD-1 receptor. These include the PD-1 receptor protein, or soluble fragments thereof, which can bind to the PD-1 ligands, such as B7-H1 or B7-DC, and prevent binding to the endogenous PD-1 receptor, thereby preventing inhibitory signal transduction. B7-H1 has also been shown to bind the protein B7.1 (Butte et al., Immunity, Vol. 27, pp. 111-122, (2007)). Such fragments also include the soluble ECD portion of the PD-1 protein that includes mutations, such as the A99L mutation, that increases binding to the natural ligands (Molnar et al., PNAS, 105:10483-10488 (2008)). B7-1 or soluble fragments thereof, which can bind to the B7-H1 ligand and prevent binding to the endogenous PD-1 receptor, thereby preventing inhibitory signal transduction, are also useful.

PD-1 and B7-H1 anti-sense nucleic acids, both DNA and RNA, as well as siRNA molecules can also be PD-1 antagonists. Such anti-sense molecules prevent expression of PD-1 on T cells as well as production of T cell ligands, such as B7-H1, PD-L1 and/or PD-L2. For example, siRNA (for example, of about 21 nucleotides in length, which is specific for the gene encoding PD-1, or encoding a PD-1 ligand, and which oligonucleotides can be readily purchased commercially) complexed with carriers, such as polyethyleneimine (see Cubillos-Ruiz et al., J. Clin. Invest. 119(8): 2231-2244 (2009), are readily taken up by cells that express PD-1 as well as ligands of PD-1 and reduce expression of these receptors and ligands to achieve a decrease in inhibitory signal transduction in T cells, thereby activating T cells.

2. CTLA4 Antagonists

Other molecules useful in mediating the effects of T cells in an immune response are also contemplated as additional therapeutic agents. In some embodiments, the molecule is an antagonist of CTLA4, for example an antagonistic anti-CTLA4 antibody. An example of an anti-CTLA4 antibody contemplated for use in the methods of the invention includes an antibody as described in PCT/US2006/043690 (Fischkoff et al., WO/2007/056539).

Dosages for anti-PD-1, anti-B7-H1, and anti-CTLA4 antibody, are known in the art and can be in the range of, for example, 0.1 to 100 mg/kg, or with shorter ranges of 1 to 50 mg/kg, or 10 to 20 mg/kg. An appropriate dose for a human subject can be between 5 and 15 mg/kg, with 10 mg/kg of antibody (for example, human anti-PD-1 antibody) being a specific embodiment.

Specific examples of an anti-CTLA4 antibody useful in the methods of the invention are Ipilimumab, a human anti-CTLA4 antibody, administered at a dose of, for example, about 10 mg/kg, and Tremelimumab a human anti-CTLA4 antibody, administered at a dose of, for example, about 15 mg/kg. See also Sammartino, et al., Clinical Kidney Journal, 3(2):135-137 (2010), published online December 2009.

In other embodiments, the antagonist is a small molecule. A series of small organic compounds have been shown to bind to the B7-1 ligand to prevent binding to CTLA4 (see Erbe et al., J Biol Chem, 277:7363-7368 (2002). Such small organics could be administered alone or together with an anti-CTLA4 antibody to reduce inhibitory signal transduction of T cells.

3. Potentiating Agents

In some embodiments, additional therapeutic agents include a potentiating agent. The potentiating agent acts to increase efficacy the immune response up-regulator, possibly by more than one mechanism, although the precise mechanism of action is not essential to the broad practice of the present invention.

In some embodiments, the potentiating agent is cyclophosphamide. Cyclophosphamide (CTX, Cytoxan®, or Neosar®) is an oxazahosphorine drug and analogs include ifosfamide (IFO, Ifex), perfosfamide, trophosphamide (trofosfamide; Ixoten), and pharmaceutically acceptable salts, solvates, prodrugs and metabolites thereof (US patent application 20070202077 which is incorporated in its entirety). Ifosfamide (MITOXANA®) is a structural analog of cyclophosphamide and its mechanism of action is considered to be identical or substantially similar to that of cyclophosphamide. Perfosfamide (4-hydroperoxycyclophosphamide) and trophosphamide are also alkylating agents, which are structurally related to cyclophosphamide. For example, perfosfamide alkylates DNA, thereby inhibiting DNA replication and RNA and protein synthesis. New oxazaphosphorines derivatives have been designed and evaluated with an attempt to improve the selectivity and response with reduced host toxicity (Liang J, et al., “Design of New Oxazaphosphorine Anticancer Drugs”, Curr Pharm Des, 13(9):963-78 Review 2007). These include mafosfamide (NSC 345842), glufosfamide (D19575, beta-D-glucosylisophosphoramide mustard), S-(−)-bromofosfamide (CBM-11), NSC 612567 (aldophosphamide perhydrothiazine) and NSC 613060 (aldophosphamide thiazolidine). Mafosfamide is an oxazaphosphorine analog that is a chemically stable 4-thioethane sulfonic acid salt of 4-hydroxy-CPA. Glufosfamide is IFO derivative in which the isophosphoramide mustard, the alkylating metabolite of IFO, is glycosidically linked to a beta-D-glucose molecule. Additional cyclophosphamide analogs are described in U.S. Pat. No. 5,190,929 entitled “Cyclophosphamide analogs useful as anti-tumor agents” which is incorporated herein by reference in its entirety.

While CTX itself is nontoxic, some of its metabolites are cytotoxic alkylating agents that induce DNA crosslinking and, at higher doses, strand breaks. Many cells are resistant to CTX because they express high levels of the detoxifying enzyme aldehyde dehydrogenase (ALDH). CTX targets proliferating lymphocytes, as lymphocytes (but not hematopoietic stem cells) express only low levels of ALDH, and cycling cells are most sensitive to DNA alkylation agents.

Low doses of CTX (<200 mg/kg) can have immune stimulatory effects, including stimulation of anti-tumor immune responses in humans and mouse models of cancer (Brode & Cooke Crit Rev, Immunol, 28:109-126 (2008)). These low doses are sub-therapeutic and do not have a direct anti-tumor activity. In contrast, high doses of CTX inhibit the anti-tumor response. Several mechanisms may explain the role of CTX in potentiation of anti-tumor immune response: (a) depletion of CD4+CD25+FoxP3+ Treg (and specifically proliferating Treg, which may be especially suppressive), (b) depletion of B lymphocytes; (c) induction of nitric oxide (NO), resulting in suppression of tumor cell growth; (d) mobilization and expansion of CD11b+Gr-1+ MDSC. These primary effects have numerous secondary effects; for example following Treg depletion macrophages produce more IFN-γ and less IL-10. CTX has also been shown to induce type I IFN expression and promote homeostatic proliferation of lymphocytes.

Treg depletion is most often cited as the mechanism by which CTX potentiates the anti-tumor immune response. This conclusion is based in part by the results of adoptive transfer experiments. In the AB1-HA tumor model, CTX treatment at Day 9 gives a 75% cure rate. Transfer of purified Treg at Day 12 almost completely inhibited the CTX response (van der Most et al., Cancer Immunol. Immunother, 58:1219-1228 (2009). A similar result was observed in the HHD2 tumor model: adoptive transfer of CD4+CD25+ Treg after CTX pretreatment eliminated therapeutic response to vaccine (Taieb, J., J Immunol, 176:2722-2729 (2006)).

Numerous human clinical trials have demonstrated that low dose CTX is a safe, well-tolerated, and effective agent for promoting anti-tumor immune responses (Bas, & Mastrangelo Cancer Immunol, Immunother, 47:1-12 (1998)).

The optimal dose for CTX to potentiate an anti-tumor immune response, is one that lowers overall T cell counts by lowering Treg levels below the normal range but is subtherapeutic (see Machiels et al., Cancer Res, 61:3689-3697 (2001)).

In human clinical trials where CTX has been used as an immunopotentiating agent, a dose of 300 mg/m² has usually been used. For an average male (6 ft., 170 pound (78 kg) with a body surface area of 1.98 m²), 300 mg/m² is 8 mg/kg, or 624 mg of total protein. In mouse models of cancer, efficacy has been seen at doses ranging from 15-150 mg/kg, which relates to 0.45-4.5 mg of total protein in a 30 g mouse (Machiels et al., Cancer Res, 61:3689-3697 (2001), Hengst et al., Cancer Res, 41:2163-2167 (1981), Hengst, Cancer Res, 40:2135-2141 (1980)).

For larger mammals, such as a primate, such as a human, patient, such mg/m² doses may be used but unit doses administered over a finite time interval may also be used. Such unit doses may be administered on a daily basis for a finite time period, such as up to 3 days, or up to 5 days, or up to 7 days, or up to 10 days, or up to 15 days or up to 20 days or up to 25 days, are all specifically contemplated by the invention. The same regimen may be applied for the other potentiating agents recited herein.

In other embodiments, the potentiating agent is an agent that reduces activity and/or number of regulatory T lymphocytes (T-regs), such as Sunitinib (SUTENT®), anti-TGFβ or Imatinib (GLEEVAC®). The recited treatment regimen may also include administering an adjuvant.

Useful potentiating agents also include mitosis inhibitors, such as paclitaxol, aromatase inhibitors (e.g. Letrozole) and angiogenesis inhibitors (VEGF inhibitors e.g., Avastin, VEGF-Trap) (see, for example, Li et al., Clin Cancer Res, November 15; 12(22):6808-16 (2006.)), anthracyclines, oxaliplatin, doxorubicin, TLR4 antagonists, and IL-18 antagonists.

4. Antimicrobials

For example, KLRG1-binding or KLRG1 ligand-binding molecules can be used in a preventive or prophylactic role in the treatment and prevention of disease as discussed above, and also in the context of severe trauma injuries like a major burn, open bone fracture, accidental amputation or other wounds. Therefore, the KLRG1-binding or KLRG1 ligand-binding molecules can be administered to the subject in combination with an antimicrobial such as an antibiotic, an antifungal, an antiviral, an antiparasitics, or essential oil.

In some embodiments, the subject is administered the KLRG1-binding molecules and/or the antimicrobial at time of admission to the hospital to prevent further bacterial, fungal or viral complications. The antibiotic can target pathogens and the KLRG1-binding or KLRG1 ligand-binding molecule can stimulate the immune system to provide an enhanced response to treat or prevent further infection or disease.

### V. Diagnostic Methods

The disclosed KLRG1-binding molecules, particularly antibodies and their antigen-binding fragments, can be used for diagnostic purposes, such as to detect, diagnose, or monitor diseases, disorders or infections associated with KLRG1 expression, or to determine or assist in the determination or identification of suitable patient populations or profiles. Any of the methods can be coupled with a method of treating the subject, for example, by administering the subject an effective amount of one or more therapeutic KLRG1-binding molecules.

The detection or diagnosis of a disease, disorder or infection, including, but not limited to, cancer can include: (a) assaying the expression of KLRG1 or derivatives thereof in cells, serum, plasma, blood or in a tissue sample (e.g., a tumor sample) of a subject using one or more antibodies (or fragments thereof) that immunospecifically bind to such antigens; and (b) comparing the level of the antigen with a control level, e.g., levels in normal tissue samples, whereby an increase in the assayed level of antigen compared to the control level of the antigen is indicative of the disease, disorder or infection. Such antibodies and fragments can be employed in immunoassays, such as the enzyme linked immunosorbent assay (ELISA), the radioimmunoassay (RIA) and fluorescence-activated cell sorting (FACS).

In some embodiments, the antibodies or fragments are used for IHC analysis in cells of an in vitro or in situ tissue sample or in vivo. Thus, the antibodies and fragments can be used in the detection and diagnosis of a disease, disorder, or infection in a human. In one embodiment, such diagnosis includes: a) administering to a subject (for example, parenterally, subcutaneously, or intraperitoneally) an effective amount of such labeled antibody or antigen-binding fragment; b) waiting for a time interval following the administration for permitting the labeled molecule to preferentially concentrate at sites in the subject where KLRG1 is expressed (and for unbound labeled molecule to be cleared to background level); c) determining background level; and d) detecting the labeled antibody in the subject, such that localized detection of labeled antibody above or below the background level indicates that the subject has the disease, disorder, or infection and/or shows the location and relative expression level of KLRG1+ tissue. In accordance with this embodiment, the antibody can be labeled with an imaging moiety which is detectable in vivo using an imaging system known to one of skill in the art. Background level can be determined by various methods including, comparing the amount of labeled molecule detected to a standard value previously determined for a particular system.

Other methods include, for example, monitoring the progression of a disease, disorder or infection, by (a) assaying the expression of KLRG1 in cells or in a tissue sample of a subject obtained at a first time point and later time point using a KLRG1-binding molecule and (b) comparing the level of expression of KLRG1 in the cells or in the tissue sample of the subject at the first and later times points, wherein an increase in the assayed level of KLRG1 at the later time point compared to the first time point is indicative of the progression of disease, disorder or infection.

A method for monitoring a response to a treatment, can include, (a) assaying the expression of KLRG1 in cells or in a tissue sample of a subject prior and after the treatment using a KLRG1-binding molecule; and (b) comparing the level of KLRG1 over time, whereby a decrease in the assayed level of KLRG1 after treatment compared to the level of KLRG1 prior to treatment is indicative of a favorable response to the treatment.

It will be understood in the art that the size of the subject and the imaging system used will determine the quantity of imaging moiety needed to produce diagnostic images.

Depending on several variables, including the type of label used and the mode of administration, the time interval following the administration for permitting the labeled molecule to preferentially concentrate at sites in the subject and for unbound labeled molecule to be cleared to background level is 6 to 48 hours or 6 to 24 hours or 6 to 12 hours. In another embodiment the time interval following administration is 5 to 20 days or 5 to 10 days.

In one embodiment, monitoring of a disease, disorder or infection is carried out by repeating the method for diagnosing the disease, disorder or infection, for example, one month after initial diagnosis, six months after initial diagnosis, one year after initial diagnosis, etc.

Presence of the labeled molecule can be detected in the subject using methods known in the art for in vivo scanning. These methods depend upon the type of label used. Skilled artisans will be able to determine the appropriate method for detecting a particular label. Methods and devices that can be used in the diagnostic methods include, but are not limited to, computed tomography (CT), whole body scan such as position emission tomography (PET), magnetic resonance imaging (MRI), and sonography.

In a specific embodiment, the molecule is labeled with a radioisotope and is detected in the patient using a radiation responsive surgical instrument (Thurston et al., U.S. Pat. No. 5,441,050). In another embodiment, the molecule is labeled with a fluorescent compound and is detected in the patient using a fluorescence responsive scanning instrument. In another embodiment, the molecule is labeled with a positron emitting metal and is detected in the patient using positron emission-tomography. In yet another embodiment, the molecule is labeled with a paramagnetic label and is detected in a patient using magnetic resonance imaging (MRI).

### VI. Kits

The disclosed KLRG1-binding or KLRG1 ligand-binding molecules can be packaged in a hermetically sealed container, such as an ampoule or sachette, indicating the quantity. The molecules can be supplied as a dry sterilized lyophilized powder or water free concentrate in a hermetically sealed container and can be reconstituted, e.g., with water or saline to the appropriate concentration for administration to a subject. For example, the molecules can be supplied as a dry sterile lyophilized powder in a hermetically sealed container at a unit dosage of at least 5 mg, or at least 10 mg, at least 15 mg, at least 25 mg, at least 35 mg, at least 45 mg, at least 50 mg, or at least 75 mg. The lyophilized molecules can be stored at between 2 and 8° C. in their original container and are typically administered within 12 hours, or within 6 hours, or within 5 hours, or within 3 hours, or within 1 hour after being reconstituted.

In an alternative embodiment, molecules supplied in liquid form in a hermetically sealed container indicating the quantity and concentration. In some embodiments, the liquid form of the molecules supplied in a hermetically sealed container including at least 1 mg/ml, or at least 2.5 mg/ml, at least 5 mg/ml, at least 8 mg/ml, at least 10 mg/ml, at least 15 mg/ml, at least 25 mg/ml, at least 50 mg/ml, at least 100 mg/ml, at least 150 mg/ml, at least 200 mg/ml of the molecules.

Pharmaceutical packs and kits including one or more containers filled with KLRG1-binding or KLRG1 ligand-binding molecules are also provided. Additionally, one or more other prophylactic or therapeutic agents useful for the treatment of a disease can also be included in the pharmaceutical pack or kit. The pharmaceutical pack or kit can also include one or more containers filled with one or more of the ingredients of the disclosed pharmaceutical compositions. Optionally associated with such container(s) can be a notice in the form prescribed by a governmental agency regulating the manufacture, use or sale of pharmaceuticals or biological products, which notice reflects approval by the agency of manufacture, use or sale for human administration.

Kits designed for the above-described methods are also provided. Embodiments typically include one or more KLRG1-binding or KLRG1 ligand-binding molecules. In particular embodiments, a kit also includes one or more other prophylactic or therapeutic agents useful for the treatment of cancer, in one or more containers.

1.-164. (canceled)

165. An anti-KLRG1 antibody or antigen-binding fragment thereof comprising:
a light chain variable region having at least 50%, 60%, 70%, 80%, 85%, 90%, 95%, 99%, or more sequence identity to SEQ ID NO:147, and
a heavy chain viable region having at least 50%, 60%, 70%, 80%, 85%, 90%, 95%, 99%, or more sequence identity to SEQ ID NO:82,
wherein the antibody or antigen binding fragment thereof binds to KLRG1.

166. The antibody or antigen binding fragment thereof of claim 165, further comprising one or more constant domains from an immunoglobulin constant region (Fc).

167. The antibody of claim 165, wherein the antibody is humanized.

168. An anti-KLRG1 antibody or antigen-binding fragment thereof comprising:
a light chain variable region at least 50%, 60%, 70%, 80%, 85%, 90%, 95%, 99%, or more sequence identity to an amino acid sequence selected from the group consisting of SEQ ID Nos: 121, 122, 123, 124, and 125, and
a heavy chain viable region having at least 50%, 60%, 70%, 80%, 85%, 90%, 95%, 99%, or more sequence identity to an amino acid sequence selected from the group consisting of SEQ ID Nos: 132, 133, 134, 135, and 136,
wherein the antibody or antigen binding fragment thereof binds to KLRG1.

169. The antibody or antigen binding fragment thereof of claim 168, further comprising one or more constant domains from an immunoglobulin constant region (Fc).

170. The antibody or antigen binding fragment thereof of claim 169, wherein the constant domains are human constant domains.

171. The antibody or antigen binding fragment thereof of claim 170, wherein the human constant domains are IgA, IgD, IgE, IgG or IgM domains.

172. The antibody or antigen binding fragment thereof of claim 170, wherein human IgG constant domains are IgG1, IgG2, IgG3, or IgG4 domains, or mutated variants thereof having reduced or silenced FcR binding.

173. The antibody or antigen binding fragment thereof of claim 168, wherein the antibody or antigen binding fragment thereof is detectably labeled or comprises a conjugated toxin, drug, receptor, enzyme, receptor ligand.

174. The antibody or antigen binding fragment thereof of claim 168, wherein the antibody is a monoclonal antibody, a human antibody, a chimeric antibody, a humanized antibody, or a single chain antibody.

175. The antibody or antigen binding fragment thereof of claim 168, wherein the antibody is a monospecific, bispecific, trispecific, or multispecific antibody.

176. A pharmaceutical composition comprising the antibody or antigen binding fragment thereof of claim 168 and a pharmaceutically acceptable excipient.

177. A method of treating a subject in need thereof comprising administering to the subject an effective amount of the pharmaceutical composition of claim 176 to the subject in an amount effective to inhibit, reduce or block signal transduction through KLRG1 on immune cells.

178. The method of claim 177, wherein the subject has cancer or an infectious disease.

179. The method of claim 177, further comprising administering to the subject a second therapeutic agent.

180. A nucleic acid encoding the antibody of claim 168.


SEQUENCE LISTING (147 sequences)

SEQ ID NO: 1 - protein, 136 aa, Homo sapiens [sequence omitted]

SEQ ID NO: 2 - protein, 132 aa, Mus musculus
QRILCCGSKDSTCSHCPSCPILWTRNGSHCYYFSMEKKDWNSSLKFCADKGSHLLTFPDNQGVKLFGEYLGQDFYWIGLRNIDGWRWEGGPALSLRILTNSLIQRCGAIHRNGLQASSCEVALQWICKKVLY

SEQ ID NO: 3 - protein, 38 aa, Homo sapiens
MTDSVIYSMLELPTATQAQNDYGPQQKSSSSRPSCSCL

SEQ ID NO: 4 - protein, 107 aa, Artificial Sequence
  Synthetic
DIVMTQSHKFMSTSVGDRVSITCKASQDVATAVDWYQQKPGQSPKLLIYWASTRHTGVPDRFTGSGSGTDFTLTISNVQSEDLADYFCQQYSSYPLTFGTGTKLELK

SEQ ID NO: 5 - protein, 11 aa, Artificial Sequence
  Synthetic
KASQDVATAVD

SEQ ID NO: 6 - protein, 7 aa, Artificial Sequence
  Synthetic
WASTRHT

SEQ ID NO: 7 - protein, 9 aa, Artificial Sequence
  Synthetic
QQYSSYPLT

SEQ ID NO: 8 - DNA, 321 bp, Artificial Sequence
  Synthetic
gacattgtgatgacccagtctcacaaattcatgtccacatcagtaggagacagggtcagcatcacctgcaaggccagtcaggatgtggctactgctgtagactggtatcaacagaaaccagggcaatctcctaaactactgatttattgggcatctacccggcacactggagtccctgatcgcttcacaggcagtggatctgggacagatttcactctcaccattagcaatgtgcagtctgaagacttggcagattatttctgtcagcaatatagcagctatcctctcacgttcggtactgggaccaagctggagctgaaa

SEQ ID NO: 9 - protein, 120 aa, Artificial Sequence
  Synthetic
EIQLQQSGAELVKPGASVKISCKASGYSFTGYNMNWVRQSHGKSLEWIGNINPYFGSSIYNQRFKGKATLTVDKSSSTAYMQLNSLTSEDSAVYYCAKNYDYERDAMDKWGQGTSVTVSS

SEQ ID NO: 10 - protein, 5 aa, Artificial Sequence
  Synthetic
GYNMN

SEQ ID NO: 11 - protein, 17 aa, Artificial Sequence
  Synthetic
NINPYFGSSIYNQRFKG

SEQ ID NO: 12 - protein, 11 aa, Artificial Sequence
  Synthetic
NYDYERDAMDK

SEQ ID NO: 13 - DNA, 360 bp, Artificial Sequence
  Synthetic
gagatccagctgcagcagtctggagctgagctggtgaagcctggggcttcagtgaagatatcctgcaaggcttctggttactcattcactggctacaacatgaactgggtgaggcagagccatggaaagagccttgagtggattggaaatattaatccttactttggtagttctatctacaatcagaggtttaagggcaaggccacattgactgtagacaaatcctccagcacagcctacatgcagctcaacagcctgacatctgaggactcagcagtctattactgtgcgaaaaactatgattacgagagagatgctatggacaaatggggtcaaggaacctcagtcaccgtctcctca

SEQ ID NO: 14 - protein, 107 aa, Artificial Sequence
  Synthetic
DIVLTQSPATLSVTLGDSVSLSCRASQSISNNLHWYQQKSHESPRLLIKYASQSISGIPSRFSGSGSGTDFTLSINSVETEDFGMYFCQQSNSWPHTFGGGTKLEIK

SEQ ID NO: 15 - protein, 11 aa, Artificial Sequence
  Synthetic
RASQSISNNLH

SEQ ID NO: 16 - protein, 7 aa, Artificial Sequence
  Synthetic
YASQSIS

SEQ ID NO: 17 - protein, 9 aa, Artificial Sequence
  Synthetic
QQSNSWPHT

SEQ ID NO: 18 - DNA, 321 bp, Artificial Sequence
  Synthetic
gatattgtgctaactcagtctccagccaccctgtctgtgactctaggagatagcgtcagtctttcctgcagggccagccaaagtattagcaacaacctacactggtatcaacaaaaatcacatgagtctccaaggcttctcattaagtatgcttcccagtccatctctgggatcccctccaggttcagtggcagtggatcagggacagatttcactctcagtatcaacagtgtggagactgaagattttggaatgtatttctgtcaacagagtaacagctggcctcatacgttcggaggggggaccaagctggaaataaaa

SEQ ID NO: 19 - protein, 113 aa, Artificial Sequence
  Synthetic
EVQLQQSGPELVKPGASVKMSCKASGYTFTGYVMHWVKQKPGQGLEWIGYINPYNDGTRYSEKFKGKATLTSDKSSSTAYMELSSLTSEDSAVYYCARGRDYWGQGTTLTVSS

SEQ ID NO: 20 - protein, 5 aa, Artificial Sequence
  Synthetic
GYVMH

SEQ ID NO: 21 - protein, 17 aa, Artificial Sequence
  Synthetic
YINPYNDGTRYSEKFKG

SEQ ID NO: 22 - protein, 4 aa, Artificial Sequence
  Synthetic
GRDY

SEQ ID NO: 23 - DNA, 339 bp, Artificial Sequence
  Synthetic
gaggtccagctgcagcagtctggacctgaactggtaaagcctggggcttcagtgaagatgtcctgcaaggcttctggatacacattcactggctatgttatgcactgggtgaagcagaagcctgggcagggccttgagtggattggatatattaatccttacaatgatggtactaggtacagtgagaagttcaaaggcaaggccacactgacttcagacaaatcctccagcacagcctacatggagctcagcagcctgacctctgaggactctgcggtctattactgtgcaagagggcgggactactggggccaaggcaccactctcacagtctcctca

SEQ ID NO: 24 - protein, 107 aa, Artificial Sequence
  Synthetic
DIVMTQSQKFMSTSVGDRVSITCKASQNVGSTVVWYQQKPGQSPKLLIYSASNRYTGVPDRFTGNGSGTDFTLTISNMQSEDLADYFCQQCSSYPLTFGAGTKLELK

SEQ ID NO: 25 - protein, 11 aa, Artificial Sequence
  Synthetic
KASQNVGSTVV

SEQ ID NO: 26 - protein, 7 aa, Artificial Sequence
  Synthetic
SASNRYT

SEQ ID NO: 27 - protein, 9 aa, Artificial Sequence
  Synthetic
QQCSSYPLT

SEQ ID NO: 28 - DNA, 321 bp, Artificial Sequence
  Synthetic
gacattgtgatgacccagtctcaaaaattcatgtccacatcagtaggagacagggtcagcatcacctgcaaggccagtcagaatgtgggtagtactgtagtctggtatcaacagaaaccaggacaatctcctaaactactgatttactcggcatccaatcggtacactggagtccctgatcgcttcacaggcaatggatctgggacagatttcactctcaccatcagcaatatgcagtctgaagacctggcagattatttctgccagcaatgtagcagctatcctctcacgttcggtgctgggaccaagctggagctgaaa

SEQ ID NO: 29 - protein, 120 aa, Artificial Sequence
  Synthetic
QVQLQQSGAELVRPGASVKISCKAFGYTFTNHFINWVKQRPGQGLDWIGYINPYNGYTNYNQKFKGKATLTVDKSSSTACMELSSLTSEDSAVYYCAISYDGYYERFAYWGQGTLVTVSA

SEQ ID NO: 30 - protein, 5 aa, Artificial Sequence
  Synthetic
NHFIN

SEQ ID NO: 31 - protein, 16 aa, Artificial Sequence
  Synthetic
YINPYNGYTNYNQKFK

SEQ ID NO: 32 - protein, 11 aa, Artificial Sequence
  Synthetic
SYDGYYERFAY

SEQ ID NO: 33 - DNA, 360 bp, Artificial Sequence
  Synthetic
caggtccagctgcagcagtctggggctgagctggtgaggcctggggcctcagtgaagatttcctgcaaggcttttggctacaccttcacaaaccattttataaactgggtgaagcagaggcctggacagggcctggactggattggatatattaatccttataatggttatacaaactacaaccagaagttcaagggcaaggccacattgactgtagacaaatcctccagcacagcctgtatggagcttagcagcctgacatctgaggactctgcagtctattactgtgccatatcctatgatggttactacgagaggtttgcttactggggccaagggactctggtcactgtctctgca

SEQ ID NO: 34 - protein, 108 aa, Artificial Sequence
  Synthetic
ENVLTQSPAIMSASPGEKVTMTCRASSSVSSNYLHWYQQKSGASPKIWIYSTSNLASAVPARFSGSGSGTSYSLTISSVEAEDAATYYCQQYSGYPLTFGGGTKLEIK

SEQ ID NO: 35 - protein, 12 aa, Artificial Sequence
  Synthetic
RASSSVSSNYLH

SEQ ID NO: 36 - protein, 7 aa, Artificial Sequence
  Synthetic
STSNLAS

SEQ ID NO: 37 - protein, 9 aa, Artificial Sequence
  Synthetic
QQYSGYPLT

SEQ ID NO: 38 - DNA, 324 bp, Artificial Sequence
  Synthetic
gaaaatgtgctcacccagtctccagcaatcatgtctgcatctccaggggaaaaggtcaccatgacctgcagggccagctcaagtgttagttccaattacttgcactggtaccagcagaagtcaggtgcctcccccaaaatctggatttatagcacatccaatctggcttctgcagtccctgcgcgcttcagtggcagtgggtctgggacctcttactctctcacaatcagcagtgtggaggctgaagatgctgccacttattactgccagcagtacagtggttacccactgacgttcggtggaggcaccaagttggaaatcaaa

SEQ ID NO: 39 - protein, 118 aa, Artificial Sequence
  Synthetic
QVQLQQPGAEFVKPGASVKLSCKASGYTFTSYWIHWMKQRPGQGLEWIGNIYPGRSNNNYNEKFKNRATLTVDTSSSTAYMQFRSLTSDDSAVYYCARDATVEPLPYWGQGTLVTVSA

SEQ ID NO: 40 - protein, 5 aa, Artificial Sequence
  Synthetic
SYWIH

SEQ ID NO: 41 - protein, 17 aa, Artificial Sequence
  Synthetic
NIYPGRSNNNYNEKFKN

SEQ ID NO: 42 - protein, 9 aa, Artificial Sequence
  Synthetic
DATVEPLPY

SEQ ID NO: 43 - DNA, 354 bp, Artificial Sequence
  Synthetic
caggtccaactgcagcagcctggtgctgagtttgtgaagcctggggcctcagtgaagctgtcgtgcaaggcttctggctacactttcaccagctactggatacattggatgaagcagaggcctggacaaggccttgagtggattggaaatatttatcctggtagaagtaataataactacaatgagaagttcaagaacagggccacactgactgtagacacatcctccagcacagcctacatgcagttcagaagcctgacatctgacgactctgcggtctattattgtgcaagagatgctacggtggagcctcttccttactggggccaagggactctggtcactgtctctgca

SEQ ID NO: 44 - protein, 108 aa, Artificial Sequence
  Synthetic
EIVLTQSPALMAASPGEKVTITCSVSSSISSSNLHWYQQKSETSPKPWIYGTSNLASGVPVRFSGSGSGTSYSLTISSMEAEDAATYYCQQWSSYPLTFGAGTKLELK

SEQ ID NO: 45 - protein, 12 aa, Artificial Sequence
  Synthetic
SVSSSISSSNLH

SEQ ID NO: 46 - protein, 7 aa, Artificial Sequence
  Synthetic
GTSNLAS

SEQ ID NO: 47 - protein, 9 aa, Artificial Sequence
  Synthetic
QQWSSYPLT

SEQ ID NO: 48 - DNA, 324 bp, Artificial Sequence
  Synthetic
gaaattgtgctcacccagtctccagcactcatggctgcatctccaggggagaaggtcaccatcacctgcagtgtcagctcaagtataagttccagcaacttgcactggtaccagcagaagtcagaaacctcccccaaaccctggatttatggcacatccaacctggcttctggagtccctgttcgcttcagtggcagtggatctgggacctcttattctctcacaatcagcagcatggaggctgaagatgctgccacttattactgtcaacagtggagtagttacccactcacgttcggtgctgggaccaagctggagctgaaa

SEQ ID NO: 49 - protein, 123 aa, Artificial Sequence
  Synthetic
QVQLQQPGAELVKPGASVKLSCKASGYTFTSYWINWVKQRPGQGLEWIGNIYPGSSSTNYNEKFKYKATLTVDTSSSTANMQLSSLTSDDSAVYYCARGRLLRLRRGGYFDYWGQGTTLTVSS

SEQ ID NO: 50 - protein, 5 aa, Artificial Sequence
  Synthetic
SYWIN

SEQ ID NO: 51 - protein, 17 aa, Artificial Sequence
  Synthetic
NIYPGSSSTNYNEKFKY

SEQ ID NO: 52 - protein, 14 aa, Artificial Sequence
  Synthetic
GRLLRLRRGGYFDY

SEQ ID NO: 53 - DNA, 369 bp, Artificial Sequence
  Synthetic
caggtccaactgcagcagcctggtgctgagcttgtgaagcctggggcctcagtgaagctgtcctgcaaggcttctggctacactttcaccagctactggataaactgggtgaagcagaggcctggacaaggccttgagtggattggaaatatttatcctggtagtagtagtactaattacaatgagaagttcaagtacaaggccacactgactgtagacacatcctccagtacagccaacatgcagctcagcagcctgacatctgacgactctgcggtctattattgtgcaagaggtcgtttattacggctaagacgagggggctactttgactactggggccaaggcaccactctcacagtctcctca

SEQ ID NO: 54 - DNA, 321 bp, Artificial Sequence; identical to SEQ ID NO: 28
  Synthetic

SEQ ID NO: 55 - protein, 120 aa, Artificial Sequence
  Synthetic
QVQLQQSGAELVRPGASVKISCKAFGYTFTNHFINWVKQRPGQGLDWIGYINPYNGYTNYNQKFKGKATLTVDKSSSTAYMELSSLTSEDSAVYYCAISYDGYYERFAYWGQGTLVTVSA

SEQ ID NO: 56 - DNA, 360 bp, Artificial Sequence
  Synthetic
caggtccagctgcagcagtctggggctgagctggtgaggcctggggcctcagtgaagatttcctgcaaggcttttggctacaccttcacaaaccattttataaactgggtgaagcagaggcctggacagggcctggactggattggatatattaatccttataatggttatacaaactacaaccagaagttcaagggcaaggccacattgactgtagacaaatcctccagcacagcctatatggagcttagcagcctgacatctgaggactctgcagtctattactgtgccatatcctatgatggttactacgagaggtttgcttactggggccaagggactctggtcactgtctctgca

SEQ ID NO: 57 - protein, 107 aa, Artificial Sequence
  Synthetic
DIQMTQSPSSLSASLGERVSLTCRASQDIGSSLNWLQQEPDGTIKRLIYATSSLDSGVPKRFSGSRSGSDYSLTISSLESEDFVDYYCLQYASSPPTFGGGTKLEIK

SEQ ID NO: 58 - protein, 11 aa, Artificial Sequence
  Synthetic
RASQDIGSSLN

SEQ ID NO: 59 - protein, 7 aa, Artificial Sequence
  Synthetic
ATSSLDS

SEQ ID NO: 60 - protein, 9 aa, Artificial Sequence
  Synthetic
LQYASSPPT

SEQ ID NO: 61 - DNA, 320 bp, Artificial Sequence
  Synthetic
gacatccagatgacccagtctccatcctccttatctgcctctctgggagaaagagtcagtctcacttgtcgggcaagtcaggacattggtagtagcttaaactggcttcagcaggaaccagatggaactattaaacgcctgatctacgccacatccagtttagattctggtgtccccaaaaggttcagtggcagtaggtctgggtcagattattctctcaccatcagcagccttgagtctgaagattttgtagactattactgtctccaatatgctagttctcctccgacgttcggtgggggcaccaaactggaaatcaa

SEQ ID NO: 62 - protein, 115 aa, Artificial Sequence
  Synthetic
EVQLVESGGGLVKPGGTLKLSCAAAGFTFTRYDMSWVRQIPAKRLEWIATISGGGGYSFYPDSVKGRFTISRDNAKNTLYLQMSSLRSDDTAVYYCVREDAMDYWGQGTSVTVSS

SEQ ID NO: 63 - protein, 5 aa, Artificial Sequence
  Synthetic
RYDMS

SEQ ID NO: 64 - protein, 17 aa, Artificial Sequence
  Synthetic
TISGGGGYSFYPDSVKG

SEQ ID NO: 65 - protein, 6 aa, Artificial Sequence
  Synthetic
EDAMDY

SEQ ID NO: 66 - DNA, 345 bp, Artificial Sequence
  Synthetic
gaagtgcagctggtggagtctgggggaggcttagtgaagcctggagggaccctgaaactctcctgtgcagccgctggattcactttcactagatatgacatgtcttgggttcgtcagattccggcgaagaggctggagtggatcgcaaccattagtggtggtggtggttacagcttctatccagacagtgtgaagggccgattcaccatctccagagacaatgccaagaacaccctgtatctgcaaatgagcagtctgaggtctgatgacacagccgtgtattactgtgtaagggaggatgctatggactattggggtcaaggaacgtcagtcaccgtctcctca

SEQ ID NO: 67 - protein, 107 aa, Artificial Sequence
  Synthetic
DIQMTQSPSSLSASLGERISLTCRASQDIYGSLNWFQQKPDGTIKLLIYGTSSLDSGVPKRFSGSRSGSDYSLTISSLESEDFADYYCLQYASFPLTFGAGTKLELK

SEQ ID NO: 68 - protein, 11 aa, Artificial Sequence
  Synthetic
RASQDIYGSLN

SEQ ID NO: 69 - protein, 7 aa, Artificial Sequence
  Synthetic
GTSSLDS

SEQ ID NO: 70 - protein, 9 aa, Artificial Sequence
  Synthetic
LQYASFPLT

SEQ ID NO: 71 - DNA, 321 bp, Artificial Sequence
  Synthetic
gacatccagatgacccagtctccatcctccttatctgcctctctgggagaaagaatcagtctcacttgccgggcaagtcaggacatttatggtagcttaaactggtttcagcagaaaccagatggaactattaaactcctgatctacggcacatccagtttagattctggtgtccccaaaaggttcagtggcagtaggtctgggtcagattattctctcaccatcagcagccttgagtctgaagattttgcagactattactgtctacaatatgctagttttccgctcacgttcggtgctgggaccaagctggagctgaaa

SEQ ID NO: 72 - protein, 121 aa, Artificial Sequence
  Synthetic
QVQLQQPGAEMVRPGASVKLSCKTSGYTFTSYWMHWVKQRPGQGLEWIGKIDPSDSETHYNQKFKDKATLTVDKSSSTAYIQLNSLTSEDSAVYYCARGRLLRLRDWFPYWGQGTLVTVSA

SEQ ID NO: 73 - protein, 5 aa, Artificial Sequence
  Synthetic
SYWMH

SEQ ID NO: 74 - protein, 17 aa, Artificial Sequence
  Synthetic
KIDPSDSETHYNQKFKD

SEQ ID NO: 75 - protein, 12 aa, Artificial Sequence
  Synthetic
GRLLRLRDWFPY

SEQ ID NO: 76 - DNA, 363 bp, Artificial Sequence
  Synthetic
caggtccagctgcagcagcctggggctgagatggtgaggcctggggcttcagtgaagttgtcctgcaagacttctggctacaccttcaccagctactggatgcactgggtgaagcagaggcctggacaaggccttgagtggattggtaagattgatccttctgatagtgaaactcactacaatcaaaagttcaaggacaaggccacattgactgtcgacaaatcctccagcacagcctacatacagctcaacagcctgacatctgaagactctgcggtctattactgtgcaagaggaaggttactacggctacgtgactggtttccttactggggccaagggactctggtcactgtctctgca

SEQ ID NO: 77 - protein, 107 aa, Artificial Sequence
  Synthetic
DIQMTQSPASLSASVGETVTITCRASENIYSCLAWYQQKQGKSPQFLVHNAKTLAEGVPSRFSGSGSGTLFSLKINSLQPEDFGSYYCQYHYGIPFTFGSGTKLEIK

SEQ ID NO: 78 - protein, 11 aa, Artificial Sequence
  Synthetic
RASENIYSCLA

SEQ ID NO: 79 - protein, 7 aa, Artificial Sequence
  Synthetic
NAKTLAE

SEQ ID NO: 80 - protein, 9 aa, Artificial Sequence
  Synthetic
QYHYGIPFT

SEQ ID NO: 81 - DNA, 321 bp, Artificial Sequence
  Synthetic
gacatccagatgactcagtctccagcctccctatctgcatctgtgggagaaactgtcaccatcacatgtcgagcaagcgagaatatttatagttgtttagcatggtatcagcagaaacagggaaaatctcctcagttcctggtccataatgcaaaaaccttagctgaaggtgtgccatcaaggttcagtggcagtggatcaggcacactattttctctgaagatcaacagcctgcagcctgaagattttgggagttattactgtcaatatcattatggcattccattcacgttcggctcggggacaaagttggaaataaaa

SEQ ID NO: 82 - protein, 120 aa, Artificial Sequence
  Synthetic
DVKLVESGGGLVKLGGSLKLSCAASGFTFSRYYMSWVRQTPEKRLEWVATISISGGNTYYPDTMKGRFTISRDSAKNTLYLQMSSLNSEDTAVYYCAREGGYGNLWFAYWGQGTLVTVSA

SEQ ID NO: 83 - protein, 5 aa, Artificial Sequence
  Synthetic
RYYMS

SEQ ID NO: 84 - protein, 17 aa, Artificial Sequence
  Synthetic
TISISGGNTYYPDTMKG

SEQ ID NO: 85 - protein, 11 aa, Artificial Sequence
  Synthetic
EGGYGNLWFAY

SEQ ID NO: 86 - DNA, 360 bp, Artificial Sequence
  Synthetic
gacgtgaagctcgtggagtctgggggaggcttagtgaagcttggagggtccctgaaactctcctgtgcagcctctggattcactttcagtcgctattacatgtcttgggttcgccagactccggagaagaggctggagtgggtcgcaaccattagtattagtggtggtaacacctactacccagacactatgaagggccgattcaccatctccagagacagtgccaagaacaccctgtacctgcaaatgagcagtctgaattctgaggacacagccgtgtattactgtgcaagagaaggggggtatggtaacctctggtttgcttactggggccaagggactctggtcactgtctctgca

SEQ ID NO: 87 - protein, 107 aa, Artificial Sequence
  Synthetic
DIQMTQSPSSLSASLGERVSLTCRASQDIGSSLNWLQQEPDGTIKRLIYATSSLDSGVPKRFSGSRSGSDYSLTISSLESEDFVDYYCLQYAGSPPTFGSGTKLELK

SEQ ID NO: 88 - protein, 9 aa, Artificial Sequence
  Synthetic
LQYAGSPPT

SEQ ID NO: 89 - DNA, 321 bp, Artificial Sequence
  Synthetic
gacatccagatgacccagtctccatcctccttatctgcctctctgggagaaagagtcagtctcacttgtcgggcaagtcaggacattggtagtagcttaaactggcttcagcaggaaccagatggaactattaaacgcctgatctacgccacatccagtttagattctggtgtccccaaaaggttcagtggcagtaggtctgggtcagattattctctcaccatcagcagccttgagtctgaagattttgtagactattactgtctacaatatgctggttctcctcccacgttcggttctgggaccaagctggagctgaaa

SEQ ID NO: 90 - protein, 122 aa, Artificial Sequence
  Synthetic
EVQLVESGGGLVKPGGSLKLSCAASGFTFSNYYMSWVRQTPAKRLEWVATISGGGGYTFYPDSLKGRFTISRDNAKNTLYLQMSSLRSEDTAMYYCARDQDYGTIYYAMDYWGQGTSVTVSS

SEQ ID NO: 91 - protein, 5 aa, Artificial Sequence
  Synthetic
NYYMS

SEQ ID NO: 92 - protein, 17 aa, Artificial Sequence
  Synthetic
TISGGGGYTFYPDSLKG

SEQ ID NO: 93 - protein, 13 aa, Artificial Sequence
  Synthetic
DQDYGTIYYAMDY

SEQ ID NO: 94 - DNA, 366 bp, Artificial Sequence
  Synthetic
gaagtgcagctggtggagtctgggggaggcttagtgaagcctggagggtccctgaaactctcctgtgcagcctctggattcactttcagtaactattacatgtcttgggttcgccagactccggcgaagaggctggagtgggtcgcaaccattagtggtggtggtggttacaccttctatccagacagtttgaagggccgattcaccatctccagagacaatgccaagaacaccctatacctgcaaatgagcagtctgaggtctgaggacacagccatgtattactgtgcaagggatcaggactacggtactatttactatgctatggactactggggtcaaggaacctcagtcaccgtctcctca

SEQ ID NO: 95 - protein, 112 aa, Artificial Sequence
  Synthetic
DVVMTQTPLTLSVTIGQPASISCKSSQSLLYSDGKTYLNWLLQSPGQSPKLLIYLVSELESGVPDRFSGSGSGTDFTLKISRVEAEDLGVYYCVQGTHFPWTFGGGTKLEIK

SEQ ID NO: 96 - protein, 16 aa, Artificial Sequence
  Synthetic
KSSQSLLYSDGKTYLN

SEQ ID NO: 97 - protein, 7 aa, Artificial Sequence
  Synthetic
LVSELES

SEQ ID NO: 98 - protein, 9 aa, Artificial Sequence
  Synthetic
VQGTHFPWT

SEQ ID NO: 99 - DNA, 336 bp, Artificial Sequence
  Synthetic
gatgttgtgatgacccagactccactcactttgtctgttaccattggacagccagcttccatttcttgcaagtcaagtcagagcctcttatatagtgatggaaaaacctatttgaattggttattacagagtccaggccagtctccaaagctcctaatctatctggtgtctgaactggaatctggagtccctgacagattcagtggcagtggatcagggacagattttacactgaaaatcagcagagtggaggctgaggatttgggagtttattactgcgtgcaaggtacacatttcccgtggacgttcggtggaggcaccaagctggaaatcaaa

SEQ ID NO: 100 - protein, 119 aa, Artificial Sequence
  Synthetic
EVQLQQSGTVLARPGASVKMSCKASGYTFTNYWMHWVKQRPGQGLEWIGAIYPGNSDTTYNQKFKGKAKLTAVTSATTAYMELSSLTNEGSAVYYCTREGDYVYAMDYWGQGTSVTVSS

SEQ ID NO: 101 - protein, 5 aa, Artificial Sequence
  Synthetic
NYWMH

SEQ ID NO: 102 - protein, 17 aa, Artificial Sequence
  Synthetic
AIYPGNSDTTYNQKFKG

SEQ ID NO: 103 - protein, 10 aa, Artificial Sequence
  Synthetic
EGDYVYAMDY

SEQ ID NO: 104 - DNA, 357 bp, Artificial Sequence
  Synthetic
gaggttcagctccagcagtctgggactgtgctggcaaggcctggggcttcagtgaagatgtcctgcaaggcttctggctacacctttaccaactactggatgcactgggtaaaacagaggcctggacagggtctggaatggattggcgctatttatcctggaaatagtgatactacctacaaccagaagttcaagggcaaggccaaactgactgcagtcacatctgccaccactgcctacatggaactcagcagcctgacaaatgagggctctgcggtctattactgtacaagagagggtgattacgtctatgctatggactactggggtcaaggaacctcagtcaccgtctcctca

SEQ ID NO: 105 - protein, 107 aa, Artificial Sequence
  Synthetic
DIVMTQSQKFMSTSVGDRVSITGKASQNVGTALAWYQQKPGQSPKLLIYSASNRYTGVPDRFTGSGSGTDFTLTISNMQSEDLADYFCQQYSSYPLTIGAGTKLELR

SEQ ID NO: 106 - protein, 11 aa, Artificial Sequence
  Synthetic
KASQNVGTALA

SEQ ID NO: 107 - DNA, 321 bp, Artificial Sequence
  Synthetic
gacattgtgatgacccagtctcaaaaattcatgtccacatcagtaggagacagggtcagcatcaccggcaaggccagtcagaatgtgggtactgctttagcctggtatcaacagaaaccaggacaatctcctaaactactgatttactcggcatccaatcggtacactggagtccctgatcgcttcacaggcagtggatctgggacagatttcactctcaccatcagcaatatgcagtctgaagacctggcagattatttctgtcagcaatatagtagctatcctctcacgatcggtgctgggaccaagctggagctgaga

SEQ ID NO: 108 - protein, 121 aa, Artificial Sequence
  Synthetic
EVQLQQSGPELVKPGASVKMSCKASGYTFTNYVMHWVKQKPGQGLEWIGYIIPYNDGTIYNEKFRGKATLTSDKFSSTAYMELSSLTSEDSAVYYCARGDNDSDGDAMDYWGQGTSVTVSS

SEQ ID NO: 109 - protein, 17 aa, Artificial Sequence
  Synthetic
YIIPYNDGTIYNEKFRG

SEQ ID NO: 110 - protein, 12 aa, Artificial Sequence
  Synthetic
GDNDSDGDAMDY

SEQ ID NO: 111 - DNA, 363 bp, Artificial Sequence
  Synthetic
gaggtccagctgcagcagtctggacctgagctggtaaagcctggggcttcagtgaagatgtcctgcaaggcttctggatacacattcactaactatgttatgcactgggtgaagcagaagcctgggcagggccttgagtggattggatatattattccttacaatgatggtactatttacaatgagaaattcagaggcaaggccacactgacttcagacaaattctccagcacagcctacatggagctcagcagcctgacctctgaggactctgcggtctattactgtgcaagaggggataacgactctgatggggatgctatggactactggggtcaaggaacctcagtcaccgtctcctca

SEQ ID NO: 112 - protein, 107 aa, Artificial Sequence
  Synthetic
DIQMTQSPSSLSASLGERVSLTCRASQDIGGSLNWLQQEPDGTIKRLIYATSSLDSGVPKRFSGSRSGSDYSLTISSLESEDFVYYYCLQYASSPLTFGAGTKLELK

SEQ ID NO: 113 - protein, 11 aa, Artificial Sequence
  Synthetic
RASQDIGGSLN

SEQ ID NO: 114 - protein, 9 aa, Artificial Sequence
  Synthetic
LQYASSPLT

SEQ ID NO: 115 - DNA, 321 bp, Artificial Sequence
  Synthetic
gacatccagatgacccagtctccatcctccttatctgcctctctgggagaaagagtcagtctcacttgtcgggcaagtcaggacattggtggtagcttaaactggcttcagcaggaaccagatggaactattaaacgcctgatctacgccacatccagtttagattctggtgtccccaaaaggttcagtggcagtaggtctgggtcagattattctctcaccatcagcagccttgagtctgaagattttgtatactattactgtctacaatatgctagttctccgctcacgttcggtgctgggaccaagctggagctgaaa

SEQ ID NO: 116 - protein, 122 aa, Artificial Sequence
  Synthetic
DVKLVESGGGLVKLGGSLKLSCAASGFTFSSYYMSWVRQTPEKRLEWVATISNSGRSTYYPDTVKGRFTISRDSAKNTLYLQMSSLNSEDTAVYFCARDRDYGYTYEALDYWGQGTSVTVSS

SEQ ID NO: 117 - protein, 5 aa, Artificial Sequence
  Synthetic
SYYMS

SEQ ID NO: 118 - protein, 17 aa, Artificial Sequence
  Synthetic
TISNSGRSTYYPDTVKG

SEQ ID NO: 119 - protein, 13 aa, Artificial Sequence
  Synthetic
DRDYGYTYEALDY

SEQ ID NO: 120 - DNA, 366 bp, Artificial Sequence
  Synthetic
gacgtgaagctcgtggagtctgggggaggcttagtgaagcttggagggtccctgaaactctcctgtgcagcctctggattcactttcagtagctattacatgtcttgggttcgccagactccggagaagaggctggagtgggtcgcaaccattagtaatagtggtcgtagtacctactatccagacactgtgaagggccgattcaccatctccagagacagtgccaagaacaccctgtatctgcaaatgagcagtctgaattctgaggacacagccgtgtatttctgtgcaagagatcgggactacggttatacctacgaagctttggactactggggtcaaggaacctcagtcaccgtctcctca

SEQ ID NO: 121 - protein, 107 aa, Artificial Sequence
  Synthetic
DIQMTQSPSSLSASVGDRVTITCRASENIYSALAWYQQKPGKAPKFLIHNAKTLAEGVPSRFSGSGSGTDFTLTISSLQPEDFATYYCQYHYGIPFTFGQGTKLEIK

SEQ ID NO: 122 - protein, 107 aa, Artificial Sequence
  Synthetic
DIQMTQSPSSLSASVGDRVTITCRASENIYSALAWYQQKPGKAPKFLIHNVKTLAEGVPSRFSGSGSGTDFTLTISSLQPEDFATYYCQYHYGIPFTFGQGTKLEIK

SEQ ID NO: 123 - protein, 107 aa, Artificial Sequence
  Synthetic
DIQMTQSPSSLSASVGDRVTITCRASENIYSALAWYQQKPGKAPKFLIHQAKTLAEGVPSRFSGSGSGTDFTLTISSLQPEDFATYYCQYHYGIPFTFGQGTKLEIK

SEQ ID NO: 124 - protein, 107 aa, Artificial Sequence
  Synthetic
DIQMTQSPSSLSASVGDRVTITCRASENIYSALAWYQQKPGKAPKFLIHSAKTLAEGVPSRFSGSGSGTDFTLTISSLQPEDFATYYCQYHYGIPFTFGQGTKLEIK

SEQ ID NO: 125 - protein, 107 aa, Artificial Sequence [sequence omitted]

SEQ ID NO: 126 - protein, 214 aa, Artificial Sequence
  Synthetic
DIQMTQSPSSLSASVGDRVTITCRASENIYSALAWYQQKPGKAPKFLIHNAKTLAEGVPSRFSGSGSGTDFTLTISSLQPEDFATYYCQYHYGIPFTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 127 - protein, 214 aa, Artificial Sequence
  Synthetic
DIQMTQSPSSLSASVGDRVTITCRASENIYSALAWYQQKPGKAPKFLIHNVKTLAEGVPSRFSGSGSGTDFTLTISSLQPEDFATYYCQYHYGIPFTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 128 - protein, 214 aa, Artificial Sequence
  Synthetic
DIQMTQSPSSLSASVGDRVTITCRASENIYSALAWYQQKPGKAPKFLIHQAKTLAEGVPSRFSGSGSGTDFTLTISSLQPEDFATYYCQYHYGIPFTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 129 - protein, 214 aa, Artificial Sequence
  Synthetic
DIQMTQSPSSLSASVGDRVTITCRASENIYSALAWYQQKPGKAPKFLIHSAKTLAEGVPSRFSGSGSGTDFTLTISSLQPEDFATYYCQYHYGIPFTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 130 - protein, 330 aa, Artificial Sequence [sequence omitted]

SEQ ID NO: 131 - protein, 329 aa, Artificial Sequence
  Synthetic
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEFEGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 132 - protein, 120 aa, Artificial Sequence
  Synthetic
EVQLVESGGGLVKPGGSLRLSCAASGFTFSRYYMSWVRQAPGKGLEWVATISISGGNTYYPDSVKGRFTISRDNAKNTLYLQMSSLRAEDTAVYYCAREGGYGNLWFAYWGQGTLVTVSS

SEQ ID NO: 133 - protein, 120 aa, Artificial Sequence
  Synthetic
EVQLVESGGGLVKPGGSLRLSCAASGFTFSRYYMSWVRQAPGKGLEWVATISISGGNTYYADSVKGRFTISRDNAKNTLYLQMSSLRAEDTAVYYCAREGGYGNLWFAYWGQGTLVTVSS

SEQ ID NO: 134 - protein, 120 aa, Artificial Sequence
  Synthetic
EVQLVESGGGLVKPGGSLRLSCAASGFTFSRYYMSWVRQAPGKGLEWVATISISGGNVYYPDSVKGRFTISRDNAKNTLYLQMSSLRAEDTAVYYCAREGGYGNLWFAYWGQGTLVTVSS

SEQ ID NO: 135 - protein, 120 aa, Artificial Sequence
  Synthetic
EVQLVESGGGLVKPGGSLRLSCAASGFTFSRYYMSWVRQAPGKGLEWVATISISGGQTYYADSVKGRFTISRDNAKNTLYLQMSSLRAEDTAVYYCAREGGYGNLWFAYWGQGTLVTVSS

SEQ ID NO: 136 - protein, 120 aa, Artificial Sequence
  Synthetic
EVQLVESGGGLVKPGGSLRLSCAASGFTFSRYYMSWVRQAPGKGLEWVATISISGGSTYYADSVKGRFTISRDNAKNTLYLQMSSLRAEDTAVYYCAREGGYGNLWFAYWGQGTLVTVSS

SEQ ID NO: 137 - protein, 450 aa, Artificial Sequence
  Synthetic
EVQLVESGGGLVKPGGSLRLSCAASGFTFSRYYMSWVRQAPGKGLEWVATISISGGNTYYPDSVKGRFTISRDNAKNTLYLQMSSLRAEDTAVYYCAREGGYGNLWFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 138 - protein, 449 aa, Artificial Sequence
  Synthetic
EVQLVESGGGLVKPGGSLRLSCAASGFTFSRYYMSWVRQAPGKGLEWVATISISGGNTYYPDSVKGRFTISRDNAKNTLYLQMSSLRAEDTAVYYCAREGGYGNLWFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEFEGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 139 - protein, 450 aa, Artificial Sequence
  Synthetic
EVQLVESGGGLVKPGGSLRLSCAASGFTFSRYYMSWVRQAPGKGLEWVATISISGGNTYYADSVKGRFTISRDNAKNTLYLQMSSLRAEDTAVYYCAREGGYGNLWFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 140 - protein, 449 aa, Artificial Sequence
  Synthetic
EVQLVESGGGLVKPGGSLRLSCAASGFTFSRYYMSWVRQAPGKGLEWVATISISGGNTYYADSVKGRFTISRDNAKNTLYLQMSSLRAEDTAVYYCAREGGYGNLWFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEFEGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 141 - protein, 450 aa, Artificial Sequence
  Synthetic
EVQLVESGGGLVKPGGSLRLSCAASGFTFSRYYMSWVRQAPGKGLEWVATISISGGNVYYPDSVKGRFTISRDNAKNTLYLQMSSLRAEDTAVYYCAREGGYGNLWFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 142 - protein, 449 aa, Artificial Sequence
  Synthetic
EVQLVESGGGLVKPGGSLRLSCAASGFTFSRYYMSWVRQAPGKGLEWVATISISGGNVYYPDSVKGRFTISRDNAKNTLYLQMSSLRAEDTAVYYCAREGGYGNLWFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEFEGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 143 - protein, 450 aa, Artificial Sequence
  Synthetic
EVQLVESGGGLVKPGGSLRLSCAASGFTFSRYYMSWVRQAPGKGLEWVATISISGGQTYYADSVKGRFTISRDNAKNTLYLQMSSLRAEDTAVYYCAREGGYGNLWFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 144 - protein, 449 aa, Artificial Sequence
  Synthetic
EVQLVESGGGLVKPGGSLRLSCAASGFTFSRYYMSWVRQAPGKGLEWVATISISGGQTYYADSVKGRFTISRDNAKNTLYLQMSSLRAEDTAVYYCAREGGYGNLWFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEFEGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 145 - protein, 450 aa, Artificial Sequence
  Synthetic
EVQLVESGGGLVKPGGSLRLSCAASGFTFSRYYMSWVRQAPGKGLEWVATISISGGSTYYADSVKGRFTISRDNAKNTLYLQMSSLRAEDTAVYYCAREGGYGNLWFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 146 - protein, 449 aa, Artificial Sequence
  Synthetic
EVQLVESGGGLVKPGGSLRLSCAASGFTFSRYYMSWVRQAPGKGLEWVATISISGGSTYYADSVKGRFTISRDNAKNTLYLQMSSLRAEDTAVYYCAREGGYGNLWFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEFEGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 147 - protein, 5 aa, Artificial Sequence
  Synthetic
NYVMH